(19) United States
(12) Patent Application Publication
(10) Pub. No.: US 2025/0250542 A1
(43) Pub. Date: Aug. 7, 2025

(54) CULTURE METHOD

(71) Applicant: Quell Therapeutics Limited, London (GB)

(72) Inventors: Nathalie BELMONTE, London (GB); Nia EMAMI, London (GB); Madhav KISHORE, London (GB); Aleks GUVENEL, London (GB)

(21) Appl. No.: 18/435,482

(22) Filed: Feb. 7, 2024

(30) Foreign Application Priority Data
Feb. 7, 2023 (GB) 2301711.4

(51) Int. Cl. C12N 5/0783; C12N 5/10

(52) U.S. Cl. CPC C12N 5/0637; C12N 5/10; C12N 2501/727; C12N 2501/998

(57) ABSTRACT

An in vitro method for culturing regulatory T-cells (Tregs) comprising contacting the Tregs with an mTOR inhibitor, and a product obtained by and/or obtainable by said method.

### TECHNICAL FIELD

The present invention relates generally to an in vitro method for culturing Tregs comprising pre-treating the Tregs with an mTOR inhibitor prior to activation and then culturing the activated Tregs in the presence of an mTOR inhibitor after activation. The in vitro method may particularly be an in vitro method for expanding Tregs comprising pre-treating the Tregs with an mTOR inhibitor prior to activation and then culturing the activated Tregs in the presence of an mTOR inhibitor after activation. In particular, an mTOR inhibitor may be applied to the Tregs only once during the in vitro method, specifically before the Tregs are activated, and may remain present after Treg activation for at least a portion of the culturing step(s). Thus, it may not be necessary to add any additional mTOR inhibitor after the pre-treatment. The present invention further relates to the Treg cell product obtainable by the in vitro method described herein. The present invention additionally relates to the use of the in vitro method described herein to increase Treg expansion or to reduce impurities in the Treg cell product or to promote a desired phenotype or to increase expression of a heterologous nucleic acid that may have been introduced into the Tregs.

### BACKGROUND

Adoptive cell therapy (ACT), that is the administration of functional immune cells to a subject, has become an established and evolving immunotherapeutic approach for various medical conditions, including notably malignant or infectious diseases. Tumour-infiltrating lymphocytes were initially shown to be effective in treating metastatic melanoma, and subsequently redirected T-cells or NK cells expressing chimeric antigen receptors (CARs) or heterologous T-cell receptors (TCRs) to target different cellular target molecules have been developed and adopted for clinical use. Initial approaches used immune cells with cytotoxic properties, e.g. cytotoxic T-cells or NK cells, to target and kill unwanted or deleterious cells in the body, but more recently regulatory T-cells (Tregs), which are CD4+CD25+FOXP3+, have been developed for ACT. Tregs have immunosuppressive function. They act to control cytopathic immune responses and are essential for the maintenance of immunological tolerance. The suppressive properties of Tregs can be exploited therapeutically, for example to improve and/or prevent immune-mediated organ damage in inflammatory disorders, autoimmune diseases, and in transplantation.

The main source of cells for ACT is from a patient's own blood (drawn directly from a blood vessel or as a product of leukapheresis) or from umbilical cord blood. Substantial numbers of cells are required to have a therapeutic effect. In order to obtain sufficient numbers of cells for ACT it is necessary to expand the desired population of cells ex vivo prior to administration to the subject. This is particularly important and challenging for Treg ACT since Tregs represent a very small proportion of an individual's immune cells (around 3% to 5% of the peripheral blood) and substantial numbers are required to suppress the immune system. However, expansion of Tregs may also result in the expansion of other contaminating cells such as CD4+CD25− T-cells and CD8+ T-cells. Depending on the level of contamination in the starting material, there is a risk that the contaminating cells will expand to levels that are not acceptable for ACT, and in some cases may outgrow the desired Tregs.

Currently autologous cells are primarily used over allogeneic cells for ACT to reduce the risk of adverse immune reactions such as rejection of the transplanted cells and graft-versus-host disease (GvHD). However, it has been reported that patients with autoimmune diseases often have particularly low numbers of Tregs and/or Tregs with a reduced suppressive function (see, Brusko et al. 2005, Diabetes; 54: 1407-141; Lindley et al. 2005, Diabetes; 54: 92-99; Viglietta et al. 2004, J. Exp. Med; 199: 971-979; Kriegel et al. 2004, J. Exp. Med; 199; 1286-1291; Balandina et al. 2005, Blood; 105: 735-741). The present inventors have also seen that immune suppressed patients such as liver transplant patients often have reduced circulating Tregs in the blood and thus it is harder to isolate and expand Tregs with high purity, particularly under GMP settings. Due to the lower frequency of Tregs in these patients, higher levels of non-Treg contaminating cells are present in the starting material that has been sorted for Tregs. This creates additional difficulties in the manufacture of Tregs for ACT.

One approach used to promote the in vitro expansion of Tregs over other contaminating T-cells involves culturing the cells in the presence of the mTOR inhibitor rapamycin. Such protocols generally involve adding rapamycin to the culture medium at the same time that the cells are activated and keeping the rapamycin in the culture medium throughout. Battaglia et al. 2006, J. Immunol; 177: 8338-8347 described how in vitro activation of a bulk population of human CD4+ T cells in the presence of rapamycin allowed the expansion of CD4+CD25+FOXP3+ Tregs whilst suppressing the expansion of CD4+CD25− and CD8+T-effector cells. However, rapamycin greatly reduced the rate of proliferation of the Tregs. Therefore, protocols for the expansion of Tregs in the presence of rapamycin require cells to be cultured over long periods of time in order to obtain sufficient numbers of cells for ACT. This is particularly problematic for commercial processes as the additional time to manufacture the product increases the cost of the ACT therapy and delays the start of manufacturing of the next product for the next patient. Further, longer protocols could have a detrimental effect on Treg quality and survival.

It is therefore desirable to provide new and improved methods for the in vitro expansion of Tregs. In particular, it is desirable to provide methods for the in vitro expansion of Tregs which enable the manufacture of a product with a desired number of cells at a desired level of purity (e.g. having low levels of T-effector cells), with a desired phenotypic profile (e.g. level of FOXP3 expression) in the shortest time possible.

### SUMMARY

The present inventors have developed an in vitro method for culturing or expanding Tregs which involves a first step of pre-treating the Treg starting material with an mTOR inhibitor prior to activation and a further step of culturing the activated Tregs in the presence of an mTOR inhibitor. The method advantageously reduces the proportion of contaminating T-effector cells and increases the proportion of FOXP3+ cells in the product compared to methods that do not include both of these steps. The Treg cell product has further been found to have an improved suppressive effect and cytokine expression profile compared to products prepared by methods that do not include both of these steps.

In addition, the in vitro expansion method developed by the inventors may increase cell expansion compared to methods that do not include both of the above steps. Any increased cell expansion provided by the in vitro method developed by the present inventors enables Treg cell products to be prepared with an increased number of cells and/or be prepared in a shorter period of time whilst maintaining cell number compared to products prepared by methods that do not include both of the above steps. This is particularly surprising given rapamycin has previously been disclosed to reduce the rate of expansion of Tregs.

Compared to methods described in the art which involve adding rapamycin at the time of activation and keeping the rapamycin in the culture medium for the entirety of the culture process, in certain embodiments the present inventors have developed a method whereby pre-treating the cells with an mTOR inhibitor enables the rapamycin to be diluted or removed from the culture medium after activation but before the end of the culture process to increase the rate of expansion and/or to increase total cell expansion whilst maintaining a desired level of purity.

The inventors have further identified an in vitro method for genetically engineering and culturing Tregs which, in addition to the advantages described above, improves the expression of one or more transgene(s) that has/have been introduced into the Tregs. The inventors have further identified that the method of the invention can result in a higher transduction efficiency when one or more transgenes are introduced to a population of Tregs as compared to a method that does not include both of the above steps, but surprisingly despite this increase in the transduction efficiency, the viral copy number (VCN) tends to be lower.

In a first aspect, there is provided an in vitro method for culturing regulatory T-cells (Tregs), the method comprising:
(a) contacting a population of Tregs with an mTOR inhibitor prior to activation;
(b) activating the Tregs within the population; and
(c) culturing the population of Tregs from step (b) in the presence of an mTOR inhibitor.

In a further aspect, there is provided an in vitro method for expanding regulatory T-cells (Tregs), the method comprising:
(a) contacting a population of Tregs with an mTOR inhibitor prior to activation;
(b) activating the Tregs within the population; and
(c) culturing the population of Tregs from step (b) in the presence of an mTOR inhibitor.

In certain embodiments, the method further comprises isolating CD4+CD25+ Tregs prior to step (a).

In certain embodiments, the mTOR inhibitor used in step (a) and/or step (c) may be rapamycin or a rapalog. The mTOR inhibitor used in step (a) may be the same as the mTOR inhibitor used in step (c). The initial concentration of the mTOR inhibitor used in step (a) and/or step (c) may be from about 30 nM to about 500 nM.

In certain embodiments, the method does not include a step of removing the mTOR inhibitor used in step (a) prior to step (b) or step (c). In certain embodiments, the method does not include a step of washing the cells (particularly Tregs) from step (a) prior to step (b) or step (c). Thus, in certain embodiments step (b) is performed in the presence of an mTOR inhibitor. In certain embodiments, at least a portion of the mTOR inhibitor used in step (a) is present during step (b) and step (c) and no additional mTOR inhibitor is added for the purpose of step (c). Thus, alternatively viewed, a single administration of an mTOR inhibitor may be provided during the method of the invention, at a time point prior to the activation of the Tregs (prior to step (b)).

In certain embodiments, the population of Tregs is contacted with the mTOR inhibitor in step (a) for any period of time that results in a product of the in vitro method after step (c) (e.g. after step (d) or (e)) with a decreased % of contaminating cells and/or an increased % of Tregs and/or an increased fold expansion and/or an increased number of cells compared to a corresponding method in which step (a) is not performed.

In certain embodiments, the population of Tregs are contacted with the mTOR inhibitor in step (a) for at least about 15 minutes prior to step (b). For example, the population of Tregs may be contacted with the mTOR inhibitor in step (a) for about 30 minutes to about 90 minutes prior to step (b). For example, the population of Tregs may be contacted with the mTOR inhibitor in step (a) for about 60 minutes prior to step (b).

In certain embodiments, step (b) occurs immediately after step (a).

In certain embodiments, the Tregs within the population are activated in step (b) by contacting them with a TCR/CD3 activator and/or a TCR co-stimulator activator. The TCR/CD3 activator may, for example, be an anti-CD3 antibody or CD3-binding fragment thereof. The TCR co-stimulator activator may, for example, be an anti-CD28 antibody or CD28-binding fragment thereof.

In certain embodiments, the population of Tregs are cultured in step (c) for any period of time that results in a product of the in vitro method with a decreased % of contaminating cells and/or an increased % of Tregs compared to a corresponding method in which step (c) is performed in the absence of an mTOR inhibitor.

In certain embodiments, the population of Tregs are cultured in the presence of the mTOR inhibitor in step (c) for at least about 6 hours. For example, the population of Tregs may be cultured in the presence of the mTOR inhibitor in step (c) for about 12 hours to about 72 hours or from about 12 hours to about 60 hours or from about 36 hours to about 60 hours. For example, the population of Tregs may be cultured in the presence of the mTOR inhibitor in step (c) for about 24 hours. For example, the population of Tregs may be cultured in the presence of the mTOR inhibitor in step (c) for about 48 hours.

In certain embodiments, step (c) occurs concurrently with or immediately after step (b). For example, the TCR/CD3 activator and/or TCR co-stimulator activator that is used to activate the Tregs may not be removed prior to step (c). For example, at least a portion of the TCR/CD3 activator and/or TCR co-stimulator activator may be present throughout step (c).

In certain embodiments, the method further comprises a step of reducing the concentration of the mTOR inhibitor or removing the mTOR inhibitor used in step (c) at the end of step (c) and then culturing the population of Tregs after step (c), particularly expanding the population of Tregs after step (c). For example, the method may further comprise removing the mTOR inhibitor used in step (c) and then culturing (particularly expanding) the population of Tregs in the absence of an mTOR inhibitor after step (c). For example, the method may further comprise diluting the mTOR inhibitor used in step (c) and then culturing (particularly expanding) the population of Tregs in the presence of the reduced concentration of the mTOR inhibitor used in step (c). Particularly, the step of reducing the concentration of or removing the mTOR inhibitor is an active step e.g. the concentration may be reduced or removal of the mTOR inhibitor may be made by addition of volume to the population of Tregs in step (c), or by replacement of at least a portion of the culture media i.e. dilution of the mTOR inhibitor in step (c), or washing the population of Tregs. Particularly, the step of reducing the concentration of or removing the mTOR inhibitor is not a passive step of degradation of the mTOR inhibitor or of usage of the mTOR inhibitor by the cell population. Thus, although as discussed herein degradation or use of the mTOR inhibitor may occur, the step of reducing the concentration of or removing the mTOR inhibitor is particularly not achieved by degradation or use.

In certain embodiments, the concentration of the mTOR inhibitor may be reduced to a concentration equal to or less than about 25 nM. In certain embodiments, the concentration of the mTOR inhibitor may be reduced or the mTOR inhibitor may be removed at least about 6 hours after the start of step (c). For example, the concentration of the mTOR inhibitor may be reduced or the mTOR inhibitor may be removed from about 12 hours to about 72 hours or from about 12 hours to about 60 hours or from about 36 hours to about 60 hours after the start of step (c). For example, the concentration of the mTOR inhibitor may be reduced or the mTOR inhibitor may be removed about 48 hours after the start of step (c).

In certain embodiments, the population of Tregs may be cultured (particularly expanded) for at least about 6 days after the concentration of the mTOR inhibitor is reduced or the mTOR inhibitor is removed. For example, the population of Tregs may be cultured (particularly expanded) for from about 8 days to about 36 days or from about 8 days to about 20 days or from about 10 days to about 14 days after the concentration of the mTOR inhibitor is reduced or the mTOR inhibitor is removed.

In certain embodiments, the method further comprises a step of introducing a heterologous nucleic acid into the Tregs. In certain embodiments, the heterologous nucleic acid is introduced by transducing the Tregs with a viral vector comprising the heterologous nucleic acid. In certain embodiments, the heterologous nucleic acid encodes a chimeric antigen receptor (CAR) and/or a FOXP3 polypeptide and/or a safety switch and/or a polypeptide that increases persistence of the cells.

In certain embodiments, the heterologous nucleic acid is introduced into the Tregs after the start of step (b) or after the start of step (c) e.g., at least about 6 hours after the start of step (b) or after the start of step (c). For example, the heterologous nucleic acid may be introduced into the Tregs from about 12 hours to about 72 hours or from about 12 hours to about 60 hours or from about 36 hours to about 60 hours after the start of step (b) and/or the start of step (c). For example, the heterologous nucleic acid may be introduced into the Tregs about 24 hours after the start of step (b) or after the start of step (c). For example, the heterologous nucleic acid may be introduced into the Tregs about 48 hours after the start of step (b) or after the start of step (c). Alternatively viewed, the heterologous nucleic acid may be introduced into the Tregs (e.g. population of Tregs) during step (b) or during step (c).

In certain embodiments, the concentration of the mTOR inhibitor used in step (c) is reduced prior to or at the same time that the heterologous nucleic acid is introduced into the Tregs. For example, the concentration of the mTOR inhibitor used in step (c) may be reduced prior to or at the same as transducing the Tregs. In certain embodiments, the concentration of the mTOR inhibitor may be reduced to a concentration equal to or less than about 25 nM.

In certain embodiments, the mTOR inhibitor used in step (c) is removed before the heterologous nucleic acid is introduced into the Tregs, for example before the Tregs are transduced. In certain embodiments, the mTOR inhibitor used in step (c) is removed immediately before the heterologous nucleic acid is introduced into the Tregs, for example immediately before the Tregs are transduced.

In certain embodiments, the population of Tregs may be cultured (particularly expanded) for at least about 6 days after the heterologous nucleic acid is introduced into the Tregs. For example, the population of Tregs may be cultured (particularly expanded) for from about 8 days to about 36 days or from about 8 days to about 20 days or from about 10 days to about 14 days after the heterologous nucleic acid is introduced into the Tregs.

In certain embodiments, the population of Tregs may be harvested from about 8 days to about 36 days after the start of step (b). For example, the population of Tregs may be harvested from about 8 days to about 22 days or from about 10 days to about 18 days or from about 12 days to about 16 days after the start of step (b). The population of Tregs may, for example, be cryopreserved after they have been harvested.

In a further aspect, there is provided a product obtained by and/or obtainable by the method of the first aspect.

In a further aspect, there is provided a method for reducing the proportion or amount of one or more contaminating cells (e.g. CD8+ T-cells and/or CD4+CD25− T-cells) in a population of Tregs and/or a method of inhibiting the expansion of CD8+ T-cells in a population of Tregs and/or a method of inhibiting the expansion of CD4+CD25− T-cells in a population of Tregs, the method comprising:
(a) contacting a population of Tregs with an mTOR inhibitor prior to activation;
(b) activating the Tregs within the population; and
(c) culturing the population of Tregs from step (b) in the presence of an mTOR inhibitor.

The method may be in accordance with the first aspect of the invention, including any embodiment thereof. In said methods, the starting population of Tregs (i.e., the population of Tregs used in step (a)) may comprise said contaminating cells, CD8+ T-cells or CD4+CD25− T-cells respectively. As used herein, the term “contaminating cell” refers to any cell that is not a Treg and includes, for example, CD8+ T-cells and CD4+CD25− T-cells. Where the method reduces the proportion or amount of contaminating cells in a population of Tregs, the reduction is in comparison to the starting population of Tregs (i.e., the population of Tregs used in step (a)). Alternatively, the reduction may be in comparison to the product of a corresponding method in which step (a) is not performed and/or in which step (c) is performed in the absence of an mTOR inhibitor.

In a further aspect, there is provided a method for increasing the proportion or amount of Tregs (e.g. increasing the proportion or amount of cells expressing FOXP3 and/or increasing the proportion or amount of cells having a demethylated Treg-specific demethylated region (TSDR)) in a population of Tregs, the method comprising:
(a) contacting a population of Tregs with an mTOR inhibitor prior to activation;
(b) activating the Tregs within the population; and
(c) culturing the population of Tregs from step (b) in the presence of an mTOR inhibitor.

The method may be in accordance with the first aspect of the invention, including any embodiment thereof. In said method, the starting population of Tregs (i.e., the population of Tregs used in step (a)) may comprise one or more contaminating cells such as CD8+ T-cells and/or CD4+CD25− T-cells. The increase is in comparison to the starting population of Tregs (i.e., the population of Tregs used in step (a)). Increasing the proportion or amount of Tregs in a population of Tregs may alternatively be referred to as increasing the purity of a population of Tregs. Alternatively, the increase may be in comparison to the product of a corresponding method in which step (a) is not performed and/or in which step (c) is performed in the absence of an mTOR inhibitor.

In a further aspect, there is provided a method for increasing the suppressive function of a population of Tregs, the method comprising:
(a) contacting a population of Tregs with an mTOR inhibitor prior to activation;
(b) activating the Tregs within the population; and
(c) culturing the population of Tregs from step (b) in the presence of an mTOR inhibitor.

The method may be in accordance with the first aspect of the invention, including any embodiment thereof. The increase in suppressive function is in comparison to the starting population of Tregs (i.e., the population of Tregs used in step (a)). Alternatively, the increase may be in comparison to the product of a corresponding method in which step (a) is not performed and/or in which step (c) is performed in the absence of an mTOR inhibitor.

In a further aspect, there is provided a method for increasing the expression of a heterologous nucleic acid, the method comprising:
(a) contacting a population of Tregs with an mTOR inhibitor prior to activation;
(b) activating the Tregs within the population; and
(c) culturing the population of Tregs from step (b) in the presence of an mTOR inhibitor;
   and further comprising a step of introducing a heterologous nucleic acid into the Tregs. The method may be in accordance with the first aspect of the invention, including any embodiment thereof. The increase in expression of a heterologous nucleic acid may be in comparison to a corresponding method in which step (a) is not performed and/or in which step (c) is performed in the absence of an mTOR inhibitor. Particularly, the method may additionally comprise a step of reducing the concentration of the mTOR inhibitor in contact with the population of Tregs or removing the mTOR inhibitor from the population of Tregs, after step (c) and prior to the step of introducing a heterologous nucleic acid into the Tregs. The population of Tregs may be further cultured (particularly expanded) in the presence of the reduced concentration of an mTOR inhibitor or in the absence of any mTOR inhibitor).

In a further aspect, there is provided a method for culturing (particularly expanding) regulatory T-cells (Tregs), the method comprising:
(a) contacting a population of Tregs with an mTOR inhibitor prior to activation;
(b) activating the Tregs within the population; and
(c) culturing the population of Tregs from step (b) in the presence of an mTOR inhibitor;
(d) reducing the concentration of the mTOR inhibitor in contact with the population of Tregs or removing the mTOR inhibitor from contact with the population of Tregs; and
(e) further culturing (particularly expanding) the population of Tregs in the presence of the reduced concentration of an mTOR inhibitor or in the absence of any mTOR inhibitor.

The method may be in accordance with the first aspect of the invention, including any embodiment thereof.

In a further aspect, there is provided a method for culturing (particularly expanding) regulatory T-cells (Tregs), the method comprising:
(a) contacting a population of Tregs with an mTOR inhibitor prior to activation;
(b) activating the Tregs within the population; and
(c) culturing the population of Tregs from step (b) in the presence of an mTOR inhibitor;
(d) reducing the concentration of the mTOR inhibitor in contact with the population of Tregs or removing the mTOR inhibitor from contact with the population of Tregs;
(e) introducing a heterologous nucleic acid into the Tregs; and
(f) further culturing (particularly expanding) the population of Tregs in the presence of the reduced concentration of an mTOR inhibitor or in the absence of any mTOR inhibitor;
   wherein step (e) occurs before, at the same time, or after step (d); and
   wherein step (e) occurs before step (f).

The method may be in accordance with the first aspect of the invention, including any embodiment thereof.

In a further aspect, there is provided a method for increasing the expansion of Tregs, the method comprising:
(a) contacting a population of Tregs with an mTOR inhibitor prior to activation;
(b) activating the Tregs within the population; and
(c) culturing the population of Tregs from step (b) in the presence of an mTOR inhibitor
(d) reducing the concentration of the mTOR inhibitor in contact with the population of Tregs or removing the mTOR inhibitor from contact with the population of Tregs; and
(e) further culturing (particularly expanding) the population of Tregs in the presence of the reduced concentration of an mTOR inhibitor or in the absence of any mTOR inhibitor.

The method may be in accordance with the first aspect of the invention, including any embodiment thereof. The increase in cell expansion may be in comparison to a corresponding method in which step (a) is not performed and/or in which step (c) is performed in the absence of an mTOR inhibitor.

In a further aspect, there is provided a method for increasing the proportion or amount of CD45RA+ Tregs in a population of Tregs, the method comprising:
(a) contacting a population of Tregs with an mTOR inhibitor prior to activation;
(b) activating the Tregs within the population; and
(c) culturing the population of Tregs from step (b) in the presence of an mTOR inhibitor.

The method may be in accordance with the first aspect of the invention, including any embodiment thereof. In said method, the starting population of Tregs (i.e., the population of Tregs used in step (a)) may comprise at least a portion of cells that are CD45RA−. The increase is in comparison to the product of a corresponding method in which step (a) is not performed and/or in which step (c) is performed in the absence of an mTOR inhibitor.

In a further aspect, there is provided a method for increasing the proportion or amount of Helios+ Tregs in a population of Tregs, the method comprising:
(a) contacting a population of Tregs with an mTOR inhibitor prior to activation;
(b) activating the Tregs within the population; and
(c) culturing the population of Tregs from step (b) in the presence of an mTOR inhibitor.

The method may be in accordance with the first aspect of the invention, including any embodiment thereof. In said method, the starting population of Tregs (i.e., the population of Tregs used in step (a)) may comprise at least a portion of cells that are Helios-. The increase may be in comparison to the starting population of Tregs (i.e., the population of Tregs used in step (a)). Alternatively, the increase may be in comparison to the product of a corresponding method in which step (a) is not performed and/or in which step (c) is performed in the absence of an mTOR inhibitor.

In a further aspect, there is provided a method for increasing the proportion or amount of CD27+ Tregs in a population of Tregs, the method comprising:
(a) contacting a population of Tregs with an mTOR inhibitor prior to activation;
(b) activating the Tregs within the population; and
(c) culturing the population of Tregs from step (b) in the presence of an mTOR inhibitor.

The method may be in accordance with the first aspect of the invention, including any embodiment thereof. In said method, the starting population of Tregs (i.e., the population of Tregs used in step (a)) may comprise at least a portion of cells that are CD27−. The increase may be in comparison to the starting population of Tregs (i.e., the population of Tregs used in step (a)). Alternatively, the increase may be in comparison to the product of a corresponding method in which step (a) is not performed and/or in which step (c) is performed in the absence of an mTOR inhibitor.

In a further aspect, there is provided a method for increasing the transduction efficiency of a heterologous nucleic acid, the method comprising:
(a) contacting a population of Tregs with an mTOR inhibitor prior to activation;
(b) activating the Tregs within the population; and
(c) culturing the population of Tregs from step (b) in the presence of an mTOR inhibitor;
   and further comprising a step of introducing a heterologous nucleic acid into the Tregs. The method may be in accordance with the first aspect of the invention, including any embodiment thereof. The increase in transduction efficiency of a heterologous nucleic acid may be in comparison to a corresponding method in which step (a) is not performed and/or in which step (c) is performed in the absence of an mTOR inhibitor. Particularly, the method may additionally comprise a step of reducing the concentration of the mTOR inhibitor in contact with the population of Tregs or removing the mTOR inhibitor from the population of Tregs, after step (c) and prior to or at the same time as the step of introducing a heterologous nucleic acid into the Tregs. The population of Tregs may be further cultured (particularly expanded) in the presence of the reduced concentration of an mTOR inhibitor or in the absence of any mTOR inhibitor).

In a further aspect, there is provided a method for decreasing the vector copy number (VCN) of a heterologous nucleic acid, the method comprising:
(a) contacting a population of Tregs with an mTOR inhibitor prior to activation;
(b) activating the Tregs within the population; and
(c) culturing the population of Tregs from step (b) in the presence of an mTOR inhibitor;
   and further comprising a step of introducing a heterologous nucleic acid into the Tregs. The method may be in accordance with the first aspect of the invention, including any embodiment thereof. The decrease in VCN of a heterologous nucleic acid may be in comparison to a corresponding method in which step (a) is not performed and/or in which step (c) is performed in the absence of an mTOR inhibitor. Particularly, the method may additionally comprise a step of reducing the concentration of the mTOR inhibitor in contact with the population of Tregs or removing the mTOR inhibitor from the population of Tregs, after step (c) and prior to or at the same time as the step of introducing a heterologous nucleic acid into the Tregs. The population of Tregs may be further cultured (particularly expanded) in the presence of the reduced concentration of an mTOR inhibitor or in the absence of any mTOR inhibitor).

### BRIEF DESCRIPTION OF THE FIGURES

FIG. 1 shows the % of CD8+ cells at day 14 for products expanded from bulk Tregs from healthy donors spiked with 5% CD8+ cells under various conditions.

FIG. 2 shows the % of CD8+ cells at day 14 for products expanded from bulk Tregs from patient material under various conditions.

FIG. 3 shows the % of cells expressing FOXP3 at day 14 for products expanded from bulk Tregs from healthy donors spiked with 5% CD8+ cells under various conditions.

FIG. 4 shows the fold expansion of Treg cells at day 14 for products expanded from bulk Tregs from healthy donors spiked with 5% CD8+ cells under various conditions.

FIG. 5 shows the fold expansion of cells at day 14 for products expanded from bulk Tregs obtained from patient material.

FIGS. 6A and 6B respectively show concentration of IL-17 and IFNγ produced by expansion products obtained from bulk Tregs obtained from patient materials.

FIG. 7 shows the suppression of T-effector cell proliferation by Treg cells obtained from expansion products obtained from patient material under various conditions.

FIG. 8 shows % killing obtained using expansion products obtained from patient material under various conditions.

FIGS. 9A and 9B show total fold expansion of RA+ Tregs and RA− Tregs expanded under no rapa (control) or rapa conditions.

FIGS. 10A and 10B show the phenotype of RA+ Tregs expanded under no rapa (control) or rapa conditions and RA− Tregs expanded under no rapa (control) conditions.

FIG. 11 shows the % of FOXP3+ cells at day 14 for products expanded in a GMP-suitable process.

FIG. 12 shows the % of Helios+ cells at day 14 for products expanded in a GMP-suitable process.

FIG. 13 shows the % of demethylation of FOXP3-TSDR at day 14 for products expanded in a GMP-suitable process.

FIG. 14 shows the % of CD8+ cells at day 14 for products expanded in a GMP-suitable process.

FIG. 15 shows the transduction efficiency at day 14 for products expanded in a GMP-suitable process.

FIG. 16 shows VCN at day 14 for products expanded in a GMP-suitable process.

### DETAILED DESCRIPTION

The present invention relates generally to an in vitro method for culturing (particularly expanding) regulatory T-cells (Tregs) which may increase expansion of the Tregs and/or reduce impurities in the final product. The in vitro method includes pre-treating a population of Tregs with an mTOR inhibitor prior to activation and then culturing the population of activated Tregs in the presence of an mTOR inhibitor. In certain embodiments, the concentration of the mTOR inhibitor which has been used in the step of culturing the population of activated Tregs (step (c)) may be reduced (step (d)) and the population of Tregs may be further cultured (particularly expanded) in the presence of the reduced concentration of the mTOR inhibitor (step (e) or (f)). In certain embodiments, the mTOR inhibitor which has been used in the step of culturing the population of activated Tregs (step (c)) may be removed (step (d)) and the population of Tregs may be further cultured (particularly expanded) in the absence of any mTOR inhibitor (step (e) or step (f)).

As used herein, the term “culturing” and “cell culture” refers to an in vitro method for maintaining at least a portion of the cells within the starting material or population and includes proliferating cells (particularly Tregs), particularly to increase the total number of cells (particularly Tregs) compared to the starting material or starting cell population (cell expansion). It will be appreciated that cells within a step or method of culturing before entering a phase of active proliferation, may undergo a phase of resting or recovery from any previous conditions to which they may have been subjected. “Culture” as used herein encompasses such recovery or rest phases, where cells may not be proliferating and where cell numbers may decrease for a time period (e.g., before proliferation commences) and also encompasses phases where cells are proliferating but the total number of cells has not yet reached the total number of cells in the starting material. At least a portion of cells within the starting material or population refers to the maintenance of at least 50% of cells, particularly of Tregs within the Treg population. For example, at least about 55% or at least about 60% or at least about 65% of cells, particularly of Tregs within the Treg population, may be maintained.

In particular embodiments, where cells are proliferating, the terms “culturing” and “cell culture” may be used interchangeably with “expanding” and “cell expansion” where the total number of cells (e.g. total number of Tregs) has increased compared to the total number of cells (e.g. total number of Tregs) in the starting material. Thus, the in vitro method described herein may alternatively be referred to as an in vitro method for expanding Tregs.

The in vitro method (e.g., culture or expansion) described herein may be carried out in a closed and/or sterile cell culture system. Suitable equipment and conditions for cell culture of Tregs is known to those skilled in the art, particularly equipment used in good manufacturing protocol (GMP) compliant protocols for culturing Tregs, and may be adapted for use with the presently disclosed in vitro method. Suitable tissue culture flasks are also known to those skilled in the art, for example gas-permeable static culture flasks such as the G-rex™. Tregs may be cultured at a temperature ranging from about 30° C. to about 40° C., for example from about 32° C. to about 38° C. or from about 33° C. to about 37° C. or from about 36° C. to about 38° C., for example around 37° C. Tregs may be cultured at about 2% to about 8% CO₂, for example about 3% to about 7% CO₂, for example about 5% CO₂. Tregs may be cultured at about 3% to about 30% 02, for example about 5% to about 25% or about 15% to about 25% 02. Any one or more (e.g. all) of the steps of the method described herein may be performed under any one or more of these conditions, particularly the culture step (c) and any further culture steps. It will be understood that the addition of materials required to carry out the various steps of the method to the population of Tregs (e.g. the addition of an activator in step (b), the addition of the heterologous nucleic acid, the addition of fresh culture media etc.) may take place outside of the desired conditions, however the population of Tregs may be returned to the desired conditions immediately after the required materials have been added to the Tregs.

The in vitro method for culturing or expanding Tregs may provide at least about 2 fold expansion of the starting population of Tregs. For example, the in vitro method for culturing or expanding Tregs may provide at least about 3 fold, 4 fold, 5 fold, 6 fold, 7 fold, 8 fold, 9 fold or 10 fold expansion of the starting population of Tregs. For example, the in vitro method for culturing or expanding Tregs may provide at least about 50 fold, 60 fold, 70 fold, 80 fold, 90 fold or 100 fold expansion of the starting population of Tregs. For example, the in vitro method for culturing or expanding Tregs may provide up to about 3000 fold or up to about 2000 fold or up to about 1000 fold expansion of the starting population of Tregs. Fold expansion of the product (compared to the starting material) can be determined by calculating total number of cells or Tregs at the harvest timepoint and dividing by total number of cells or Tregs respectively seeded at the beginning of step (a).

In particular, there is provided an in vitro method for culturing or expanding regulatory T-cells (Tregs), the method comprising:
(a) contacting a population of Tregs with an mTOR inhibitor prior to activation;
(b) activating the Tregs within the population; and
(c) culturing the population of Tregs from step (b) in the presence of an mTOR inhibitor.

The present invention further relates to an in vitro method for culturing or expanding Tregs wherein the method includes a step of introducing a heterologous nucleic acid into the Tregs (e.g. by transducing with a viral vector). The in vitro method may increase expression of the transgene.

These methods have utility in the preparation of Tregs for adoptive cell transfer.

### Treg Starting Material

“Regulatory T-cells or T-regulatory cells (Tregs)” are immune cells with immunosuppressive function that control cytopathic immune responses and are essential for the maintenance of immunological tolerance. As used herein, the term Treg refers to a T-cell with immunosuppressive function. The term cell with a regulatory phenotype refers to a cell with immunosuppressive function.

Suitably, immunosuppressive function may refer to the ability of the Treg to reduce or inhibit one or more of a number of physiological and cellular effects facilitated by the immune system in response to a stimulus such as a pathogen, an alloantigen, or an autoantigen. Examples of such effects include increased proliferation of conventional T-cells (Tconv) and secretion of proinflammatory cytokines. Any such effects may be used as indicators of the strength of an immune response. A relatively weaker immune response by Tconv in the presence of Tregs would indicate an ability of the Treg to suppress immune responses. For example, a relative decrease in cytokine secretion would be indicative of a weaker immune response, and thus indicative of the ability of Tregs to suppress immune responses. Tregs can also suppress immune responses by modulating the expression of co-stimulatory molecules on antigen presenting cells (APCs), such as B cells, dendritic cells and macrophages. Expression levels of CD80 and CD86 can be used to assess suppression potency of activated Tregs in vitro after co-culture.

Assays are known in the art for measuring indicators of immune response strength, and thereby the suppressive ability of Tregs. In particular, antigen-specific Tconv cells may be co-cultured with Tregs, and a peptide of the corresponding antigen added to the co-culture to stimulate a response from the Tconv cells. The degree of proliferation of the Tconv cells and/or the quantity of a cytokine such as IL-2 they secrete in response to addition of the peptide may be used as indicators of the suppressive abilities of the co-cultured Tregs. Quantity of cytokines may be detected in the cell culture media by ELISA or flow cytometry. An exemplary suppression assay and cytokine analysis is described in the Examples below.

Several different subpopulations of Tregs have been identified which may express different or different levels of particular markers. Tregs generally are T-cells which express the markers CD4, CD25 and FOXP3 (CD4⁺CD25⁺FOXP3⁺).

Tregs may also express CTLA-4 (cytotoxic T-lymphocyte associated molecule-4) or GITR (glucocorticoid-induced TNF receptor).

Treg cells are present in the peripheral blood, lymph nodes, and tissues and Tregs for use herein include thymus-derived, natural Treg (nTreg) cells, peripherally generated Tregs, and induced Treg (iTreg) cells.

A Treg may be identified using the cell surface markers CD4 and CD25 in the absence of or in combination with low-level expression of the surface protein CD127 (CD4⁺CD25⁺CD127⁻ or CD4⁺CD25⁺CD127^{low}). The use of such markers to identify Tregs is known in the art and described in Liu et al. (JEM; 2006; 203; 7(10); 1701-1711), for example.

A Treg may be a CD4⁺CD25⁺FOXP3⁺ T cell, a CD4⁺CD25⁺CD127⁻ T cell, or a CD4⁺CD25⁺FOXP3⁺CD127^{-/low} T cell.

Suitably, the Tregs may be natural Tregs (nTregs). As used herein, the term “natural T reg” means a thymus-derived Treg. Natural T regs are CD4⁺CD25⁺FOXP3⁺ Helios⁺ Neuropilin 1⁺. Compared with iTregs, nTregs have higher expression of PD-1 (programmed cell death-1, pdcd1), neuropilin 1 (Nrp1), Helios (Ikzf2), and CD73. nTregs may be distinguished from iTregs on the basis of the expression of Helios protein or Neuropilin 1 (Nrp1) individually.

The Tregs may have a demethylated Treg-specific demethylated region (TSDR). The TSDR is an important methylation-sensitive element regulating Foxp3 expression (Polansky, J. K., et al., 2008. European journal of immunology, 38(6), pp. 1654-1663).

Further suitable Tregs include, but are not limited to, Tr cells (which do not express Foxp3, and have high IL-10 production); CD8⁺FOXP3⁺ T cells; and γδ FOXP3⁺ T cells.

Different subpopulations of Tregs are known to exist, including naïve Tregs (CD45RA⁺FoxP3^{low}), effector/memory Tregs (CD45RA⁻FoxP3^{high}) and cytokine-producing Tregs (CD45RA⁻FoxP3^{low}). “Memory Tregs” are Tregs which express CD45RO and which are considered to be CD45RO⁺. These cells have increased levels of CD45RO as compared to naïve Tregs (e.g. at least 10, 20, 30, 40, 50, 60, 70, 80 or 90% more CD45RO) and which preferably do not express or have low levels of CD45RA (mRNA and/or protein) as compared to naïve Tregs (e.g. at least 80, 90 or 95% less CD45RA as compared to naïve Tregs). “Cytokine-producing Tregs” are Tregs which do not express or have very low levels of CD45RA (mRNA and/or protein) as compared to naïve Tregs (e.g. at least 80, 90 or 95% less CD45RA as compared to naïve Tregs), and which have low levels of FOXP3 as compared to Memory Tregs, e.g. less than 50, 60, 70, 80 or 90% of the FOXP3 as compared to Memory Tregs. Cytokine-producing Tregs may produce interferon gamma and may be less suppressive in vitro as compared to naïve Tregs (e.g. less than 50, 60, 70, 80 or 90% suppressive than naïve Tregs. Reference to expression levels herein may refer to mRNA or protein expression. Particularly, for cell surface markers such as CD45RA, CD25, CD4, CD45RO etc., expression may refer to cell surface expression, i.e. the amount or relative amount of a marker protein that is expressed on the cell surface. Expression levels may be determined by any known method of the art. For example, mRNA expression levels may be determined by Northern blotting/array analysis, and protein expression may be determined by Western blotting, or preferably by FACS using antibody staining for cell surface expression.

Particularly, the Tregs may be naïve Tregs. “A naïve regulatory T-cell, a naïve T-regulatory cell, or a naïve Treg” as used interchangeably herein refers to a Treg cell which expresses CD45RA (particularly which expresses CD45RA on the cell surface). Naïve Tregs are thus described as CD45RA⁺. Naïve Tregs generally represent Tregs which have not been activated through their endogenous TCRs by peptide/MHC, whereas effector/memory Tregs relate to Tregs which have been activated by stimulation through their endogenous TCRs. Typically, a naïve Treg may express at least 10, 20, 30, 40, 50, 60, 70, 80 or 90% more CD45RA than a Treg cell which is not naïve (e.g. a memory Treg cell). Alternatively viewed, a naïve Treg cell may express at least 2, 3, 4, 5, 10, 50 or 100-fold the amount of CD45RA as compared to a non-naïve Treg cell (e.g. a memory Treg cell). The level of expression of CD45RA can be readily determined by methods of the art, e.g. by flow cytometry using commercially available antibodies. Typically, non-naïve Treg cells do not express CD45RA or low levels of CD45RA.

Particularly, naïve Tregs may not express CD45RO, and may be considered to be CD45RO⁻. Thus, naïve Tregs may express at least 10, 20, 30, 40, 50, 60, 70, 80 or 90% less CD45RO as compared to a memory Treg, or alternatively viewed at least 2, 3, 4, 5, 10, 50 or 100 fold less CD45RO than a memory Treg cell.

Although naïve Tregs express CD25 as discussed above, CD25 expression levels may be lower than expression levels in memory Tregs, depending on the origin of the naïve Tregs. For example, for naïve Tregs isolated from peripheral blood, expression levels of CD25 may be at least 10, 20, 30, 40, 50, 60, 70, 80 or 90% lower than memory Tregs. Such naïve Tregs may be considered to express intermediate to low levels of CD25. However, a skilled person will appreciate that naïve Tregs isolated from cord blood may not show this difference.

Typically, a naïve Treg as defined herein may be CD4⁺, CD25⁺, FOXP3⁺, CD127^{low}, CD45RA⁺.

Low expression of CD127 as used herein refers to a lower level of expression of CD127 as compared to a CD4⁺ non-regulatory or Tcon cell from the same subject or donor. Particularly, naïve Tregs may express less than 90, 80, 70, 60, 50, 40, 30, 20 or 10% CD127 as compared to a CD4⁺ non-regulatory or Tcon cell from the same subject or donor. Levels of CD127 can be assessed by methods standard in the art, including by flow cytometry of cells stained with an anti-CD127 antibody.

Typically, naïve Tregs do not express, or express low levels of CCR4, HLA-DR, CXCR3 and/or CCR6. Particularly, naïve Tregs may express lower levels of CCR4, HLA-DR, CXCR3 and CCR6 than memory Tregs, e.g. at least 10, 20, 30, 40, 50, 60, 70, 80 or 90% lower level of expression.

Naïve Tregs may further express additional markers, including CCR7+ and CD31+.

Isolated naïve Tregs may be identified by methods known in the art, including by determining the presence or absence of a panel of any one or more of the markers discussed above, on the cell surface of the isolated cells. For example, CD45RA, CD4, CD25 and CD127 low can be used to determine whether a cell is a naïve Treg. Methods of determining whether isolated cells are naïve Tregs and methods for determining the presence and/or levels of expression of cell markers are well-known in the art and include, for example, flow cytometry, using commercially available antibodies.

The Tregs that are subjected to the steps the in vitro methods described herein are comprised within a cell population (“a population of Tregs” or “a Treg population”). which comprises a plurality of Tregs. The population of Tregs as described herein can also be interchangeably referred to as a population of cells comprising Tregs. It will be appreciated that not all cells within a Treg population may express the Treg markers described above to the same extent. Thus, a population of Tregs may comprise distinct and identifiable sub-populations of Tregs as defined above. Further, the population of Tregs may not be a pure Treg population and may comprise a level of contaminant cells (non-Treg cells), e.g., CD8+ T cells, CD4+T effector cells, NK cells, APCs etc. For utility in the methods and uses of the invention, it may be sufficient that at least about 50% of the cells in the population of cells are identifiable as Tregs, preferably at least about 60, 70, 75, 80, 90 or 95% of the population are identifiable as Tregs, preferably naïve Tregs.

The starting population of Tregs used in the in vitro method described herein (i.e., the population of Tregs used in step (a)) may be a Treg-enriched population of cells. In certain embodiments, the in vitro method described herein further comprises a step of preparing a Treg-enriched population of cells prior to step (a). This enriched population of cells is then used as the source of the population of Tregs for step (a). The population of cells may be enriched for any of the Treg phenotypes described above.

An “enriched” population of cells refers to a population of cells in which the proportion of certain target cell(s) (e.g. Tregs) in the population is higher (e.g. at least 10, 20, 30, 40 or 50% higher) than in the material it was obtained from. The Treg-enriched population of cells may be prepared by any method known to those of skill in the art and may involve enrichment and/or depletion steps. For example, the Treg-enriched population of cells may be obtained by FACS and/or magnetic bead sorting. A Treg-enriched sample may be generated from the cell-containing sample by any method known to those of skill in the art, for example, from Tcon cells by introducing DNA or RNA coding for FOXP3 and/or from ex vivo differentiation of inducible progenitor cells or embryonic progenitor cells.

The starting population of Tregs (used in step (a)) may, for example, comprise at least about 40% CD4+CD25+ Tregs, for example at least about 50% or at least about 55% or at least about 60% or at least about 65% or at least about 70% or at least about 75% or at least about 80% CD4+CD25+ Tregs. For example, the starting population of Tregs may comprise equal to or less than about 95% or equal to or less than about 90% or equal to or less than about 85% or equal to or less than about 80% CD4+CD25+ Tregs. For example, the starting population of Tregs may comprise from about 50% to about 95% or from about 55% to about 90% CD4+CD25+ Tregs.

The starting population of Tregs may, for example, comprise at least about 25% FOXP3+ T-cells, for example at least about 30% or at least about 40% or at least about 50% or at least about 60% or at least about 70% or at least about 75% or at least about 80% FOXP3+ T-cells. For example, the starting population of Tregs may comprise equal to or less than about 95% or equal to or less than about 90% or equal to or less than about 85% or equal to or less than about 80% FOXP3+ T-cells. For example, the starting population of Tregs may comprise from about 50% to about 95% or from about 55% to about 90% FOXP3+ T-cells.

The Tregs in the starting population of Tregs may particularly express FOXP3. For example, the starting population of Tregs may comprise at least about 25% FOXP3+ Tregs. For example, the starting population of Tregs may comprise at least about 30% or at least about 35% or at least about 40% or at least about 45% or at least about 50% or at least about 55% or at least about 60% or at least about 70% or at least about 75% or at least about 80% FOXP3+ Tregs. For example, the starting population of Tregs may comprise equal to or less than about 95% or equal to or less than about 90% or equal to or less than about 85% FOXP3+ Tregs. For example, the starting population of Tregs may comprise from about 50% to about 95% or from about 55% to about 90% FOXP3+ Tregs. Tregs may have any Treg phenotype as described herein, for example CD4+CD25+FOXP3+.

The starting population of Tregs may, for example, comprise at least about 15% CD45RA+ Tregs (e.g. CD4+CD25+CD127−/lowCD45RA+ Tregs), for example at least about 20% or at least about 25% or at least about 30% or at least about 35% or at least about 40% CD45RA+ Tregs (e.g. CD4+CD25+CD127−/lowCD45RA+ Tregs). For example, the starting population of Tregs may comprise equal to or less than about 95% or equal to or less than about 90% or equal to or less than about 85% or equal to or less than about 80% or equal to or less than about 75% or equal to or less than about 70% or equal to or less than about 65% or equal to or less than about 60% CD45RA+ Tregs (e.g. CD4+CD25+CD127−/lowCD45RA+ Tregs). For example, the starting population of Tregs may comprise from about 50% to about 95% or from about 55% to about 90% CD45RA+ Tregs (e.g. CD4+CD25+CD127−/lowCD45RA+ Tregs).

The starting population of Tregs may, for example, comprise equal to or greater than about 1% CD8+ T-cells, for example equal to or greater than about 2% or equal to or greater than about 3% or equal to or greater than about 4% or equal to or greater than about 5% CD8+ T-cells. For example, the starting population of Tregs may comprise equal to or less than about 15%, 10% or 5% CD8+ T-cells. For example, the starting population of Tregs may comprise from about 1% to about 5, 10 or 15% or from about 2% to about 5, 10 or 15% or from about 3% to about 5, 10 or 15% CD8+ T-cells.

The starting population of Tregs may, for example, comprise equal to or greater than about 1% CD4+CD25− T-cells, for example equal to or greater than about 2% or equal to or greater than about 3% or equal to or greater than about 4% or equal to or greater than about 5% CD4+CD25− T-cells. For example, the starting population of Tregs may comprise equal to or less than about 20%, 10%, 5%, 3% or 2% CD4+CD25− T-cells. For example, the starting population of Tregs may comprise from about 1% to about 20% or from about 2% to about 18% or from about 2% to about 15% CD4+CD25− T-cells.

The in vitro methods described herein may be particularly beneficial where the starting material (population of Tregs) has a particularly high level of impurities (e.g. a high proportion of CD8+ T-cells and/or CD4+CD25− T-cells, and/or a low proportion of CD4+CD25+ T-cells and/or FOXP3+ T-cells). Such starting material is often associated with poor product quality (e.g. with high levels of impurities and lower levels of Tregs) after expansion with standard Treg culture conditions (e.g., in the absence of mTOR inhibitor). In contrast to this, the methods of the invention can produce an expanded Treg population having low levels of impurities and high numbers of Tregs, from a starting material having high levels of impurities and low numbers of Tregs. Thus, in certain embodiments, the starting population of Tregs (e.g. in step (a)) may comprise less than about 60% CD4+CD25+ Tregs and/or less than about 60% CD4+CD25+CD127−/low Tregs and/or less than about 75% FOXP3+ T-cells and/or more than about 5% CD8+ T-cells and/or more than about 10% CD4+CD25− T-cells.

The in vitro methods described herein may be particularly useful to selectively expand CD45RA+ Tregs (e.g. CD4+CD25+CD127−/lowCD45RA+ Tregs) over CD45RA− Tregs (e.g. CD4+CD25+CD127−/lowCD45RA− Tregs). The in vitro methods described herein may therefore result in an expanded Treg population having higher levels of CD45RA+ Tregs (e.g. CD4+CD25+CD45RA+ or CD4+CD25+CD127−/lowCD45RA+) Tregs compared to the expanded Treg population resulting from a corresponding method in which step (a) is not performed and/or step (c) is performed in the absence of mTOR inhibitor. The selective expansion of CD45RA+ Tregs by the in vitro methods described herein may further result in an expanded Treg population having higher levels of markers of a stable Treg phenotype. Particularly, the selective expansion of CD45RA+ Tregs by the in vitro methods described herein may result in an expanded Treg population having higher levels of Helios and/or higher levels of CD27 and/or higher levels of TSDR demethylation compared to the starting population of Tregs used in step (a) and/or in comparison to an expanded Treg population resulting from a corresponding method in which step (a) is not performed and/or step (c) is performed in the absence of mTOR inhibitor.

The population of Tregs that is cultured in the in vitro method described may be isolated from peripheral blood mononuclear cells (PBMCs) obtained from a subject by leukapheresis. The subject from whom the PBMCs are obtained may be a mammal, preferably a human. The Tregs may be matched (e.g. HLA matched) or may be autologous to the subject to whom the cultured Tregs are to be administered. The subject to whom the cultured Tregs are to be administered may be a mammal, particularly a human. The Tregs may be obtained from a patient's own peripheral blood (1^{st} party), or in the setting of a haematopoietic stem cell transplant from donor peripheral blood (2^{nd} party), or peripheral blood from an unconnected donor (3^{rd} party). Suitably, the Tregs are autologous to the subject to whom the Tregs are to be administered. Alternatively, the Tregs cultured or expanded in a method of the invention may be allogeneic, e.g. derived by differentiation of a pluripotent stem cell, particularly from an iPSC.

mTOR Inhibitor

“mTOR” is also known as mammalian target of rapamycin, mechanistic target of rapamycin, FK506-binding protein 12-rapamycin complex-associated protein 1, FKBP 12-rapamycin complex-associated protein, Rapamycin and FKBP12 target 1, Rapamycin target protein 1, FRAP, FRAP1, FRAP2, RAFT1, and RAPT1. In some aspects, the human mTOR protein corresponds to Uniprot No.: P42345. An mTOR inhibitor inhibits, reduces, and/or decreases, and/or is capable of inhibiting, reducing, and/or decreasing at least one activity of mTOR, such as, for example, the serine/threonine protein kinase activity on at least one of its substrates (e.g., p70S6 kinase 1, 4E-BP1, A T/PKB and eEF2). In particular embodiments, an mTOR inhibitor may inhibit, reduce, and/or decrease, and/or is capable of inhibiting, reducing, and/or decreasing an mTOR kinase activity, for example by at least about 10%, 20%, 30%, 40%, 50%, 60%, 70%, 80%, 90%, 95%, 97%, 98% or 99%. This may be determined by a reduction in signal transduction of the mTOR pathway, which is described in more detail below. In some embodiments, the mTOR inhibitor binds directly to and inhibits, and/or is capable of binding directly to and inhibiting mTORC1, mTORC2, or both mTORC1 and mTORC2. Inhibition of mTOR activity by the mTOR inhibitor may be reversible or irreversible.

mTOR is a conserved threonine and serine protein kinase and belongs to the family of phosphatidylinositol-3-kinase-related kinases (PIKKs). mTOR is a protein kinase that phosphorylates threonine and serine residues in its substrates. In certain aspects, mTOR serves as the catalytic subunits of two multi-protein complexes termed as the mTOR complex 1 (mTORC1) and complex 2 (mTORC2). mTORC1 and mTORC2 function independently from each other, despite that fact that, in certain aspect, both mTORC1 and mTORC2 are involved in the phosphoinositol-3 kinase (PI3K) and Akt signaling pathway. In some embodiments, an mTOR inhibitor inhibits, reduces, and/or decreases, and/or is capable of inhibiting, reducing, and/or decreasing an mTORC1 activity, e.g., an mTORC1 kinase activity, and/or an mTORC2 activity.

mTORC1 is a protein complex with five components: mTOR, which is the catalytic subunit of the complex; regulatory-associated protein of mTOR (Raptor); mammalian lethal with Secl3 protein 8 (mLST8, also known as GPL); proline-rich AKT substrate 40 kDa (PRAS40); and DEP-domain-containing mTOR-interacting protein (Deptor). In some embodiments, the mTOR inhibitor prevents the formation of and/or destabilizes the mTORC1 complex.

mTORC2 comprises six different proteins, several of which are common to mTORC1 and mTORC2: mTOR; rapamycin-insensitive companion of mTOR (Rictor); mammalian stress-activated protein kinase interacting protein (mSINI); protein observed with Rictor-1 (Protor-1); mLST8; and Deptor. In particular embodiments, the mTOR inhibitor prevents the formation of and/or destabilizes the mTORC2 complex.

In some embodiments, the mTOR inhibitor is a compound, a small molecule, e.g., small organic molecule, a polynucleotide, an oligonucleotide, an siRNA, a polypeptide, or a fragment, isoform, variant, analog, or derivative thereof that inhibits, reduces, prevents, and/or is capable of inhibiting, reducing, or preventing, one or more activities of mTOR. In some embodiments, the agent is a small molecule. In particular embodiments, the agent is a small molecule with a molecular weight of less than 10 kD, less than 9 kD, less than 8 kD, less than 7 kD, less than 6 kD, less than 5 kD, less than 4 kD, less than 3 kD, less than 2 kD, less than 1 kD, less than 0.5 kD, or less than 0.1 kD. In some embodiments, the agent is a small molecule that is or contains nucleic acids, peptides, polypeptides, peptidomimetics, peptoids, carbohydrates, lipids, components thereof or other organic or inorganic molecules. Libraries of chemical and/or biological mixtures, such as fungal, bacterial, or algal extracts, are known in the art and can be screened for mTOR inhibitor activity. Examples of methods for the synthesis of molecular libraries can be found in: (Carell et al, 1994a; Carell et al, 1994b; Cho et al, 1993; DeWitt et al, 1993; Gallop et al, 1994; Zuckermann et al, 1994).

The mTOR inhibitor may be a nucleic acid, peptide, compound, or small organic molecule that inhibits at least one activity of an mTOR protein, such as, for example, the serine/threonine protein kinase activity on at least one of its substrates (e.g., p70S6 kinase 1, 4E-BP1, A T/PKB and eEF2). mTOR inhibitors may be able to bind directly to and inhibit mTORC1, mTORC2 or both mTORC1 and mTORC2.

Inhibition of mTOR activity, for example mTORC1 and/or mTORC2 activity, can be determined by a reduction in signal transduction of the mTOR pathway. A wide variety of readouts can be utilized to establish a reduction of the output of such signalling pathway. Some non-limiting exemplary readouts include (1) a decrease in phosphorylation of Akt at residues, including but not limited to S473 and T308; (2) a decrease in activation of Akt as evidenced, for example, by a reduction of phosphorylation of Akt substrates including but not limited to Fox01/O3a T24/32, GSK3β; S21/9, and TSC2 T1462; (3) a decrease in phosphorylation of signalling molecules downstream of mTOR (e.g. mTORC1), including but not limited to ribosomal S6 S240/244, 70S6K T389, and 4EBP1 T37/46. Measuring, detecting, and/or assessing proteins with site-specific phosphorylation can be performed by any known means, including, but not limited to, antibody staining techniques and immunoassays, enzyme-linked immunosorbent assay (ELISA), enzyme immunoassay (EIA), radioimmunoassay (RIA), surface plasmon resonance (SPR), Western Blot, or protein array.

In certain embodiments, the mTOR inhibitor has an IC₅₀ of less than 500 pM, less than 200 pM, less than 100 pM, less than 50 μM, less than 10 μM, less than 5 μM, less than 1 μM, less than 500 nM, less than 200 nM, less than 100 nM, less than 50 nM, less than 10 nM, less than 5 nM, less than 1 nM, or less than 500 μM. In certain embodiments, the mTOR inhibitor has an IC₅₀ of between 1 nM and 500 μM, between 1 nM and 500 nM, between 1 μM and 500 μM, between 10 μM and 100 μM, between 100 nM and 1 μM, between 250 nM and 750 nM, between 50 nM and 200 nM, or between 400 nM and between 600 nM. In some embodiments, IC₅₀ determinations can be accomplished using any known standard and/or conventional techniques. For example, in some embodiments, an IC₅₀ can be determined by measuring the mTOR activity in the presence of a range of concentrations of the inhibitor under study. The experimentally obtained values of enzyme activity then are plotted against the inhibitor concentrations used. The concentration of the inhibitor that shows 50% enzyme activity (as compared to the activity in the absence of any inhibitor) is taken as the “IC₅₀” value. Analogously, other inhibitory concentrations can be defined through appropriate determinations of activity. In some embodiments, the IC₅₀ is measured in a cell free assay. In particular embodiments, the IC₅₀ is measured in a cell culture assay. In certain embodiments, the cell culture is a T cell culture, e.g., a primary T cell culture.

Illustrative examples of mTOR inhibitors suitable for use in particular embodiments contemplated herein include, but are not limited to AZD8055, INK128, PF-04691502, and everolimus.

The mTOR inhibitor used in steps (a) and/or (c) of the in vitro method described herein may particularly be rapamycin or a rapalog.

In certain embodiments, the mTOR inhibitor is a selective mTOR inhibitor and may selectively inhibit at least one mTOR activity, e.g. it does not inhibit any additional kinases (e.g. PI3K).

Thus, in some embodiments, the mTOR inhibitor does not inhibit PI3K activity. In certain embodiments, the mTOR inhibitor does not detectably reduce, inhibit, or decrease PI3K activity at the IC₅₀ for mTOR activity. In particular embodiments, the mTOR inhibitor has an IC₅₀ for PI3K activity that is at least 50%, at least 60%, at least 70%, at least 75%, at least 80%, at least 85%, at least 90%, at least 100%, at least 150%, at least 1-fold, at least 2-fold, at least 3-fold, at least 4-fold, at least 5-fold, at least 10-fold, at least 50-fold, or at least 100-fold greater than the IC₅₀ for an mTOR activity. In some embodiments, the mTOR inhibitor inhibits, e.g., selectively inhibits, mTORC1 and mTORC2 kinase activity relative to PI3K activity. In certain embodiments, the inhibitor of mTOR activity is a pyrazolopyrimidine, Torin 1, Torkinib (PP242), PP30, Ku-0063794, WAY-600 (Wyeth), WAY-687 (Wyeth), WAY-354 (Wyeth), or AZD8055.

In particular embodiments, the mTOR inhibitor selectively inhibits mTORC1 with an IC₅₀ of less than 500 pM, less than 200 pM, less than 100 pM, less than 50 μM, less than 10 μM, less than 5 μM, less than 1 μM, less than 500 nM, less than 200 nM, less than 100 nM, less than 50 nM, less than 10 nM, less than 5 nM, less than 1 nM, or less than 500 μM. In certain embodiments, the mTOR inhibitor inhibits the activity of mTORC1 with an IC₅₀ of between 1 nM and 500 μM, between 1 nM and 500 nM, between 1 μM and 500 μM, between 10 μM and 100 μM, between 100 nM and 1 μM, between 250 nM and 750 nM, between 50 nM and 200 nM, or between 400 nM and between 600 nM. In some embodiments, the IC₅₀ is measured in a cell free assay. In particular embodiments, the IC₅₀ is measured in a cell culture assay. In certain embodiments, the cell culture is a T cell culture, e.g., a primary T cell culture.

In some embodiments, the mTOR inhibitor selectively inhibits mTORC1 activity relative to mTORC2 and/or PI3K activity. In certain embodiments, the mTOR inhibitor has an IC₅₀ for PI3K activity that is at least 50%, at least 60%, at least 70%, at least 75%, at least 80%, at least 85%, at least 90%, at least 100%, at least 150%, at least 1-fold, at least 2-fold, at least 3-fold, at least 4-fold, at least 5-fold, at least 10-fold, at least 50-fold, or at least 100-fold greater than the IC₅₀ for an mTORC1 activity. In some embodiments, the mTOR inhibitor is rapamycin (sirolimus). In particular embodiments, the mTOR inhibitor is a rapalog.

In one embodiment, the mTOR inhibitors are active site inhibitors. These are mTOR inhibitors that bind to the ATP binding site (also referred to as ATP binding pocket) of mTOR and inhibit the catalytic activity of both mTORC1 and mTORC2. One class of active site inhibitors are dual specificity inhibitors that target and directly inhibit both PI3K and mTOR. Dual specificity inhibitors bind to both the ATP binding site of mTOR and PI3K.

Illustrative examples of such inhibitors include, but are not limited to: imidazoquinazolines, wortmannin, LY294002, PI-103 (Cayman Chemical), SF1126 (Semafore), BGT226 (Novartis), XL765 (Exelixis) and NVP-BEZ235 (Novartis).

Another class of mTOR active site inhibitors suitable for use in the methods contemplated herein selectively inhibit mTORC1 and mTORC2 activity relative to one or more type I phophatidylinositol 3-kinases, e.g., PI3 kinase α, β, γ, or δ. These active site inhibitors bind to the active site of mTOR but not PI3K. Illustrative examples of such inhibitors include, but are not limited to: pyrazolopyrimidines, Torinl (Guertin and Sabatini), PP242 (2-(4-Amino-1-isopropyl-IH-pyrazolo [3,4-d]pyrimidin-3-yl)-1H-indol-5-ol), PP30, Ku-0063794, WAY-600 (Wyeth), WAY-687 (Wyeth), WAY-354 (Wyeth), and AZD8055 (Liu et al, Nature Review, 8, 627-644, 2009).

In one embodiment, a selective mTOR inhibitor refers to an agent that exhibits a 50% inhibitory concentration (IC₅₀) with respect to mTORC1 and/or mTORC2, that is at least 10-fold, at least 20-fold, at least 50-fold, at least 100-fold, at least 1000-fold, or more, lower than the inhibitor's IC₅₀ with respect to one, two, three, or more type I PI3-kinases or to all of the type I PI3-kinases.

mTOR has been shown to demonstrate a robust and specific catalytic activity toward the physiological substrate proteins, p70 S6 ribosomal protein kinase I (p70S6KI) and eIF4E binding protein 1 (4EBP1) as measured by phosphor-specific antibodies in Western blotting. In one embodiment, the inhibitor of the mTOR pathway is a s6 kinase inhibitor selected from the group consisting of: BI-D1870, H89, PF-4708671, FMK, and AT7867.

Another class of mTOR inhibitors for use in the present invention are compounds that specifically bind to the mTOR FRB domain (FKBP rapamycin binding domain). This includes rapamycin (also known as sirolimus, Rapamune, Fyarro, ABI-009) and rapalogs.

As used herein the term “rapalogs” refers to compounds that specifically bind to the mTOR FRB domain (FKBP rapamycin binding domain), are structurally related to rapamycin, and retain the mTOR inhibiting properties. The term “rapalog” excludes rapamycin. Rapalogs include esters, ethers, oximes, hydrazones, and hydroxylamines of rapamycin, as well as compounds in which functional groups on the rapamycin core structure have been modified, for example, by reduction or oxidation. Pharmaceutically acceptable salts of such compounds are also considered to be rapamycin derivatives. Illustrative examples of rapalogs suitable for use in the methods contemplated herein include, without limitation, temsirolimus (CC1779), everolimus (RAD001), deforolimus (AP23573), AZD8055 (AstraZeneca), and OSI-027 (OSI).

In some embodiments, the agent is a molecule that is described in PCT Pub. Nos.: WO2008/051493; WO2008/051494; or WO2010/062571; and/or U.S. Pat. Nos. 7,981,893; 8,372,976; 7,968,556; 8,383,634; 8,110,578; or 8,492,381, all of which are incorporated by reference herein.

In certain embodiments, the mTOR inhibitor has or includes the formula set forth in Formula (I):

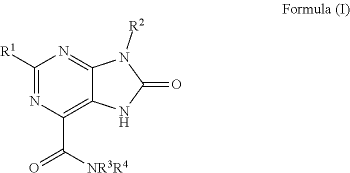

wherein R¹ is substituted or unsubstituted C1-8alkyl, substituted or unsubstituted aryl, substituted or unsubstituted heteroaryl, substituted or unsubstituted cycloalkyl, or substituted or unsubstituted heterocycloalkyl,
R² is substituted or unsubstituted C1-8alkyl, substituted or unsubstituted aryl, substituted or unsubstituted heteroaryl, substituted or unsubstituted cycloalkyl, or substituted or unsubstituted heterocycloalkyl, and
R³ and R⁴ are independently H or C₁₋₈alkyl.

In some embodiments, the mTOR inhibitor is or contains a compound of Formula (I), or a pharmaceutically acceptable salt or solvate thereof. In some embodiments, the mTOR inhibitor is or contains a compound of formula (I) wherein R¹ is substituted aryl, substituted or unsubstituted heteroaryl, such as substituted phenyl. In certain embodiments, the mTOR inhibitor is or contains a compound of formula (I) are those wherein R² is substituted or unsubstituted aryl, such as substituted or unsubstituted phenyl. In particular embodiments, the mTOR inhibitor is or contains a compound of formula (I) wherein groups that are substituted are substituted with one or more halogen; C1-8alkyl; C₂₋₈alkenyl; C₂₋₈alkynyl; hydroxyl; C₁₋₈alkoxyl; amino; nitro; thiol; thioether; imine; cyano; amido; phosphonato; phosphine; carboxyl; thiocarbonyl; sulfonyl; sulfonamide; ketone; aldehyde; ester; carbonyl; haloalkyl; B(OH)₂; carbocyclic cycloalkyl, heterocycloalkyl, monocyclic or fused or non-fused polycyclic aryl or heteroaryl; amino; O-lower alkyl; O-aryl, aryl; aryl-lower alkyl; CO₂CH₃; CONH₂; OCH₂CONH₂; NH₂; SO₂NH₂; OCHF₂; CF₃; or OCF₃groups, wherein each of these groups is optionally substituted.

In some embodiments, the mTOR inhibitor that has or includes the formula set forth in Formula (I) is Compound A. In particular embodiments, the mTOR inhibitor is Compound A. In some aspects, Compound A is 2-(3-Hydroxyphenyl)-9-(2-isopropylphenyl)-8-oxo-8,9-dihydro-7H-purine-6-carboxamide. In some aspects, the mTOR inhibitor is 2-(3-hydroxyphenyl)-9-(2-isopropylphenyl)-8-oxo-8,9-dihydro-7H-purine-6-carboxamide, or a pharmaceutically acceptable salt or solvate thereof. In particular aspects, Compound A has the formula:

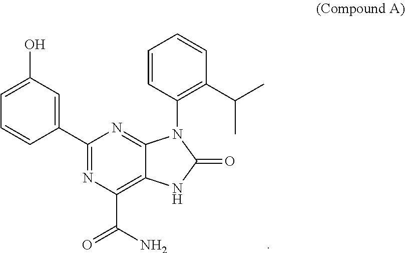 [SMILES: CC(C)c1ccccc1-n1c(=O)[nH]c2c(C(N)=O)nc(-c3cccc(O)c3)nc21]

In particular embodiments, the mTOR inhibitor has or includes the formula set forth in Formula (II):

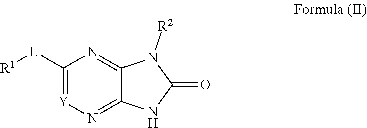

wherein L is a direct bond, NH or O,
Y is N or CR³,
R¹ is H, substituted or unsubstituted C1-8alkyl, substituted or unsubstituted C2-8alkenyl, substituted or unsubstituted aryl, substituted or unsubstituted heteroaryl, substituted or unsubstituted cycloalkyl or substituted or unsubstituted heterocycloalkyl,
R² is H, substituted or unsubstituted C1-8alkyl, substituted or unsubstituted aryl, substituted or unsubstituted heteroaryl, substituted or unsubstituted cycloalkyl, or substituted or unsubstituted heterocycloalkyl,
R³ is H, substituted or unsubstituted C1-8alkyl, substituted or unsubstituted aryl, substituted or unsubstituted heteroaryl, substituted or unsubstituted cycloalkyl, substituted or unsubstituted heterocycloalkyl, —NHR⁴ or —N(R⁴)₂, and
R⁴ is at each occurrence independently substituted or unsubstituted C1-8alkyl, substituted or unsubstituted aryl, substituted or unsubstituted heteroaryl, substituted or unsubstituted cycloalkyl, or substituted or unsubstituted heterocycloalkyl.

In some embodiments, the mTOR inhibitor is or contains a compound of Formula (II), or a pharmaceutically acceptable salt or solvate thereof. In certain embodiments, the mTOR inhibitor has or includes the formula set forth in Formula (II) wherein R¹ is substituted aryl, such as substituted phenyl. In particular embodiments, the mTOR inhibitor has or includes the formula set forth in Formula (II) wherein Y is CH. In some embodiments, the mTOR inhibitor has or incudes the formula set forth in Formula (II) wherein L is a direct bond. In particular embodiments, the mTOR inhibitor has or includes the formula set forth in Formula (II) wherein R¹ is substituted or unsubstituted aryl and R² is C₁₋₈alkyl substituted with one or more substituents selected from alkoxy, amino, hydroxy, cycloalkyl, or heterocycloalkyl.

In certain embodiments, the mTOR inhibitor has or includes the formula set forth in Formula (II) wherein the groups that are “substituted or unsubstituted,” when substituted, they may be substituted with one or more of any substituent. Examples of substituents are those found in the exemplary compounds and embodiments disclosed herein, as well as halo (e.g., chloro, iodo, bromo, or fluoro); C1-8alkyl; C2-8alkenyl; C2-8alkynyl; hydroxyl; C1-8alkoxyl; amino; nitro; thiol; thioether; imine; cyano; amido; phosphonato; phosphine; carboxyl; carbamoyl; carbamate; acetal; urea; thiocarbonyl; sulfonyl; sulfonamide; sulfinyl; ketone; aldehyde; ester; acetyl; acetoxy; oxygen (═O); haloalkyl (e.g., trifluoromethyl); substituted aminoacyl and aminoalkyl; carbocyclic cycloalkyl, which may be monocyclic or fused or non-fused polycyclic (e.g., cyclopropyl, cyclobutyl, cyclopentyl, or cyclohexyl), or a heterocycloalkyl, which may be monocyclic or fused or non-fused polycyclic (e.g., pyrrolidinyl, piperidinyl, piperazinyl, morpholinyl, furanyl, or thiazinyl); carbocyclic or heterocyclic, monocyclic or fused or nonfused polycyclic aryl (e.g., phenyl, naphthyl, pyrrolyl, indolyl, furanyl, thienyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothienyl, or benzofuranyl); amino (primary, secondary, or tertiary); —O-lower alkyl; —O-aryl; aryl; aryl-lower alkyl; CO₂CH₃; CONH₂; OCH₂CONH₂; NH₂; N(C1-4alkyl)₂; NHC(O)C1-4alkyl; SO₂NH₂; SO₂C1-4alkyl; OCHF₂; CF₃; OCF₃; and such moieties may also be optionally substituted by a fused-ring structure or bridge, for example —OCH₂O— or —O-lower alkylene-O—. These substituents may optionally be further substituted with a substituent selected from such groups.

In particular embodiments, the mTOR inhibitor that has or includes the formula set forth in Formula (II) is Compound B. In particular embodiments, the mTOR inhibitor is Compound B. In some aspects, Compound B is 6-(4-(2H-1,2,4-Triazol-3-yl)phenyl)-1-(2-(tetrahydro-2H-pyran-4-yl)ethyl)-1H-imidazo [4,5-b]pyrazine-2(3H)-one. In some aspects, the mTOR inhibitor is 6-(4-(2H-1,2,4-triazol-3-yl)phenyl)-1-(2-(tetrahydro-2H-pyran-4-yl)ethyl)-1H-imidazo [4,5-b]pyrazine-2(3H)-one, or a pharmaceutically acceptable salt or solvate thereof. In particular aspects, Compound B has the formula:

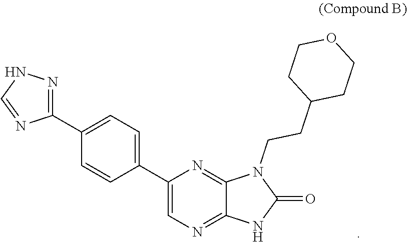 [SMILES: O=c1[nH]c2ncc(-c3ccc(-c4nc[nH]n4)cc3)nc2n1CCC1CCOCC1]

In particular embodiments, the mTOR inhibitor has or includes the formula set forth in Formula (III):

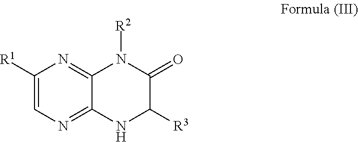

wherein R¹ is substituted or unsubstituted C1-8alkyl, substituted or unsubstituted aryl, substituted or unsubstituted cycloalkyl, substituted or unsubstituted heterocyclyl, or substituted or unsubstituted heterocyclylalkyl,
R² is H, substituted or unsubstituted C1-8alkyl, substituted or unsubstituted cycloalkyl, substituted or unsubstituted heterocyclyl, substituted or unsubstituted heterocyclylalkyl, substituted or unsubstituted aralkyl, or substituted or unsubstituted cycloalkylalkyl, and
R³ is H, or a substituted or unsubstituted C₁₋₈alkyl.

In some embodiments, the mTOR inhibitor is or contains a compound of Formula (III), or a pharmaceutically acceptable salt or solvate thereof. In some embodiments, the mTOR inhibitor has or includes a formula set forth in Formula (III) wherein R¹ is substituted or unsubstituted aryl or substituted or unsubstituted heteroaryl, such as for example, R¹ is phenyl, pyridyl, pyrimidyl, benzimidazolyl, 1H-pyrrolo[2,3-b]pyridyl, indazolyl, indolyl, 1H-imidazo[4,5-b]pyridyl, 1H-imidazo[4,5-b]pyridin-2(3H)-onyl, 3H-imidazo[4,5-b]pyridyl, or pyrazolyl, each optionally substituted. In particular embodiments, the mTOR inhibitor has or includes a formula set forth in Formula (III) wherein R¹ is phenyl substituted with one or more substituents independently selected from the group consisting of substituted or unsubstituted C1-8alkyl (for example, methyl), substituted or unsubstituted heterocyclyl (for example, a substituted or unsubstituted triazolyl or pyrazolyl), aminocarbonyl, halogen (for example, fluorine), cyano, hydroxyalkyl and hydroxy. In other embodiments, R1 is pyridyl substituted with one or more substituents independently selected from the group consisting of substituted or unsubstituted C1-8alkyl (for example, methyl), substituted or unsubstituted heterocyclyl (for example, a substituted or unsubstituted triazolyl), halogen, aminocarbonyl, cyano, hydroxyalkyl (for example, hydroxypropyl), —OR, and —NR2, wherein each R is independently H, or a substituted or unsubstituted C1-4alkyl. In some embodiments, R¹ is 1H-pyrrolo[2,3-b]pyridyl or benzimidazolyl, optionally substituted with one or more substituents independently selected from the group consisting of substituted or unsubstituted C1-8alkyl, and —NR₂, wherein R is independently H, or a substituted or unsubstituted C1-4alkyl.

In some embodiments, the mTOR inhibitor has or includes a formula set forth in Formula (III) wherein R¹ is

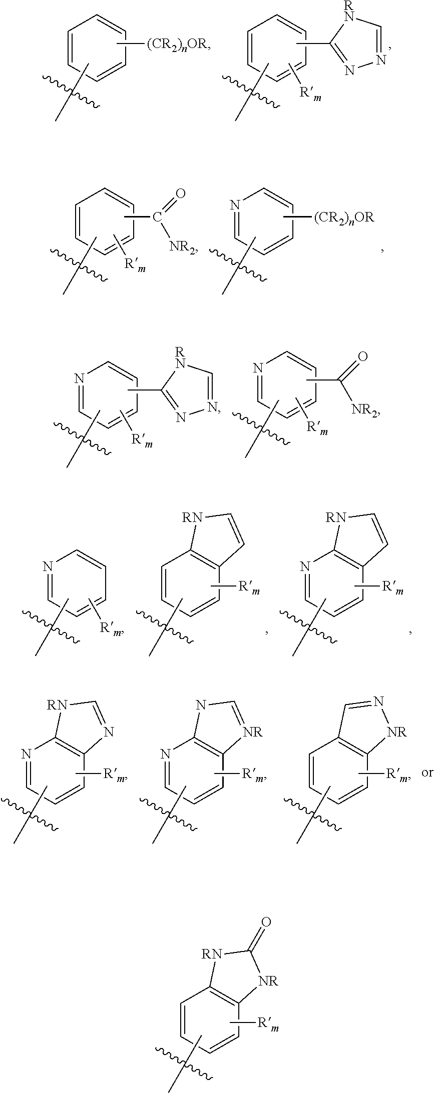

wherein R is at each occurrence independently H, or a substituted or unsubstituted C₁₋₄alkyl (for example, methyl); R¹ is at each occurrence independently a substituted or unsubstituted C1-4 alkyl (for example, methyl), halogen (for example, fluoro), cyano, —OR, or —NR₂; m is 0-3; and n is 0-3. It will be understood that any of the substituents R′ may be attached to any suitable atom of any of the rings in the fused ring systems.

In some embodiments of compounds of formula (III), R² is H, substituted or unsubstituted C1-8alkyl, substituted or unsubstituted cycloalkyl, substituted or unsubstituted heterocyclyl, substituted or unsubstituted C₁₋₄alkyl-heterocyclyl, substituted or unsubstituted C1-4alkyl-aryl, or substituted or unsubstituted C₁₋₄alkyl-cycloalkyl. For example, R² is H, methyl, ethyl, n-propyl, isopropyl, n-butyl, sec-butyl, isobutyl, tert-butyl, n-pentyl, isopentyl, cyclopentyl, cyclohexyl, tetrahydrofuranyl, tetrahydropyranyl, (C₁₋₄alkyl)-phenyl, (C₁₋₄alkyl)-cyclopropyl, (C₁₋₄alkyl)-cyclobutyl, (C₁₋₄alkyl)-cyclopentyl, (C₁₋₄alkyl)-cyclohexyl, (C₁₋₄alkyl)-pyrrolidyl, (C₁₋₄alkyl)-piperidyl, (C₁₋₄alkyl)-piperazinyl, (C₁₋₄alkyl)-morpholinyl, (C₁₋₄alkyl)-tetrahydrofuranyl, or (C₁₋₄alkyl)-tetrahydropyranyl, each optionally substituted.

In certain embodiments, R² is H, C1-4 alkyl, (C1-4alkyl)(OR),

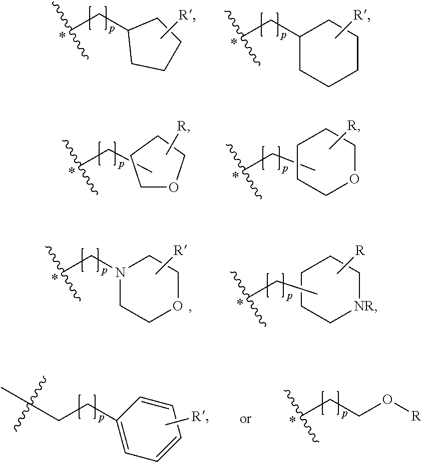

wherein R is at each occurrence independently H, or a substituted or unsubstituted C1-8alkyl, R′ is at each occurrence independently H, —OR, cyano, or a substituted or unsubstituted C1-8alkyl, and p is 0-3.

In particular embodiments, the mTOR inhibitor that has or includes the formula set forth in Formula (III) is Compound C. In particular embodiments, the mTOR inhibitor is Compound C. In some aspects, Compound C is 7-(6-(2-hydroxypropan-2-yl)pyridin-3-yl)-1-((1r,4r)-4-methoxycyclohexyl)-3,4-dihydropyrazino[2,3-b]pyrazin-2(1H)-one. In some aspects, the mTOR inhibitor is 7-(6-(2-hydroxypropan-2-yl)pyridin-3-yl)-1-((1 r,4r)-4-methoxycyclohexyl)-3,4-dihydropyrazino[2,3-b]pyrazin-2(1H)-one, or a pharmaceutically acceptable salt or solvate thereof. In particular aspects, Compound C has the formula:

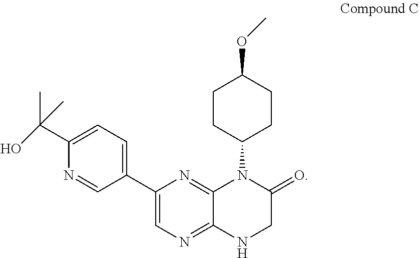 [SMILES: CO[C@H]1CC[C@H](N2C(=O)CNc3ncc(-c4ccc(C(C)(C)O)nc4)nc32)CC1]

In certain embodiments, the mTOR inhibitor used in the in vitro method described herein is rapamycin or a rapalog, particularly rapamycin. In certain embodiments, the mTOR inhibitor used in step (a) is the same as the mTOR inhibitor used in step (c).

In certain embodiments, the in vitro method does not include a step of removing the mTOR inhibitor used in step (a) prior to performing step (b) and/or step (c). For example, the in vitro method may not include a step of washing the Tregs prior to performing step (b) and/or step (c). Thus, in certain embodiments, step (b) is performed in the presence of an mTOR inhibitor. Thus, in certain embodiments, the mTOR inhibitor used in step (a) may also be present during step (b) and/or step (c). Thus in this embodiment, the mTOR inhibitor used in step (c) is the same as the mTOR inhibitor that was used in step (a). In certain embodiments, the mTOR inhibitor used in step (a) remains in culture (is present) during steps (b) and (c) and no additional mTOR inhibitor is required for the purpose of step (c). Alternatively viewed, the method may comprise only a single administration of the mTOR inhibitor (particularly during step (a)). The initial concentration of mTOR inhibitor used in step (a) and/or step (c) may be the same or similar (e.g. the initial concentration of mTOR inhibitor present in step (b) or (c) may be at least 70, 80, 90 or 95% of the initial concentration of mTOR inhibitor present in step (a)). By “initial concentration” it is meant the concentration at the start of the particular step that is referred to. It is noted that the concentration of mTOR inhibitor may naturally decrease with time, for example due to degradation and/or due to uptake of the mTOR inhibitor by cells, and thus the concentration of mTOR inhibitor may be different at the start and end of a particular step. The concentration of the mTOR inhibitor used in a particular step may only decrease naturally during a particular step (e.g. during one or more of steps (a), (b) and/or (c)), in other words the concentration of the mTOR inhibitor may not decrease as a result of any non-natural or external interventions such as dilution (unless stated otherwise as a separate step). The concentration of the mTOR inhibitor may particularly refer to the concentration of the mTOR inhibitor in culture media. In other words, the concentration of the mTOR inhibitor refers to the concentration of the mTOR inhibitor in relation to the total volume in which the cells are contained in. Generally, all concentrations stated herein refer to initial concentrations unless stated otherwise. Concentration of rapamycin in the culture media may be determined by analytical chemistry techniques such as mass spectrometry, for example liquid-chromatography mass spectrometry (LC-MS).

The initial concentration of the mTOR inhibitor used in step (a) and/or step (b) and/or step (c) may, for example, be at least about 30 nM. For example, the initial concentration of the mTOR inhibitor used in step (a) and/or step (b) and/or step (c) may be at least about 40 nM or at least about 50 nM or at least about 60 nM or at least about 70 nM or at least about 80 nM or at least about 90 nM or at least about 100 nM.

The initial concentration of the mTOR inhibitor used in step (a) and/or step (b) and/or step (c) may, for example, be equal to or less than about 500 nM. For example, the initial concentration of the mTOR inhibitor used in step (a) and/or step (b) and/or step (c) may be equal to or less than about 450 nM or equal to or less than about 400 nM or equal to or less than about 350 nM or equal to or less than about 300 nM or equal to or less than about 250 nM or equal to or less than about 200 nM or equal to or less than about 150 nM or equal to or less than about 120 nM.

The initial concentration of the mTOR inhibitor used in step (a) and/or step (b) and/or step (c) may, for example, be from about 30 nM to about 500 nM or from about 50 nM to about 400 nM or from about 75 nM to about 300 nM or from about 75 nM to about 250 nM or from about 75 nM to about 200 nM or from about 75 nM to about 150 nM or from about 80 nM to about 300 nM or from about 80 nM to about 200 nM or from about 80 nM to about 150 nM or from about 80 nM to about 120 nM.

In certain embodiments, mTOR inhibitor is not replenished in the culture media after step (c). In other words, there is no further addition of any mTOR inhibitor in addition to the mTOR inhibitor that is present for the purpose of step (c) (whether the mTOR inhibitor present for step (c) was added at the beginning of step (c) or added as a single administration at step (a)).

### Step (a)

The in vitro method described herein particularly includes the step of contacting a population of Tregs with an mTOR inhibitor prior to activation. The Tregs may, for example, have been obtained from a subject as described above.

“Prior to activation” means that the Tregs (within the population of Tregs) are contacted with an mTOR inhibitor prior to any step of in vitro activation. In other words, the Tregs (within the population of Tregs) are contacted with an mTOR inhibitor before any action has been taken ex vivo to activate the Tregs (e.g. using anti-CD3 and/or anti-CD28 antibodies as described below). This does not include any natural in vivo activation of the Tregs prior to the in vitro method described herein. Thus, some of the Tregs within the population of Tregs that are used in step (a) may have been activated in vivo, before isolation from a subject.

The step of contacting a population of Tregs with an mTOR inhibitor prior to activation is performed in order to pre-treat the cells in the population of Tregs. By “pre-treat” it is meant that the mTOR inhibitor is contacted with the population of Tregs in order to inhibit mTOR in the cells in the population of Tregs. Thus, step (a) may be performed for any period of time and/or with any concentration of mTOR inhibitor that results in inhibition of mTOR in the cells in the population of Tregs.

It may be desirable to inhibit mTOR in as many Tregs as possible, however mTOR may not be inhibited in all of the Tregs within the population and it is not a requirement of the method or this step of the method that mTOR is inhibited in all of the Tregs. Hence, mTOR may be inhibited in a portion of the Tregs present in step (a), e.g. mTOR may be inhibited in at least 50, 60, 70, 80 or 90% of the Tregs.

Inhibition of mTOR may be evaluated by determining the level of phosphorylation of mTOR itself or by determining the level of phosphorylation of downstream targets of mTOR. For example, inhibition of mTOR may be evaluated by determining the level of phosphorylation of p70S6 kinase (p70^{s6k}) and/or the level of phosphorylation of the S6 ribosomal protein (S6). Inhibition of mTOR may be indicated by decreased levels of phosphorylated mTOR (P-mTOR) and/or decreased levels of phosphorylated p70^{s6k} (P-p70^{s6k}) and/or decreased levels of phosphorylated s6 (P-S6) in the pre-treated population of Tregs (i.e. Tregs after step (a) but prior to step (b)) relative to the starting population of Tregs (i.e. the starting population of Tregs used in step (a)).

Levels of phosphorylated protein in a population of Tregs may be visualized on a Western blot by eye and/or may be quantified by densitometric quantification of a Western blot. This may be normalized to a control protein such as tubulin and/or the respective unphosphorylated protein (i.e., P-mTOR would be normalized to unphosphorylated mTOR). Alternatively or additionally, FACS may be used to determine the total number of cells and/or the % of cells in a population that are phosphorylated mTOR-positive and/or phosphorylated p70^{S6K}-positive and/or phosphorylated S6-positive. Suitable antibodies for phosphorylated and unphosphorylated mTOR, p70^{S6K} and S6 are available commercially.

For example, the % of phosphorylated mTOR-positive cells in the population of Tregs following step (a) may decrease by at least about 5 percentage points or at least about 10 percentage points or at least about 15 percentage points or at least about 20 percentage points relative to the starting population of Tregs used in step (a). For example, the % of phosphorylated mTOR-positive cells in the population of Tregs following step (a) may decrease by up to about 80 percentage points or up to about 75 percentage points or up to about 70 percentage points or up to about 65 percentage points or up to about 60 percentage points or up to about 55% percentage points or up to about 50 percentage points relative to the starting population of Tregs used in step (a). For example, the % of phosphorylated mTOR-positive cells in the population of Tregs following step (a) may decrease by 5 to 80 percentage points or by 5 to 70 percentage points or by 10 to 60 percentage points or by 10 to 50 percentage points relative to the starting population of Tregs used in step (a).

For example, the total number of phosphorylated mTOR-positive cells in the population of Tregs following step (a) may decrease by at least about 5% or at least about 10% or at least about 15% or at least about 20% or at least about 25% or at least about 30% or at least about 35% or at least about 40% or at least about 45% or at least about 50% or at least about 55% or at least about 60% relative to the total number of P-mTOR-positive cells in the starting population of Tregs used in step (a). For example, the total number of phosphorylated mTOR-positive cells in the population of Tregs may decrease by up to about 100% or up to about 95% or up to about 90% or up to about 85% or up to about 80% or up to about 75% or up to about 70% or up to about 65% relative to the total number of P-mTOR-positive cells in the starting population used in step (a). For example, the total number of P-mTOR-positive cells in the population of Tregs following step (a) may decrease by 5 to 100% or by 5 to 90% or by 10 to 80% or 10 to 75% or by 15 to 70% relative to the total number of P-mTOR-positive cells in the starting population used in step (a).

For example, the % of phosphorylated mTOR-positive cells and/or the total number of phosphorylated mTOR-positive cells in the population of Tregs following step (a) may decrease to baseline levels of phosphorylation. By baseline levels it is meant the minimum achievable level of phosphorylation of mTOR by the mTOR inhibitor. In other words, increasing the concentration of the mTOR inhibitor and/or increasing the length of time it is in contact with the cells would not decrease the level of phosphorylation of mTOR any further.

For example, the % of phosphorylated p70^{S6K}-positive cells in the population of Tregs following step (a) may decrease by at least about 5 percentage points or at least about 10 percentage points or at least about 15 percentage points or at least about 20 percentage points relative to the starting population of Tregs used in step (a). For example, the % of phosphorylated p70^{S6K}-positive cells in the population of Tregs following step (a) may decrease by up to about 80 percentage points or up to about 75 percentage points or up to about 70 percentage points or up to about 65 percentage points or up to about 60 percentage points or up to about 55% percentage points or up to about 50 percentage points relative to the starting population of Tregs used in step (a). For example, the % of phosphorylated p70^{S6K}-positive cells in the population of Tregs following step (a) may decrease by 5 to 80 percentage points or by 5 to 70 percentage points or by 10 to 60 percentage points or by 10 to 50 percentage points relative to the starting population of Tregs used in step (a).

For example, the total number of phosphorylated p70^{S6K}-positive cells in the population of Tregs following step (a) may decrease by at least about 5% or at least about 10% or at least about 15% or at least about 20% or at least about 25% or at least about 30% or at least about 35% or at least about 40% or at least about 45% or at least about 50% or at least about 55% or at least about 60% relative to the total number of P-p70^{S6K}-positive cells in the starting population of Tregs used in step (a). For example, the total number of phosphorylated p70^{S6K}-positive cells in the population of Tregs may decrease by up to about 100% or up to about 95% or up to about 90% or up to about 85% or up to about 80% or up to about 75% or up to about 70% or up to about 65% relative to the total number of P-p70^{S6K}-positive cells in the starting population used in step (a). For example, the total number of P-p70^{S6K}-positive cells in the population of Tregs following step (a) may decrease by 5 to 100% or by 5 to 90% or by 10 to 80% or 10 to 75% or by 15 to 70% relative to the total number of P-p70^{S6K}-positive cells in the starting population used in step (a).

For example, the % of phosphorylated p70^{S6K}-positive cells and/or the total number of phosphorylated p70^{S6K}-positive cells in the population of Tregs following step (a) may decrease to baseline levels of phosphorylation. By baseline levels it is meant the minimum achievable level of phosphorylation of p70^{S6K} by the mTOR inhibitor. In other words, increasing the concentration of the mTOR inhibitor and/or increasing the length of time it is in contact with the cells would not decrease the level of phosphorylation of p70^{S6K} any further.

For example, the % of phosphorylated S6-positive cells in the population of Tregs following step (a) may decrease by at least about 5 percentage points or at least about 10 percentage points or at least about 15 percentage points or at least about 20 percentage points relative to the starting population of Tregs used in step (a). For example, the % of phosphorylated S6-positive cells in the population of Tregs following step (a) may decrease by up to about 80 percentage points or up to about 75 percentage points or up to about 70 percentage points or up to about 65 percentage points or up to about 60 percentage points or up to about 55% percentage points or up to about 50 percentage points relative to the starting population of Tregs used in step (a). For example, the % of phosphorylated S6-positive cells in the population of Tregs following step (a) may decrease by 5 to 80 percentage points or by 5 to 70 percentage points or by 10 to 60 percentage points or by 10 to 50 percentage points relative to the starting population of Tregs used in step (a).

For example, the total number of phosphorylated S6-positive cells in the population of Tregs following step (a) may decrease by at least about 5% or at least about 10% or at least about 15% or at least about 20% or at least about 25% or at least about 30% or at least about 35% or at least about 40% or at least about 45% or at least about 50% or at least about 55% or at least about 60% relative to the total number of P-S6-positive cells in the starting population of Tregs used in step (a). For example, the total number of phosphorylated S6-positive cells in the population of Tregs may decrease by up to about 100% or up to about 95% or up to about 90% or up to about 85% or up to about 80% or up to about 75% or up to about 70% or up to about 65% relative to the total number of P-S6-positive cells in the starting population used in step (a). For example, the total number of P-S6-positive cells in the population of Tregs following step (a) may decrease by 5 to 100% or by 5 to 90% or by 10 to 80% or 10 to 75% or by 15 to 70% relative to the total number of P-S6-positive cells in the starting population used in step (a).

For example, the % of phosphorylated S6-positive cells and/or the total number of phosphorylated S6-positive cells in the population of Tregs following step (a) may decrease to baseline levels of phosphorylation. By baseline levels it is meant the minimum achievable level of phosphorylation of S6 by the mTOR inhibitor. In other words, increasing the concentration of the mTOR inhibitor and/or increasing the length of time it is in contact with the cells would not decrease the level of phosphorylation of S6 any further.

The ratio of phosphorylated protein to unphosphorylated protein may be determined by densitometric quantification of a Western blot.

The ratio of phosphorylated mTOR to unphosphorylated mTOR (P-mTOR/mTOR) following step (a) may be equal to or less than about 1.0. For example, the ratio of P-mTOR/mTOR may be equal to or less than about 0.9 or equal to or less than about 0.8 or equal to or less than about 0.7 or equal to or less than about 0.6 or equal to or less than about 0.5 or equal to or less than about 0.4 or equal to or less than about 0.3. The ratio P-mTOR/mTOR following step (a) may be at least about 0.01 or at least about 0.05 or at least about 0.1. For example, the ratio of P-mTOR/mTOR following step (a) may be from about 0.01 to about 1.0 or from about 0.05 to about 0.8 or from about 0.1 to about 0.5.

The ratio of P-mTOR/mTOR in the population of Tregs following step (a) may decrease by at least about 0.1 relative to the ratio of P-mTOR/mTOR in the starting population of Tregs used in step (a). For example, the ratio of P-mTOR/mTOR in the population of Tregs following step (a) may decrease by at least about 0.2 or at least about 0.3 or at least about 0.4 relative to the ratio of P-mTOR/mTOR in the starting population of Tregs used in step (a). For example, the ratio of mTOR/mTOR in the population of Tregs following step (a) may decrease by up to about 0.9 or by up to about 0.8 or by up to about 0.7 or by up to about 0.6 or by up to about 0.5.

The ratio of P-mTOR/mTOR in the population of Tregs following step (a) may decrease to baseline levels of phosphorylation. By baseline level it is meant the minimum achievable level of P-mTOR/mTOR by the mTOR inhibitor. In other words, increasing the concentration of the mTOR inhibitor and/or increasing the length of time it is in contact with the cells would not decrease the ratio of P-mTOR/mTOR any further.

The ratio of phosphorylated p70^{S6K} to unphosphorylated p70^{S6K} (P-p70^{S6K}/p70^{S6K}) following step (a) may be equal to or less than about 1.0. For example, the ratio of P-p70^{S6K}/p70^{S6K} may be equal to or less than about 0.9 or equal to or less than about 0.8 or equal to or less than about 0.7 or equal to or less than about 0.6 or equal to or less than about 0.5 or equal to or less than about 0.4 or equal to or less than about 0.3. The ratio P-p70^{S6K}/p70^{S6K} following step (a) may be at least about 0.01 or at least about 0.05 or at least about 0.1. For example, the ratio of P-p70^{S6K}/p70^{S6K} following step (a) may be from about 0.01 to about 1.0 or from about 0.05 to about 0.8 or from about 0.1 to about 0.5.

The ratio of P-p70^{S6K}/p70^{S6K} in the population of Tregs following step (a) may decrease by at least about 0.1 relative to the ratio of P-p70^{S6K}/p70^{S6K} in the starting population of Tregs used in step (a). For example, the ratio of P-p70^{S6K}/p70^{S6K} in the population of Tregs following step (a) may decrease by at least about 0.2 or at least about 0.3 or at least about 0.4 relative to the ratio of P-p70^{S6K}/p70^{S6K} in the starting population of Tregs used in step (a). For example, the ratio of P-p70^{S6K}/p70^{S6K} in the population of Tregs following step (a) may decrease by up to about 0.9 or by up to about 0.8 or by up to about 0.7 or by up to about 0.6 or by up to about 0.5.

The ratio of P-p70^{S6K}/p70^{S6K} in the population of Tregs following step (a) may decrease to baseline levels of phosphorylation. By baseline level it is meant the minimum achievable level of P-p70^{S6K}/p70^{S6K} by the mTOR inhibitor. In other words, increasing the concentration of the mTOR inhibitor and/or increasing the length of time it is in contact with the cells would not decrease the ratio of P-p70^{S6K}/p70^{S6K}any further.

The ratio of phosphorylated S6 to unphosphorylated S6 (P-S6/S6) following step (a) may be equal to or less than about 1.0. For example, the ratio of P-S6/S6 may be equal to or less than about 0.9 or equal to or less than about 0.8 or equal to or less than about 0.7 or equal to or less than about 0.6 or equal to or less than about 0.5 or equal to or less than about 0.4 or equal to or less than about 0.3. The ratio P-S6/S6 following step (a) may be at least about 0.01 or at least about 0.05 or at least about 0.1. For example, the ratio of P-S6/S6 following step (a) may be from about 0.01 to about 1.0 or from about 0.05 to about 0.8 or from about 0.1 to about 0.5.

The ratio of P—S6/S6 in the population of Tregs following step (a) may decrease by at least about 0.1 relative to the ratio of P—S6/S6 in the starting population of Tregs used in step (a). For example, the ratio of P—S6/S6 in the population of Tregs following step (a) may decrease by at least about 0.2 or at least about 0.3 or at least about 0.4 relative to the ratio of P—S6/S6 in the starting population of Tregs used in step (a). For example, the ratio of P—S6/S6 in the population of Tregs following step (a) may decrease by up to about 0.9 or by up to about 0.8 or by up to about 0.7 or by up to about 0.6 or by up to about 0.5.

The ratio of P—S6/S6 in the population of Tregs following step (a) may decrease to baseline levels of phosphorylation. By baseline level it is meant the minimum achievable level of P—S6/S6 by the mTOR inhibitor. In other words, increasing the concentration of the mTOR inhibitor and/or increasing the length of time it is in contact with the cells would not decrease the ratio of P—S6/S6 any further.

Step (a) may be performed in the presence of a culture medium suitable for Treg culture. Thus, the mTOR inhibitor may be present in a culture medium suitable for Treg culture. Exemplary culture media that are known to be suitable for culturing Tregs include XVIVO™ media or TexMACS™ media. The culture media may, for example, be supplemented with serum, for example human serum, particularly human AB serum.

The step of contacting the population of Tregs with an mTOR inhibitor prior to activation (step (a)) may occur immediately before the step of activating the Tregs within the population (step (b)). In other words, there may be no further steps carried out between step (a) and step (b). Particularly, the method may not include a “resting” step between step (a) and step (b) in which the Tregs are left in their container with no further action being taken. The Tregs may be activated (step (b)) as soon as the time period for step (a) has expired.

The Tregs may be contacted with an mTOR inhibitor in step (a) for any time period that results in a product of the in vitro method of the invention (e.g. a product at the end of step (c) or any subsequent step after step (c), for example a product at the end of step (d), (e) or (f)) that has one or more of:
decreased % of contaminating cells;
decreased % of CD8+ T-cells;
decreased % of CD4+CD25− T-cells;
increased % of CD4+CD25+ T-cells;
increased % of cells expressing FOXP3;
increased % of cells having a demethylated TSDR;
increased % of cells expressing Helios;
increased % of cells expressing CD27;
increased fold expansion;
increased number of cells;
increased production of immunosuppressive cytokines (e.g. IL-17 and/or IFNγ);
increased suppressive ability; and
increased transgene expression (when the method further includes a step of introducing a heterologous nucleic acid into the Tregs)
   in comparison to the product of a corresponding method in which step (a) is not performed. The “corresponding method in which step (a) is not performed” is performed using the same starting material and is identical to the method to which it is being compared except that step (a) is not performed. The “corresponding method” may thus start with step (b). Thus, any time period for step (a) that results in an improvement (e.g. as set out in the above bullets) in the product is encompassed in the present invention.

In particular, the Tregs may be contacted with an mTOR inhibitor in step (a) for any time period that results in a product of the method of the invention (e.g. a product at the end of step (c) or any subsequent step) that has one or more of:
decreased % of CD8+ T-cells;
increased % of cells expressing FOXP3;
increased % of cells expressing Helios;
increased % of cells expressing CD27;
increased fold expansion;
increased number of cells;
reduced production of proinflammatory cytokines (e.g. IL-17);
increased suppressive ability; and
increased transgene expression (when the method further includes a step of introducing a heterologous nucleic acid into the Tregs)
   in comparison to the product of a corresponding method in which step (a) is not performed.

In particular, the Tregs may be contacted with an mTOR inhibitor in step (a) for any time period that results in a product of the method of the invention (e.g. a product at the end of step (c) or any subsequent step) that has one or more of:
decreased % of CD8+ T-cells;
increased % of cells expressing FOXP3;
increased % of cells expressing Helios;
increased % of cells expressing CD27;
increased fold expansion; and
increased number of cells;
   in comparison to the product of a corresponding method in which step (a) is not performed.

As discussed above, the “product” refers to the Treg population that is obtained at the end of a method of the invention, particularly, the cell population that is obtained at the end of any one of steps (c), (d), (e), or (f).

The % of CD8+ T-cells, CD4+CD25− T-cells, CD4+CD25+ T-cells, CD4+CD25+CD127−/low T-cells and cells expressing FOXP3 and/or Helios and/or CD27 in the product (or in any Treg population) can be determined using any known methods such as fluorescent-activated cell sorting (FACS).

The % of cells having a demethylated TSDR can be determined using a methylation-specific qPCR assay.

Fold expansion (e.g. of the product) (compared to the starting material) can be determined by calculating total number of cells at the harvest timepoint and dividing by total number of cells seeded at the beginning of step (a). This can be determined for all cells or for Tregs only.

Number of cells in the product can be determined using an automated cell counter to calculate the number of cells/mL in the culture media and then multiplying by the total culture media volume.

Production of pro-inflammatory cytokines (e.g. IL-17 and/or IFNγ) (e.g., by the product) can be determined by harvesting the cell culture media and quantifying the cytokines using ELISA or an equivalent assay such as flow cytometric quantification. An example of a suitable method is provided in the Examples below.

Suppressive ability (e.g. of the product) can be determined using any known suppression assay. For example, as discussed above the ability of the Treg to reduce or inhibit one or more of a number of physiological and cellular effects facilitated by the immune system in response to a stimulus such as a pathogen, an alloantigen, or an autoantigen can indicate their immunosuppressive ability. Examples of such effects include increased proliferation of conventional T-cells (Tconv) and secretion of proinflammatory cytokines. Any such effects may be used as indicators of the strength of an immune response. A relatively weaker immune response by Tconv in the presence of Tregs would indicate an ability of the Treg to suppress immune responses. An example of a suitable method is provided in the Examples below.

Transgene expression can be determined by measuring the effect of one or more transgenes, for example the effect of a safety switch as described in the Examples below. Cells having similar transduction efficiencies (e.g. within 5%, 4%, 3%, 2% or 1% of each other) can be compared. Alternatively, transgene expression can be determined by qPCR.

The Tregs may be contacted with the mTOR inhibitor in step (a) for at least about 15 minutes. For example, the Tregs may be contacted with the mTOR inhibitor in step (a) for at least about 20 minutes or at least about 25 minutes or at least about 30 minutes or at least about 35 minutes or at least about 40 minutes or at least about 45 minutes or at least about 50 minutes or at least about 55 minutes or at least about 60 minutes.

The Tregs may be contacted with the mTOR inhibitor in step (a) for equal to or less than about 12 hours. For example, the Tregs may be contacted with the mTOR inhibitor in step (a) for equal to or less than about 11 hours or equal to or less than about 10 hours or equal to or less than about 9 hours or equal to or less than about 8 hours or equal to or less than about 7 hours equal to or less than about 6 hours or equal to or less than about 5 hours or equal to or less than about 4 hours or equal to or less than about 3 hours.

For example, the Tregs may be contacted with the mTOR inhibitor in step (a) for about 15 minutes to about 12 hours or from about 15 minutes to about 6 hours or from about 15 minutes to about 3 hours or from about 30 minutes to about 3 hours. For example, the Tregs may be contacted with the mTOR inhibitor in step (a) for about 30 minutes to about 90 minutes or from about 45 minutes to about 75 minutes.

Step (a) may take place wholly or partially at ambient temperature (e.g. about 18° (to about 26° C.). Step (a) may take place wholly or partially at ambient atmosphere (e.g. about 78% nitrogen, about 21% oxygen and about 1% other gases). Ambient temperature and atmosphere refer to the temperature and atmosphere of the room in which step (a) is carried out. In certain embodiments, step (a) takes place wholly at ambient temperature and atmosphere. Alternatively put, specialist equipment may not be used to control the temperature and atmosphere that the population of Tregs is subjected to during the whole or part of step (a). In alternative embodiments, the initial addition of the mTOR inhibitor such that it is in contact with the population of Tregs may take place at ambient temperature and atmosphere and the population of Tregs may then be subjected to a particular temperature and atmosphere (e.g. the temperature, CO₂ and/or O₂ conditions described above for culture of Tregs), for example by storing in an incubator that can maintain the desired temperature and atmosphere.

### Step (b)

The in vitro method described herein further comprises the step of activating the Tregs (within the population of Tregs) from step (a). The step of activating the Tregs may occur immediately after the Tregs (within the population of Tregs) have been contacted with an mTOR inhibitor (step (a)). In other words, there may be no further steps carried out between step (a) and step (b). Particularly, the method may not include a “resting” step between step (a) and step (b) in which the Tregs are left in their container with no further action being taken. The Tregs may be activated (step (b)) as soon as the time period for step (a) has expired.

It may be desirable to activate as many Tregs as possible, however not all of the Tregs within the population may be activated and it is not a requirement of the method or this step of the method that all of the Tregs are activated. Hence, a portion of the Tregs present may be activated in step (b), e.g. at least 50, 60, 70, 80 or 90% of the Tregs may be activated. As discussed above, some of the Tregs may have been activated in vivo prior to step (b). Other (non-Treg) cells within the population of Tregs may also be activated, although this is not a requirement of the method.

“Activating” the Tregs refers to initiating signalling pathways which are downstream of TCRs, or alternatively put, initiating signalling pathways which are activated as a result of a TCR binding its ligand. This activation may be mediated by stimulating the cells via their T-cell receptors (TCRs) (which may be endogenous TCRs or heterologous TCRs) and thereby mediating signal transduction via the TCR/CD3 complex. This may take place by contacting the Tregs within the population with a TCR/CD3 activator and/or a TCR co-stimulator activator. In particular, this may take place by contacting the Tregs with a TCR/CD3 activator and a TCR co-stimulator activator. Alternatively, or additionally, activating the Tregs may be mediated by stimulating the cells via heterologous receptors that have been introduced into the cells and can initiate signalling pathways that are downstream of TCRs or signalling pathways that are activated as a result of TCR binding its ligand. The heterologous receptor may particularly be a chimeric antigen receptor (CAR). CARs are described in further detail below and include an intracellular signalling domain which typically may be derived from the CD3 zeta chain. Activation of Tregs may therefore take place by contacting the Tregs within the population with a CAR activator which may be the antigen that the CAR is specific for. Other mechanisms that bypass the TCR may also be used to activate the Tregs. For example, ionophores such as ionomycin and/or phorbol myristate acetate (PMA) may be used to activate Tregs.

Step (b) may be performed in the presence of a culture medium suitable for Treg culture. Thus, the TCR/CD3 activator and/or the TCR co-stimulator activator may be present in a culture medium suitable for Treg culture. Exemplary culture media that are known to be suitable for culturing Tregs include XVIVO™ media or TexMACS™ media. The culture media may, for example, be supplemented with serum, for example human serum, particularly human AB serum. The culture medium used for step (b) may be the same as the culture medium used for step (a). In certain embodiments, the Tregs are not washed between step (a) and step (b) and/or the culture medium used in step (a) is not replaced and/or added to prior to step (b). The culture medium used for step (a) may therefore remain in contact with the Tregs during step (b) and the TCR/CD3 activator and/or the TCR co-stimulator activator may be added to that culture medium.

A “TCR/CD3 activator” may be any agent that binds one or more of the TCR alpha chain, TCR beta chain, CD3 gamma chain, CD3 delta chain, CD3 epsilon chain and CD3 zeta chain of the Tregs to stimulate signal transduction via the TCR/CD3 complex. Examples of TCR/CD3 activators include antigens for which the TCRs are specific, anti-CD3 antibodies and CD3-binding fragments of anti-CD3 antibodies. In particular, the TCR/CD3 activator may be an anti-CD3 antibody or a CD3-binding fragment of an anti-CD3 antibody. The TCR/CD3 activator, for example, the anti-CD3 antibody or CD3-binding fragment of an anti-CD3 antibody, may be immobilised on a surface, for example immobilised on the surface of beads. Such beads are commercially available, for example Dynabeads™ are available from ThermoFisher. Alternatively, the TCR/CD3 activator, for example the anti-CD3 antibody or CD3-binding fragment of an anti-CD3 antibody, may be present in solution.

A “TCR co-stimulator activator” may be any agent that binds one or more TCR co-stimulatory receptors such as CD28 and/or CD2 to stimulate signal transduction via the TCR/CD3 complex. Examples of TCR co-stimulator activators include anti-CD28 antibodies, CD28-binding fragments of anti-CD28 antibodies, anti-CD2 antibodies, and CD2-binding fragments of anti-CD2 antibodies. In particular, the TCR co-stimulator activator may be an anti-CD28 antibody or a CD28-binding fragment of an anti-CD28 antibody. The TCR co-stimulator activator, for example the anti-CD28 antibody or CD28-binding fragment of an anti-CD28 antibody, may be immobilised on a surface, for example, immobilised on the surface of beads. Such beads are commercially available, for example Dynabeads™ are available from ThermoFisher. Alternatively, the TCR co-stimulator, for example the anti-CD28 antibody or CD28-binding fragment of an anti-CD28 antibody, may be present in solution.

In a particular embodiment, the TCR/CD3 activator, for example the anti-CD3 antibody or CD3-binding fragment of an anti-CD3 antibody, and the TCR co-stimulator activator, for example the anti-CD28 antibody or CD28-binding fragment of an anti-CD28 antibody, may be immobilised on the same surface, e.g. on the same bead.

In certain embodiments, the TCR/CD3 activator and/or TCR co-stimulator activator is not removed prior to step (c). In certain embodiments, once the Tregs have been contacted with the TCR/CD3 activator and/or TCR co-stimulator activator, at least a portion of the TCR/CD3 activator and/or TCR co-stimulator activator remain in contact with the Tregs throughout the rest of the culture method (i.e. throughout step (c) and any additional steps) until the Tregs are harvested. In certain embodiments, the remaining part of the TCR/CD3 activator and/or TCR co-stimulator activator may be removed when the Tregs are harvested. Where the TCR/CD3 activator and/or TCR co-stimulator activator are present on the surface of beads, the beads may be separated from the rest of the culture materials, for example the beads may be magnetic and thus may be separated using a magnetic force. Where the TCR/CD3 activator and/or TCR co-stimulator activator are antibodies (which may be present on the surface of beads), the antibodies may be internalised by the cells.

Where time periods herein are defined by reference to “activation”, it is meant the time at which the Tregs are first activated ex vivo, e.g. the time at which the Tregs are first contacted with the TCR/CD3 activator and/or the TCR co-stimulator activator (the start of step (b)).

The initial addition of any materials required to carry out step (b) (e.g. the TCR/CD3 activator and/or the TCR co-stimulator activator) may take place at ambient temperature (e.g. about 18° C. to about 26° C.) and at ambient atmosphere (e.g. about 78% nitrogen, about 21% oxygen and about 1% other gases). The population of Tregs may then be subjected to a particular temperature and atmosphere (e.g. the temperature, CO₂ and/or O₂ conditions described above for culture of Tregs), for example by storing in an incubator that can maintain the desired temperature and atmosphere. Particularly, where step (b) occurs concurrently with step (c), step (b) may take place at the desired temperature and atmosphere for step (c) after the initial addition of any materials required to carry out step (b) to the population of Tregs.

### Step (c)

The in vitro method described herein includes the step of culturing the Tregs from step (b) (the in vitro activated Tregs) in the presence of an mTOR inhibitor.

As used herein, the term “culturing” and “cell culture” refers to an in vitro method for maintaining at least a portion of cells and includes proliferating cells (particularly Tregs), particularly to increase the total number of cells (particularly Tregs) compared to the starting material (cell expansion).

In particular embodiments, the terms “culturing” and “cell culture” may be used interchangeably with “expanding” and “cell expansion”.

Step (c) may be performed in the presence of a culture medium suitable for Treg culture. Thus, the mTOR inhibitor may be present in a culture medium suitable for Treg culture. Exemplary culture media that are known to be suitable for culturing Tregs include XVIVO™ media or TexMACS™ media. The culture media may, for example, be supplemented with serum, for example human serum, particularly human AB serum. The culture medium used for step (c) may be the same as the culture medium used for step (a) and/or step (b). In certain embodiments, the Tregs are not washed between step (b) and step (c) and/or the culture medium used in step (b) is not replaced and/or added to prior to step (c). The culture medium used for step (b) may therefore remain in contact with the Tregs during step (c) and the mTOR inhibitor may be added to that culture medium or may remain in that culture medium from step (a). In certain embodiments, the Tregs are not washed between step (a) and step (c) and/or the culture medium used in step (a) is not replaced and/or added to prior to step (c). The culture medium used for step (a) may therefore remain in contact with the Tregs throughout step (b) and step (c) (and optionally throughout any further steps described herein). Thus, the mTOR inhibitor (e.g. present in the culture medium) used for step (a) may also be used in (e.g. present in the culture medium) used in step (c). In certain embodiments, no additional mTOR inhibitor is added to the culture medium used in step (c). Alternatively viewed, in certain embodiments only a single administration of the mTOR inhibitor may be required for the in vitro methods described herein (e.g. added in step (a)).

The step of culturing the Tregs in the presence of an mTOR inhibitor (step (c)) may occur concurrently with or immediately after the step of activating the Tregs (step (b)). In particular, where the TCR/CD3 activator and/or TCR co-stimulator activator used in step (b) are not removed and are present throughout step (c) (and optionally throughout any further steps described herein), the step of culturing the Tregs in the presence of an mTOR inhibitor (step (c)) may occur concurrently with the step of activating the Tregs (step (b)). In other words, steps (b) and (c) may start at the same time. Where, the step of culturing the Tregs in the presence of an mTOR inhibitor (step (c)) occurs immediately after the step of activating the Tregs (step (b)), step (c) may occur immediately after the TCR/CD3 activator and/or TCR co-stimulator activator used in step (b) is removed and there are no further steps of the method between step (b) and step (c).

The Tregs may be cultured in the presence of an mTOR inhibitor in step (c) for any time period that results in a product of the method of the invention that has one or more of:
decreased % of contaminating cells;
decreased % of CD8+ T-cells;
decreased % of CD4+CD25− T-cells;
increased % of CD4+CD25+ T-cells;
increased % of cells expressing FOXP3;
increased % of cells having a demethylated TSDR;
increased % of cells expressing Helios;
increased % of cells expressing CD27;
increased fold expansion;
increased number of cells;
reduced production of pro-inflammatory cytokines (e.g. IL-17);
increased suppressive ability; and
increased transgene expression (when the method further includes a step of introducing a heterologous nucleic acid into the Tregs)
   in comparison to the product of a corresponding method in which step (c) is performed in the absence of an mTOR inhibitor. The “corresponding method in which step (c) is performed in the absence of an mTOR inhibitor” is performed using the same starting material and is identical to the method to which it is being compared except that step (c) is performed in the absence of an mTOR inhibitor. The “corresponding method” may thus include an alternative step (c) in which the activated Tregs from step (b) are cultured for the same period of time and using the same materials (other than mTOR inhibitor) used in the method to which it is being compared. Thus, any time period for step (c) that results in an improvement in the product is encompassed in the present invention.

In particular, the Tregs may be cultured in the presence of an mTOR inhibitor in step (c) for any time period that results in a product of the method of the invention that has one or more of:
decreased % of CD8+ T-cells;
increased % of cells expressing FOXP3;
increased % of cells expressing Helios;
increased % of cells expressing CD27;
increased fold expansion;
increased number of cells;
reduced production of pro-inflammatory cytokines (e.g. IL-17);
increased suppressive ability; and
increased transgene expression (when the method further includes a step of introducing a heterologous nucleic acid into the Tregs)
   in comparison to the product of a corresponding method in which step (c) is performed in the absence of an mTOR inhibitor.

In particular, the Tregs may be cultured with an mTOR inhibitor in step (c) for any time period that results in a product of the method of the invention that has one or more of:
decreased % of CD8+ T-cells;
increased % of cells expressing FOXP3;
increased % of cells expressing Helios;
increased % of cells expressing CD27;
increased fold expansion; and
increased number of cells;
   in comparison to the product of a corresponding method in which step (c) is performed in the absence of an mTOR inhibitor.

The comparative method that does not include step (c) is a method that is performed on the same starting material and that includes all of the same steps of the method except step (c).

The % of CD8+ T-cells, CD4+CD25− T-cells, CD4+CD25+ T-cells, and cells expressing FOXP3 and/or Helios and/or CD27 in the product, fold expansion, number of cells in the product, production of immunosuppressive cytokines, suppressive ability of the product, and transgene expression may be determined as described above in relation to step (a).

The Tregs may be cultured in the presence of an mTOR inhibitor in step (c) for at least about 6 hours. For example, the Tregs may be cultured in the presence of an mTOR inhibitor in step (c) for at least about 8 hours or at least about 10 hours or at least about 12 hours or at least about 14 hours or at least about 16 hours or at least about 18 hours or at least about 20 hours or at least about 22 hours or at least about 24 hours. In certain embodiments, the Tregs may be cultured in the presence of an mTOR inhibitor in step (c) for at least about 26 hours or at least about 28 hours or at least about 30 hours or at least about 32 hours or at least about 34 hours or at least about 36 hours or at least about 38 hours or at least about 40 hours or at least about 42 hours or at least about 44 hours or at least about 46 hours or at least about 48 hours.

The Tregs may be cultured in the presence of an mTOR inhibitor in step (c) for equal to or less than about 6 days. For example, the Tregs may be cultured in the presence of an mTOR inhibitor in step (c) for equal to or less than about 120 hours or equal to or less than about 96 hours or equal to or less than about 84 hours or equal to or less than about 72 hours or equal to or less than about 60 hours or equal to or less than about 48 hours.

For example, the Tregs may be cultured in the presence of an mTOR inhibitor in step (c) for about 6 hours to about 6 days or from about 12 hours to about 72 hours or from about 12 hours to about 60 hours or from about 24 hours to about 72 hours or from about 24 hours to about 60 hours or from about 36 hours to about 60 hours. For example, the Tregs may be cultured in the presence of an mTOR inhibitor in step (c) for about 24 hours or about 48 hours.

### Further Method Steps

After step (c), the in vitro method described herein may further comprise a step of reducing the concentration of the mTOR inhibitor (e.g. by at least 50, 60, 70, 80 or 90% as compared to the concentration of the mTOR inhibitor at the beginning of step (c) or by at least 10, 20, 30, 40, 50, 60, 70, 80 or 90% as compared to the concentration of the mTOR inhibitor at the end of step (c)) or removing the mTOR inhibitor and then further culturing (and particularly expanding) the Tregs. In particular, the in vitro method described herein may further comprise the step of removing the mTOR inhibitor used in step (c) and then further culturing (and particularly expanding) the Tregs in the absence of an mTOR inhibitor after step (c).

The further culture step (in the presence of a reduced concentration of the mTOR inhibitor compared to the initial concentration used in step (c) or in the absence of an mTOR inhibitor) may be performed in the presence of a culture medium suitable for Treg culture. Exemplary culture media that are known to be suitable for culturing Tregs include XVIVO™ media or TexMACS™ media. The culture media may, for example, be supplemented with serum, for example human serum, particularly human AB serum. The culture medium used for this further culture step may be the same as the culture medium used for step (a) and/or step (b) and/or step (c). In certain embodiments, the Tregs are not washed between step (c) and this further culture step and/or the culture medium used in step (c) is not replaced prior to this additional culture step (e.g. the concentration of the mTOR inhibitor may be reduced by dilution/adding additional culture medium or other reagents to the culture medium). The culture medium used in step (c) may therefore remain in contact with the Tregs during this further culture step. Alternatively, the culture medium used in step (c) may be removed either completely or partially from the Tregs, the Tregs may optionally be washed, and fresh culture medium (which may comprise no mTOR inhibitor) may be used for this further culture step (e.g. fresh culture medium may partially or completely replace the culture medium used in step (c)). Particularly, fresh culture medium may replace at least 10, 20, 30, 40, 50, 60, 70, 80 or 90% of the culture medium present during step (c). Where the culture medium used in step (c) is completely or partially removed prior to this further culture step, no mTOR inhibitor may be added to the fresh culture medium such that this further culture step takes place in the absence of an mTOR inhibitor or in the presence of a reduced concentration of mTOR inhibitor. Alternatively, a reduced concentration of an mTOR inhibitor (e.g. the same or a different mTOR inhibitor used in steps (a) and/or (c)) compared to the initial concentration of mTOR inhibitor used in step (c) may be added to the fresh culture medium.

The dilution or removal of the mTOR inhibitor may occur immediately after step (c). In other words, there are no further steps of the method between step (c) and this dilution or removal of mTOR inhibitor step, including no “resting” step in which the Tregs are left in their container with no further action being taken. The mTOR inhibitor may be diluted or removed as soon as the time period for step (c) has expired.

The additional culture (or expansion) step may occur immediately after the mTOR inhibitor has been diluted or removed. In other words, there are no further steps of the method between the dilution or removal step and this additional culture step, including no “resting” step in which the Tregs are left in their container with no further action being taken. The Tregs may be further cultured or expanded in the presence of a reduced concentration of mTOR inhibitor or in the absence of an mTOR inhibitor as soon as the mTOR inhibitor has been diluted or removed respectively.

Where the concentration of the mTOR inhibitor used in step (c) is reduced, the concentration of the mTOR inhibitor may be reduced compared to the initial concentration of mTOR inhibitor used in step (c). Particularly, the concentration of mTOR inhibitor may be reduced further or in addition to any natural reduction in the initial concentration of mTOR inhibitor used in step (c), for example which may occur due to degradation as discussed above. In other words, the concentration of the mTOR inhibitor used in step (c) is reduced as the result of a non-natural or external intervention. Alternatively put, the step of reducing the concentration of mTOR inhibitor or removing the mTOR inhibitor may be an active step. For example, the concentration may be reduced or the mTOR inhibitor may be removed by addition of volume to the population of Tregs in step (c) (e.g. volume of additional culture media), or by replacement of at least a portion of the culture media, or by washing the population of Tregs. Particularly, the step of reducing the concentration of or removing the mTOR inhibitor is not a passive step of degradation of the mTOR inhibitor or usage of the mTOR inhibitor by the cell population. Thus, although as discussed herein degradation or use of the mTOR inhibitor may occur, the step of reducing the concentration of or removing the mTOR inhibitor is particularly not achieved by degradation or use.

For example, the concentration of the mTOR inhibitor used in step (c) may be reduced to a concentration equal to or less than about 25 nM. In other words, the concentration of the mTOR inhibitor following or as a result of the active step of “reducing the concentration of mTOR inhibitor” as described above, is equal to or less than about 25 nM. For example, the concentration may be reduced to a concentration equal to or less than about 20 nM or equal to or less than about 15 nM or equal to or less than about 10 nM or equal to or less than about 5 nM or equal to or less than about 3 nM or equal to or less than about 2 nM or equal to or less than about 1 nM (the concentration of the mTOR inhibitor following or as a result of the active step of “reducing the concentration of mTOR inhibitor” is equal to or less than about 20 nM or equal to or less than about 15 nM or equal to or less than about 10 nM or equal to or less than about 5 nM or equal to or less than about 3 nM or equal to or less than about 2 nM or equal to or less than about 1 nM). Particularly, where a single administration or treatment of mTOR inhibitor is made in a method of the invention in step (a), the reduction of the concentration of the mTOR inhibitor after step (c) may be as compared to the initial concentration used in step (a), after any natural degradation has been accounted for. Particularly, the reduction in concentration may be a reduction of at least 10, 20, 30, 40, 50, 60, 70, 80 or 90%. It will be appreciated, that during the additional culture or expansion step after reducing the concentration of the mTOR inhibitor, the concentration may be reduced further e.g. by further dilution and/or by degradation of the mTOR. The active step of “reducing the concentration of mTOR inhibitor” may be a result of addition of the materials required to introduce a heterologous nucleic acid into the Tregs as described below.

The concentration of the mTOR inhibitor used in step (c) may be reduced (following or as a result of the active step of “reducing the concentration of mTOR inhibitor” as described above) to a concentration equal to or greater than about 0.01 nM. In other words, the concentration of the mTOR inhibitor following the active step of “reducing the concentration of mTOR inhibitor” is equal to or greater than about 0.01 nM. For example, the concentration may be reduced to a concentration equal to or greater than about 0.05 nM or equal to or greater than about 0.1 nM or equal to or greater than about 0.5 nM (the concentration of the mTOR inhibitor following or as a result of the active step of “reducing the concentration of mTOR inhibitor” is equal to or greater than about 0.05 nM or equal to or greater than about 0.1 nM or equal to or greater than about 0.5 nM). The step of “reducing the concentration of mTOR inhibitor” may be a result of addition of the materials required to introduce a heterologous nucleic acid into the Tregs as described below.

For example, the concentration of the mTOR inhibitor may be reduced (following or as a result of the active step of “reducing the concentration of mTOR inhibitor” as described above) to a concentration of from about 0.01 nM to about 25 nM or from about 0.1 nM to about 20 nM or from about 0.5 nM to about 15 nM or from about 0.5 nM to about 10 nM. In other words, the concentration of the mTOR inhibitor following or as a result of the active step of “reducing the concentration of mTOR inhibitor” is from about 0.01 nM to about 25 nM or from about 0.1 nM to about 20 nM or from about 0.5 nM to about 15 nM or from about 0.5 nM to about 10 nM. The step of “reducing the concentration of mTOR inhibitor” may be a result of addition of the materials required to introduce a heterologous nucleic acid into the Tregs as described below. The resulting concentration of the mTOR inhibitor following the active reduction step may be determined relative to the concentration of the mTOR inhibitor used in step (c)) (or step (a) where a single administration of mTOR inhibitor was used) without accounting for any natural degradation of the mTOR inhibitor or uptake of the mTOR inhibitor by the cells.

The concentration of the mTOR inhibitor used in step (c) may be reduced or diluted (following or as a result of the active step of “reducing the concentration of mTOR inhibitor” as described above) by at least 5 fold (5×). For example, the concentration of the mTOR inhibitor used in step (c) may be reduced or diluted by at least 10 fold or at least 15 fold or at least 20 fold. For example, the concentration of the mTOR inhibitor used in step (c) may be reduced or diluted by up to about 60 fold or up to about 50 fold or up to about 40 fold. For example, the concentration of the mTOR inhibitor used in step (c) may be reduced or diluted by from about 5 fold to about 60 fold or from about 15 fold to about 50 fold or from about 20 fold to about 40 fold. The fold reduction or dilution may be determined relative to the concentration of the mTOR inhibitor used in step (c) (or step (a) where a single administration of mTOR inhibitor was used) without accounting for any natural degradation of the mTOR inhibitor or uptake of the mTOR inhibitor by the cells.

As used herein, “removal” of mTOR inhibitor means that the mTOR inhibitor is removed such that the concentration of any remaining mTOR inhibitor is less than 0.01 nM. Where the mTOR inhibitor is removed, the method may involve a step of washing the population of Tregs, and optionally centrifuging the cells and resuspending the cells, for example in fresh Treg media. Particularly, removal of the mTOR may be a complete removal.

The concentration of the mTOR inhibitor may be reduced or the mTOR inhibitor may be removed at least about 6 hours after the start of step (c). For example, the concentration of the mTOR inhibitor may be reduced or the mTOR inhibitor may be removed at least about 8 hours or at least about 10 hours or at least about 12 hours or at least about 14 hours or at least about 16 hours or at least about 18 hours or at least about 20 hours or at least about 22 hours or at least about 24 hours after the start of step (c). In certain embodiments, the concentration of the mTOR inhibitor may be reduced or the mTOR inhibitor may be removed at least about 26 hours or at least about 28 hours or at least about 30 hours or at least about 32 hours or at least about 34 hours or at least about 36 hours or at least about 38 hours or at least about 40 hours or at least about 42 hours or at least about 44 hours or at least about 46 hours or at least about 48 hours, or at least about 50, 52 or 54 hours after the start of step (c).

The concentration of the mTOR inhibitor may be reduced or the mTOR inhibitor may be removed equal to or less than about 6 days after the start of step (c). For example, the concentration of the mTOR inhibitor may be reduced or the mTOR inhibitor may be removed equal to or less than about 120 hours or equal to or less than about 96 hours or equal to or less than about 84 hours or equal to or less than about 72 hours or equal to or less than about 60 hours or equal to or less than about 48 hours after the start of step (c).

For example, the concentration of the mTOR inhibitor may be reduced or the mTOR inhibitor may be removed about 6 hours to about 6 days or from about 12 hours to about 72 hours or from about 12 hours to about 60 hours or from about 24 hours to about 72 hours or from about 24 hours to about 60 hours or from about 36 hours to about 60 hours after the start of step (c).

This further culture or expansion step (in the presence of a reduced concentration of the mTOR inhibitor or in the absence of an mTOR inhibitor) may take place for any period of time sufficient to provide a desired number of cells and/or a desired fold expansion and/or a desired level of purity (e.g. proportion of Tregs and/or proportion of contaminating cells). The desired number of cells, fold expansion and level of purity (e.g. proportion of Tregs and/or proportion of contaminating cells) may be as described herein in relation to the product of the method.

The duration of this further culture or expansion step may, for example, be dependent on the length of time that step (c) was performed for. Particularly, where cells are cultured in the presence of the mTOR inhibitor in step (c) for a long period of time, it may take longer for the cells to recover (and this further culture step may therefore be longer) compared to if the cells were cultured in the presence of the mTOR inhibitor in step (c) for a shorter period of time.

This further culture or expansion step (in the presence of a reduced concentration of the mTOR inhibitor or in the absence of an mTOR inhibitor) may take place for at least about 6 days after the concentration of the mTOR inhibitor is reduced or the mTOR inhibitor is removed. For example, this further culture or expansion step may take place for at least about 7 days or at least about 8 days or at least about 9 days or at least about 10 days or at least about 11 days or at least about 12 days after the concentration of the mTOR inhibitor is reduced or the mTOR inhibitor is removed.

The further culture or expansion step (in the presence of a reduced concentration of the mTOR inhibitor or in the absence of an mTOR inhibitor) may take place for equal to or less than about 36 days, for example equal to or less than about 30 days or equal to or less than about 25 days after the concentration of the mTOR inhibitor is reduced or the mTOR inhibitor is removed. The further culture or expansion step (in the presence of a reduced concentration of the mTOR inhibitor or in the absence of an mTOR inhibitor) may take place for equal to or less than about 20 days after the concentration of the mTOR inhibitor is reduced or the mTOR inhibitor is removed. For example, this further culture or expansion step may take place for equal to or less than about 19 days or equal to or less than about 18 days or equal to or less than about 17 days or equal to or less than about 16 days or equal to or less than about 15 days or equal to or less than about 14 days after the concentration of the mTOR inhibitor is reduced or the mTOR inhibitor is removed.

The further culture or expansion step (in the presence of a reduced concentration of the mTOR inhibitor or in the absence of an mTOR inhibitor) may take place for about 6 days to about 36 days or from about 6 days to about 20 days or from about 8 days to about 20 days or from about 8 days to about 16 days or from about 8 days to about 14 days or from about 10 days to about 14 days after the concentration of the mTOR inhibitor is reduced or the mTOR inhibitor is removed.

The in vitro method described herein may further comprise a step of introducing a heterologous nucleic acid into one or more Tregs within the population. For example, the step of introducing a heterologous nucleic acid into the Tregs may take place after step (c). For example, the step of introducing a heterologous nucleic acid into the Tregs may take place at the same time as the step of reducing the concentration of the mTOR inhibitor. In particular, the step of introducing a heterologous nucleic acid into the Tregs may take place after the concentration of the mTOR inhibitor has been reduced or the mTOR inhibitor has been removed and may therefore take place in the presence of a reduced concentration of the mTOR inhibitor or in the absence of an mTOR inhibitor.

It may be desirable to introduce the heterologous nucleic acid into as many Tregs as possible, however the heterologous nucleic acid may not be introduced into all of the Tregs within the population and it is not a requirement of the method or this step of the method that the heterologous nucleic acid is introduced into all of the Tregs. The heterologous nucleic acid may be introduced into the other (non-Treg) cells within the population, although this is not a requirement of the method. In certain embodiments, the heterologous nucleic acid is introduced into at least about 10% of the cells (particularly Tregs) in the population (e.g. a transduction efficiency of at least about 10%), for example the heterologous nucleic acid may be introduced into at least about 15% or at least about 20% or at least about 25% or at least about 30% of cells (particularly Tregs) in the population. For example, the heterologous nucleic acid may be introduced into up to about 100% or up to about 95% or up to about 90% of the cells (particularly Tregs) in the population. In certain embodiments, the heterologous nucleic acid is introduced into at least about 10% of the Tregs (e.g. a transduction efficiency of at least about 10%), for example the heterologous nucleic acid may be introduced into at least about 15% or at least about 20% or at least about 25% or at least about 30% of the Tregs. For example, the heterologous nucleic acid may be introduced into up to about 100% or up to about 95% or up to about 90% of the Tregs. After the heterologous nucleic acid has been introduced into the Tregs, the Tregs that have been transduced may be separated from the Tregs that have not been transduced using a selectable marker encoded by the heterologous nucleic acid.

In certain embodiments, the concentration of the mTOR inhibitor used in step (c) is reduced or the mTOR inhibitor used in step (c) is removed before or when the heterologous nucleic acid is introduced into the Tregs. For example, the addition of the materials required to introduce the heterologous nucleic acid into the Tregs to the culture medium used in step (c) may result in the mTOR inhibitor used in step (c) being diluted. The concentration of the mTOR inhibitor used in step (c) may be diluted to a concentration equal to or less than about 25 nM as described above. The concentration of the mTOR inhibitor used in step (c) may be diluted by a factor of at least about 2, 3 or 4. For example, the concentration of the mTOR inhibitor used in step (c) may be diluted by a factor of at least about 5, at least about 6, at least about 7, at least about 8, at least about 9, or at least about 10. Thus, the step of introducing the heterologous nucleic acid into the Tregs may take place and/or result in a reduced concentration of an mTOR inhibitor.

Alternatively, the culture medium used in step (c) (and consequently the mTOR inhibitor used in step (c)) may be removed from the Tregs, the Tregs may optionally be washed, and the materials required to introduce the heterologous nucleic acid into the Tregs, which may be present in fresh culture medium such as XVIVO™ media or TexMACS™ media, may then be contacted with the Tregs. The materials used to introduce the heterologous nucleic acid into the Tregs may not include any mTOR inhibitor. Thus, the step of introducing the heterologous nucleic acid into the Tregs may take place in the absence of an mTOR inhibitor.

The heterologous nucleic acid may be introduced into the Tregs at least about 6 hours after the start of step (b) and/or the start of step (c). For example, the heterologous nucleic acid may be introduced into the Tregs at least about 8 hours or at least about 10 hours or at least about 12 hours or at least about 14 hours or at least about 16 hours or at least about 18 hours or at least about 20 hours or at least about 22 hours or at least about 24 hours after the start of step (b) and/or the start of step (c). In certain embodiments, the heterologous nucleic acid may be introduced into the Tregs at least about 26 hours or at least about 28 hours or at least about 30 hours or at least about 32 hours or at least about 34 hours or at least about 36 hours or at least about 38 hours or at least about 40 hours or at least about 42 hours or at least about 44 hours or at least about 46 hours or at least about 48 hours or at least about 50 hours or at least about 52 hours or at least about 54 hours after the start of step (b) and/or the start of step (c).

The heterologous nucleic acid may be introduced into the Tregs equal to or less than about 6 days after the start of step (b) and/or the start of step (c). For example, the heterologous nucleic acid may be introduced into the Tregs equal to or less than about 120 hours or equal to or less than about 96 hours or equal to or less than about 84 hours or equal to or less than about 72 hours or equal to or less than about 60 hours or equal to or less than about 48 hours after the start of step (b) and/or the start of step (c).

For example, the heterologous nucleic acid may be introduced into the Tregs about 6 hours to about 6 days or from about 12 hours to about 72 hours or from about 12 hours to about 60 hours or from about 24 hours to about 72 hours or from about 24 hours to about 60 hours or from about 36 hours to about 60 hours after the start of step (b) and/or the start of step (c). For example, the heterologous nucleic acid may be introduced into the Tregs about 24 hours or about 48 hours after the start of step (b) and/or the start of step (c).

The in vitro method may further comprise a further step of culturing or expanding the Tregs after the heterologous nucleic acid has been introduced into the Tregs. The further step of culturing or expanding the Tregs after the heterologous nucleic acid has been introduced into the Tregs may be the additional culturing or expanding step in the presence of a reduced concentration of the mTOR inhibitor or in the absence of an mTOR inhibitor, as described above.

The further step of culturing or expanding the Tregs after the heterologous nucleic acid has been introduced into the Tregs may be performed in the presence of a culture medium suitable for Treg culture. Exemplary culture media that are known to be suitable for culturing Tregs include XVIVO™ media or TexMACS™ media. The culture media may, for example, be supplemented with serum, for example human serum, particularly human AB serum.

The further step of culturing or expanding the Tregs after the heterologous nucleic acid has been introduced into the Tregs may take place for at least about 6 days after the heterologous nucleic acid is introduced into the Tregs. For example, this additional culture or expanding step may take place for at least about 7 days or at least about 8 days or at least about 9 days or at least about 10 days or at least about 11 days or at least about 12 days after the heterologous nucleic acid is introduced into the Tregs. By “after the heterologous nucleic acid is introduced into the Tregs” it is meant the after the start of the step of introducing the heterologous nucleic acid into the Tregs. In other words at least 6 days after the heterologous nucleic acid is introduced into the Tregs means at least 6 days after the materials used to introduce the heterologous nucleic acid into the Tregs are first contacted with the Tregs.

The further step of culturing or expanding the Tregs after the heterologous nucleic acid has been introduced into the Tregs may take place for equal to or less than about 36 days, for example equal to or less than about 30 days or equal to or less than about 25 days after the heterologous nucleic acid is introduced into the Tregs. The further step of culturing or expanding the Tregs after the heterologous nucleic acid has been introduced into the Tregs may take place for equal to or less than about 20 days after the heterologous nucleic acid is introduced into the Tregs. For example, this further culture or expanding step may take place for equal to or less than about 19 days or equal to or less than about 18 days or equal to or less than about 17 days or equal to or less than about 16 days or equal to or less than about 15 days or equal to or less than about 14 days after the heterologous nucleic acid is introduced into the Tregs.

The further step of culturing or expanding the Tregs after the heterologous nucleic acid has been introduced into the Tregs may take place for about 6 days to about 36 days or from about 6 days to about 20 days or from about 8 days to about 20 days or from about 8 days to about 16 days or from about 8 days to about 14 days or from about 10 days to about 14 days after the heterologous nucleic acid is introduced into the Tregs.

As used herein, the term “introduced” refers to methods for inserting foreign nucleic acid, e.g., DNA or RNA, into a cell. As used herein the term introduced includes both transduction and transfection methods. Transfection is the process of introducing nucleic acids into a cell by non-viral methods. Transduction is the process of introducing foreign DNA or RNA into a cell via a viral vector. Engineered cells may be generated by introducing a nucleic acid as described herein by one of many means including transduction with a viral vector, transfection with DNA or RNA. An “engineered cell” means a cell which has been modified to comprise or express a polynucleotide which is not naturally encoded by the cell. Methods for engineering cells are known in the art and include, but are not limited to, genetic modification of cells e.g., by transduction such as retroviral or lentiviral transduction, transfection (such as transient transfection—DNA or RNA based) including lipofection, polyethylene glycol, calcium phosphate and electroporation, as discussed herein. Any suitable method may be used to introduce a nucleic acid sequence into a cell. Non-viral technologies such as amphipathic cell penetrating peptides may be used to introduce nucleic acid.

In particular, the in vitro methods described herein may further comprise a step of transducing the Tregs with a viral vector comprising the heterologous nucleic acid.

A vector is a tool that allows or facilitates the transfer of an entity from one environment to another. As used herein, and by way of example, some vectors used in recombinant nucleic acid techniques allow entities, such as a segment of nucleic acid (e.g., a heterologous DNA segment, such as a heterologous cDNA segment), to be transferred into a target cell. Vectors may be non-viral or viral. Examples of vectors used in recombinant nucleic acid techniques include, but are not limited to, plasmids, mRNA molecules (e.g., in vitro transcribed mRNAs), chromosomes, artificial chromosomes and viruses. The vector may also be, for example, a naked nucleic acid (e.g., DNA). In its simplest form, the vector may itself be a nucleotide of interest.

The vectors used herein may be, for example, plasmid, mRNA or virus vectors and may include a promoter (as described herein) for the expression of a nucleic acid molecule/polynucleotide and optionally a regulator of the promoter.

In an embodiment the vector is a viral vector, for example a retroviral, e.g., a lentiviral vector or a gamma retroviral vector.

The vectors may further comprise additional promoters, for example, in one embodiment, the promoter may be a LTR, for example, a retroviral LTR or a lentiviral LTR. Long terminal repeats (LTRs) are identical sequences of DNA that repeat hundreds or thousands of times found at either end of retrotransposons or proviral DNA formed by reverse transcription of retroviral RNA. They are used by viruses to insert their genetic material into the host genomes. Signals of gene expression are found in LTRs: enhancer, promoter (can have both transcriptional enhancers or regulatory elements), transcription initiation (such as capping), transcription terminator and polyadenylation signal.

Suitably, the vector may include a 5′LTR and a 3′LTR.

The vector may comprise one or more additional regulatory sequences which may act pre- or post-transcriptionally. “Regulatory sequences” are any sequences which facilitate expression of the polypeptides, e.g., act to increase expression of a transcript or to enhance mRNA stability. Suitable regulatory sequences include for example enhancer elements, post-transcriptional regulatory elements and polyadenylation sites. Suitably, the additional regulatory sequences may be present in the LTR(s).

Suitably, the vector may comprise a Woodchuck Hepatitis Virus Posttranscriptional Regulatory Element (WPRE), e.g., operably linked to the promoter.

Vectors comprising the heterologous nucleic acid may be introduced into cells using a variety of techniques known in the art, such as transformation and transduction. Several techniques are known in the art, for example infection with recombinant viral vectors, such as retroviral, lentiviral, adenoviral, adeno-associated viral, baculoviral and herpes simplex viral vectors; direct injection of nucleic acids and biolistic transformation.

Non-viral delivery systems include but are not limited to DNA transfection methods. Here, transfection includes a process using a non-viral vector to deliver a gene to a target cell. Non-viral delivery systems can include liposomal or amphipathic cell penetrating peptides, preferably complexed with a nucleic acid molecule or construct.

Typical transfection methods include electroporation, DNA biolistics, lipid-mediated transfection, compacted DNA-mediated transfection, liposomes, immunoliposomes, lipofectin, cationic agent-mediated transfection, cationic facial amphiphiles (CFAs) (Nat. Biotechnol. (1996) 14: 556) and combinations thereof.

Although the present heterologous nucleic acid molecules may be designed to be used as single constructs, and this would be contained in a single vector, it is not precluded that they are introduced into a cell in conjunction with other vectors, for example encoding other polypeptides it may be desired also to introduce into the cell.

The heterologous nucleic acid that may be introduced into the Tregs in the methods described herein may comprise a sequence encoding a chimeric antigen receptor (CAR) or a TCR and/or a sequence encoding a FOXP3 polypeptide and/or a sequence encoding a safety switch and/or a sequence encoding a polypeptide that increases persistence of the cells.

The term “chimeric antigen receptor” or “CAR” as used herein refers to engineered receptors which can confer an antigen specificity onto cells (for example Tregs). CARs are also known as artificial T-cell receptors, chimeric T-cell receptors or chimeric immunoreceptors. A CAR typically comprises an extracellular domain comprising an antigen-specific targeting region, termed herein an antigen-binding domain, a transmembrane domain, and an intracellular domain comprising optionally one or more co-stimulatory domains, and an intracellular signalling domain. The antigen-binding domain is typically joined to the transmembrane domain by a hinge domain. The design of CARs, and the various domains that they may contain, is well-known in the art.

When the CAR binds its target antigen, this results in the transmission of an activating signal to the cell in which it is expressed. Thus, the CAR directs the specificity of the engineered cells towards the target antigen, particularly towards cells expressing the targeted antigen.

The antigen-binding domain of a CAR may be derived or obtained from any protein or polypeptide which binds (i.e. has affinity for) a desired target antigen, or more generally a desired target molecule. This may be, for example, a ligand or receptor, or a physiological binding protein for the target molecule, or a part thereof, or a synthetic or derivative protein. The target molecule may commonly be expressed on the surface of a cell, for example a target cell, or a cell in the vicinity of a target cell (for a bystander effect), but need not be. Depending on the nature and specificity of the antigen binding domain, the CAR may recognize a soluble molecule, for example where the antigen-binding domain is based on, or derived from, a cellular receptor.

The antigen-binding domain is most commonly derived from antibody variable chains (for example it commonly takes the form of a scFv), but may also be generated from T-cell receptor variable domains or, as mentioned above, other molecules, such as receptors for ligands or other binding molecules.

The CAR is typically expressed as a polypeptide also comprising a signal sequence (also known as a leader sequence), and in particular a signal sequence which targets the CAR to the plasma membrane of the cell. This will generally be positioned next to or close to the antigen-binding domain, generally upstream of the antigen-binding domain. The extracellular domain, or ectodomain, of the CAR may thus comprise a signal sequence and an antigen-binding domain.

The antigen-binding domain provides the CAR with the ability to bind a predetermined antigen of interest. The antigen-binding domain preferably targets an antigen of clinical interest or an antigen at a site of disease.

As noted above, the antigen-binding domain may be any protein or peptide that possesses the ability to specifically recognize and bind to a biological molecule (e.g., a cell surface receptor or a component thereof). The antigen-binding domain includes any naturally occurring, synthetic, semi-synthetic, or recombinantly produced binding partner for a biological molecule of interest. Illustrative antigen-specific targeting domains include antibodies or antibody fragments or derivatives, extracellular domains of receptors, ligands for cell surface molecules/receptors, or receptor binding domains thereof, and tumor binding proteins. Although as discussed below, the antigen-specific targeting domain may preferably be an antibody or derived from an antibody, other antigen-specific targeting domains are encompassed, e.g. antigen-specific targeting domains formed from an antigenic peptide/MHC or HLA combination which is capable of binding to the TCRs of Tcon cells active at a site of transplantation, inflammation or disease.

The CAR may be directed towards any desired target antigen or molecule. This may be selected according to the intended therapy, and the condition it is desired to treat. It may for example be an antigen or molecule associated with a particular condition, or an antigen or molecule associated with a cell it is desired to target to treat the condition. Typically, the antigen or molecule is a cell-surface antigen or molecule.

The term “directed against” is synonymous with “specific for” or “anti”. Put another way, the CAR recognises a target molecule. Accordingly, it is meant that the CAR is capable of binding specifically to a specified or given antigen, or target. In particular, the antigen-binding domain of the CAR is capable of binding specifically to the target molecule or antigen (more particularly when the CAR is expressed on the surface of a cell, notably an immune effector cell). Specific binding may be distinguished from non-specific binding to a non-target molecule or antigen. Thus, a cell expressing the CAR is directed, or re-directed, to bind specifically to a target cell, expressing the target molecule or antigen, particularly a target cell expressing the target antigen or molecule on its cell surface.

Antigens which may be targeted by the present CAR include, but are not limited to, antigens expressed on cells associated with transplanted organs, autoimmune diseases, allergic diseases and inflammatory diseases (e.g. neurodegenerative disease). It will be understood by a skilled person that where the cell engineered to express the CAR is a Treg cell, or a precursor therefor, due to the bystander effect of Treg cells, the antigen may be simply present and/or expressed at the site of transplantation, inflammation or disease.

Antigens expressed on cells associated with neurodegenerative disease include those presented on glial cells, e.g. MOG.

Antigens associated with organ transplants and/or cells associated with transplanted organs include, but are not limited to, a HLA antigen present in the transplanted organ but not in the patient, or an antigen whose expression is up-regulated during transplant rejection such as CCL19, MMP9, SLC1A3, MMP7, HMMR, TOP2A, GPNMB, PLA2G7, CXCL9, FABP5, GBP2, CD74, CXCL10, UBD, CD27, CD48, CXCL11.

In an embodiment the CAR is directed against an HLA antigen, and in particular an HLA-A2 antigen.

Antibodies against such antigens and are known in the art, and conveniently a scFv may be obtained or generated based on a known or available antibody. In this regard VH and VL, and CDR sequences are publicly available to aid the preparation of such an antibody-binding domain, for example in WO 2020/044055, the disclosure of which is herein incorporated by reference. Any of the antigen binding domains, or CDR, VH, and/or VL sequences disclosed in WO 2020/044055 or WO 2020/201230 may be used.

By way of example, the CAR may comprise an antigen binding domain which is capable of binding HLA-A2 (HLA-A2 may also be referred to herein as HLA-A*02, HLA-A02, and HLA-A*2). HLA-A*02 is one particular class I major histocompatibility complex (MHC) allele group at the HLA-A locus.

The antigen recognition domain may bind, suitably specifically bind, one or more regions or epitopes within HLA-A2. An epitope, also known as antigenic determinant, is the part of an antigen that is recognised by an antigen recognition domain (e.g. an antibody). In other words, the epitope is the specific piece of the antigen to which an antibody binds. Suitably, the antigen recognition domain binds, suitably specifically binds, to one region or epitope within HLA-A2.

The heterologous nucleic acid may, for example, comprise a sequence encoding a safety switch polypeptide. A safety switch polypeptide provides a cell in or on which it is expressed with a suicide moiety. This is a useful safety mechanism which allows a cell which has been administered to a subject to be deleted should the need arise, or indeed more generally, according to desire or need, for example once a cell has performed or completed its therapeutic effect.

A suicide moiety possesses an inducible capacity to lead to cell death, or more generally to elimination or deletion of a cell. An example of a suicide moiety is a suicide protein, encoded by a suicide gene, which may be expressed in or on a cell alongside a desired transgene, which when expressed allows the cell to be deleted to turn off expression of the transgene. A suicide moiety herein is a suicide polypeptide that is a polypeptide that under permissive conditions, namely conditions that are induced or turned on, is able to cause the cell to be deleted.

The suicide moiety may be a polypeptide, or amino acid sequence, which may be activated to perform a cell-deleting activity by an activating agent which is administered to the subject, or which is active to perform a cell-deleting activity in the presence of a substrate which may be administered to a subject. In a particular embodiment, the suicide moiety may represent a target for a separate cell-deleting agent which is administered to the subject. By binding to the suicide moiety, the cell-deleting agent may be targeted to the cell to be deleted. In particular, the suicide moiety may be recognised by an antibody, and binding of the antibody to the safety switch polypeptide, when expressed on the surface of a cell, cause the cell to be eliminated or deleted.

The suicide moiety may be HSV-TK or iCasp9 as is known in the art. However, in other examples the suicide moiety may be, or may comprise an epitope which is recognised by a cell-deleting antibody or other binding molecule capable of eliciting deletion of the cell. The term “delete” as used herein in the context of cell deletion is synonymous with “remove” or “ablate” or “eliminate”. The term is used to encompass cell killing, or inhibition of cell proliferation, such that the number of cells in the subject may be reduced. 100% complete removal may be desirable but may not necessarily be achieved. Reducing the number of cells, or inhibiting their proliferation, in the subject may be sufficient to have a beneficial effect.

In particular, the suicide moiety may be a CD20 epitope which is recognised by the antibody Rituximab. Thus, in the safety switch polypeptide the suicide moiety may comprise a minimal epitope based on the epitope from CD20 that is recognised by the antibody Rituximab. More particularly, the polypeptide may comprise two CD20 epitopes R1 and R2 that are spaced apart by a linker L.

Safety switches based on Rituximab epitopes are described in WO 2013/15339 and WO 2021/239812, the contents of which are incorporated herein by reference. Peptides which mimic the epitope recognised by Rituximab (so-called mimitopes) have been developed, and these were used in WO 2013/15339 as a suicide moiety in a combined suicide-marker polypeptide construct also comprising a CD34 minimal epitope as a marker moiety. Specifically, WO 2013/15339 discloses a polypeptide termed RQR8, which comprises two CD20 minimal epitopes, separated from one another by spacer sequences and an intervening CD34 marker sequence, and further linked to a stalk sequence which allows the polypeptide to project from the surface of a cell on which it is expressed. The safety switch polypeptide may be RQR8 or a variant thereof having at least about 80% sequence identity thereto, e.g. at least about 85, 88, 90, 95, 96, 97, 98, or 99% sequence identity thereto. Other safety switch polypeptides which may be used as the basis of safety switch domains include those described in our co-pending PCT patent application No. PCT/EP2021/064053 (WO 2021/239812), the contents of which are incorporated herein by reference.

FOXP3 is the abbreviated name of the transcription factor forkhead box P3 protein. Expression of heterologous FOXP3 may assist in increasing FOXP3 expression within the cell and maintaining the suppressive phenotype of Treg cells or cells with a regulatory phenotype. FOXP3 is a member of the FOX protein family of transcription factors and functions as a master regulator of the regulatory pathway in the development and function of regulatory T-cells (Tregs). “FOXP3” as used herein encompasses variants, isoforms, and functional fragments of FOXP3. A “FOXP3 polypeptide” is a polypeptide having FOXP3 activity, i.e. a polypeptide able to bind FOXP3 target DNA and function as a transcription factor regulating development and function of Tregs.

“Increasing FOXP3 expression” means to increase the levels of FOXP3 mRNA and/or protein in a cell (or population of cells) in comparison to a corresponding cell which has not been modified (or population of cells) by introduction of the nucleic acid molecule or vector. For example, the level of FOXP3 mRNA and/or protein in a cell modified according to the present invention (or a population of such cells) may be increased to at least 1.5-fold, at least 2-fold, at least 5-fold, at least 10-fold, at least 50-fold, at least 100-fold, at least 150-fold greater than the level in a corresponding cell which has not been modified according to the present invention (or population of such cells).

Suitably, the level of FOXP3 mRNA and/or protein in a modified cell (or a population of such cells) may be increased to at least 1.5-fold greater, 2-fold greater, or 5-fold greater than the level in a corresponding cell which has not been so modified (or population of such cells).

Techniques for measuring the levels of specific mRNA and protein are well known in the art. mRNA levels in a population of cells, such as Tregs, may be measured by techniques such as the Affymetrix ebioscience prime flow RNA assay, Northern blotting, serial analysis of gene expression (SAGE) or quantitative polymerase chain reaction (qPCR). Protein levels in a population of cells may be measured by techniques such as flow cytometry, high-performance liquid chromatography (HPLC), liquid chromatography-mass spectrometry (LC/MS), Western blotting or enzyme-linked immunosorbent assay (ELISA).

A “FOXP3 polypeptide” is a polypeptide having FOXP3 activity i.e., a polypeptide able to bind FOXP3 target DNA and function as a transcription factor regulating development and function of Tregs. Particularly, a FOXP3 polypeptide may have the same or similar activity to wildtype FOXP3, e.g., may have at least 40, 50, 60, 70, 80, 90, 95, 100, 110, 120, 130, 140 or 150% of the activity of the wildtype FOXP3 polypeptide. Thus, a FOXP3 polypeptide encoded by the nucleotide sequence in the nucleic acid or vector described herein may have increased or decreased activity compared to wildtype FOXP3. Techniques for measuring transcription factor activity are well known in the art. For example, transcription factor DNA-binding activity may be measured by ChIP. The transcription regulatory activity of a transcription factor may be measured by quantifying the level of expression of genes which it regulates. Gene expression may be quantified by measuring the levels of mRNA and/or protein produced from the gene using techniques such as Northern blotting, SAGE, qPCR, HPLC, LC/MS, Western blotting or ELISA. Genes regulated by FOXP3 include cytokines such as IL-2, IL-4 and IFN-γ (Siegler et al. Annu. Rev. Immunol. 2006, 24: 209-26, incorporated herein by reference). As discussed in detail below, FOXP3 or a FOXP3 polypeptide includes functional fragments, variants, and isoforms thereof.

A “functional fragment of FOXP3” may refer to a portion or region of a FOXP3 polypeptide or a polynucleotide (i.e., nucleotide sequence) encoding a FOXP3 polypeptide that has the same or similar activity to the full-length FOXP3 polypeptide or polynucleotide. The functional fragment may have at least 40%, at least 50%, at least 60%, at least 70%, at least 80%, at least 90%, at least 95%, or 100% of the activity of the full-length FOXP3 polypeptide or polynucleotide. A person skilled in the art would be able to generate functional fragments based on the known structural and functional features of FOXP3. These are described, for instance, in Song, X., et al., 2012. Cell reports, 1(6), pp. 665-675; Lopes, J. E., et al., 2006. The Journal of Immunology, 177(5), pp. 3133-3142; and Lozano, T., et al, 2013. Frontiers in oncology, 3, p. 294. Further, a N and C terminally truncated FOXP3 fragment is described within WO2019/241549 (incorporated herein by reference).

A “FOXP3 variant” may include an amino acid sequence or a nucleotide sequence which may be at least 50%, at least 55%, at least 65%, at least 70%, at least 75%, at least 80%, at least 85% or at least 90% identical, preferably at least 95% or at least 97% or at least 99% identical to a FOXP3 polypeptide or a polynucleotide encoding a FOXP3 polypeptide (e.g. wildtype FOXP3). FOXP3 variants may have the same or similar activity to a wildtype FOXP3 polypeptide or polynucleotide, e.g., may have at least 40, 50, 60, 70, 80, 90, 95, 100, 110, 120, 130, 140 or 150% of the activity of a wildtype FOXP3 polypeptide or polynucleotide. A person skilled in the art would be able to generate FOXP3 variants based on the known structural and functional features of FOXP3 and/or using conservative substitutions. FOXP3 variants may have similar or the same turnover time (or degradation rate) within a Treg cell as compared to wildtype FOXP3, e.g., at least 40, 50, 60, 70, 80, 90, 95, 99 or 100% of the turnover time (or degradation rate) of wildtype FOXP3 in a Treg. Some FOXP3 variants may have a reduced turnover time (or degradation rate) as compared to wildtype FOXP3, for example, FOXP3 variants having amino acid substitutions at amino acid 418 and/or 422 of wildtype FOXP3, for example S418E and/or S422A, as described in WO2019/241549 (incorporated herein by reference), which represent the aa418, aa422 and aa418 and aa422 mutants respectively.

Suitably, the FOXP3 polypeptide encoded by a nucleic acid molecule or vector as described herein may comprise or consist of the polypeptide sequence of a human wildtype FOXP3, such as UniProtKB accession Q9BZS1, or a functional fragment or variant thereof.

In some embodiments of the invention, the FOXP3 polypeptide comprises or consists of an amino acid sequence which is at least 70% identical to human wildtype FOXP3 or a functional fragment thereof. Suitably, the FOXP3 polypeptide comprises or consists of an amino acid sequence which is at least 80%, at least 85%, at least 90%, at least 95%, at least 98% or at least 99% identical to human wildtype FOXP3 or a functional fragment thereof. In some embodiments, the FOXP3 polypeptide comprises or consists of human wildtype FOXP3 or a functional fragment thereof.

In some embodiments, as discussed above, the FOXP3 polypeptide may comprise mutations at residues 418 and/or 422 of human wildtype FOXP3.

In some embodiments of the invention, the FOXP3 polypeptide may be truncated at the N and/or C terminal ends, resulting in the production of a functional fragment.

Suitably, the FOXP3 polypeptide may be a variant of human wildtype FOXP3, for example a natural variant. Suitably, the FOXP3 polypeptide is an isoform of human wildtype FOXP3. For example, the FOXP3 polypeptide may comprise a deletion of amino acid positions 72-106 relative to human wildtype FOXP3. Alternatively, the FOXP3 polypeptide may comprise a deletion of amino acid positions 246-272 relative to human wildtype FOXP3.

A skilled person will appreciate that FOXP3 expression within a Treg may be increased indirectly by introducing a polynucleotide into the cell which encodes a protein which increases transcription and/or translation of FOXP3 or which increases the half life (e.g. by at least 10, 20, 30, 40, 50, 60, 70, 80 or 90%) or function of FOXP3 (e.g. determined by suppressive ability of a transduced Treg, measured as previously discussed). For example, it may be possible to introduce a polynucleotide into a Treg which increases transcription of endogenous FOXP3 by interacting with the endogenous FOXP3 promoter or non-coding sequences (CNS, e.g. CNS1, 2 or 3) which are found upstream of the coding region.

The heterologous nucleic acid may comprise a sequence encoding a polypeptide that increases the persistence of the cells, for example to provide a productive IL signal to the cell without requiring exogenous IL to be administered. Such IL signal may be constitutive or inducible. Exemplary technologies may, for example, involve the use of engineered or chimeric receptors that can transmit an IL signal without requiring exogenous IL to be administered. For example, inducible engineered receptors such as those described in WO 2018/111834, WO 2019/169290 and WO 2020/264039; or constitutive engineered receptors such as those described in WO 2018/038954, WO 2019/102207, WO 2019/053420, WO 2020/180694 and WO 2017/218850; chimeric cytokine receptors such as those described in WO 2020/183131, WO 2017/029512, WO 2012/138858, WO 2014/172584, WO 2017/068360, WO 2021/023987, WO 2020/180664 and WO 2020/044239; or engineered receptors having a tethered activation molecule such as those described in WO 2017/201432 and WO 2019/183389.

The heterologous nucleic acid may further comprise one or more other coding sequences which may encode a protein of interest, for example a therapeutic protein. The one or more additional nucleotide sequences may, for example, encode transcription factors, growth factors or other factors which may assist in enhancing the functionality or survival of the cell. The one or more additional nucleotide sequences may, for example, encode an additional receptor, particularly an antigen receptor such as a heterologous T-cell receptor (TCR) or a derivative thereof (e.g. a TCR-CAR construct, or single chain TCR construct etc.)

The heterologous nucleic acid may further encode a selectable marker. Suitable selectable markers are well-known in the art and include, but are not limited to, fluorescent proteins such as GFP. Suitably, the selectable marker may be a fluorescent protein, for example GFP, YFP, RFP, tdTomato, dsRed, or variants thereof. In some embodiments, the fluorescent protein is GFP or a GFP variant. Suitably, the selectable marker/reporter domain may be a luciferase-based reporter, a PET reporter (e.g Sodium Iodide Symporter (NIS)), or a membrane protein (e.g. CD34, low-affinity nerve growth factor (LNGFR)).

The use of a selectable marker is advantageous as it allows cells (e.g. Tregs) in which a nucleic acid has been successfully introduced to be selected and isolated from a starting cell population using common methods, e.g. flow cytometry.

Suitably, the heterologous nucleic acid may be codon optimised. Suitably, the heterologous nucleic acid may be codon optimised for expression in a human cell. Codon optimisation has previously been described in WO1999/41397 and WO2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

Where two or more coding sequences are expressed from a single nucleic acid molecule, they may be linked by a sequence allowing co-expression of the two or more coding sequences. In particular, the co-expression sequence, or alternatively termed, the co-expression site, may enable expression of an encoded protein or polypeptide as a discrete entity. For example, the nucleic acid may comprise an internal promoter, an internal ribosome entry sequence (IRES) sequence or a sequence encoding a cleavage site.

In particular, the co-expression sequence may encode a self-cleavage sequence in between encoded polypeptides. Particularly, the self-cleaving sequence may be a self-cleaving peptide. Such sequences auto-cleave during protein production. Self-cleaving peptides which may be used are 2A peptides or 2A-like peptides which are known and described in the art, for example in Donnelly et al., Journal of General Virology, 2001, 82, 1027-1041, herein incorporated by reference. 2A and 2A-like peptides are believed to cause ribosome skipping, and result in a form of cleavage in which a ribosome skips the formation of peptide bond between the end of a 2A peptide and the downstream amino acid sequence. The “cleavage” occurs between the Glycine and Proline residues at the C-terminus of the 2A peptide meaning the upstream cistron will have a few additional residues added to the end, while the downstream cistron will start with the Proline.

Suitable self-cleaving domains include P2A, T2A, E2A, and F2A sequences. The sequences may be modified to include the amino acids GSG at the N-terminus of the 2A peptides. Such modified alternative 2A sequences are known and reported in the art. Alternative 2A-like sequences which may be used are shown in Donnelly et al (supra), for example a TaV sequence.

The self-cleaving sequences included in the nucleic acid may be the same or different.

The self-cleaving sequence may include an additional cleavage site, which may be cleaved by common enzymes present in the cell. This may assist in achieving complete removal of the 2A sequences after translation. Such an additional cleavage site may for example comprise a Furin cleavage site.

Heterologous nucleic acid molecules and polynucleotides/nucleic acid sequences as defined herein may comprise DNA or RNA. They may be single-stranded or double-stranded. It will be understood by a skilled person that numerous different nucleic acid molecules/polynucleotides can encode the same polypeptide as a result of the degeneracy of the genetic code. In addition, it is to be understood that the skilled person may, using routine techniques, make nucleotide substitutions that do not affect the polypeptide sequence encoded by the nucleic acid molecules/polynucleotides/nucleotide sequences as defined herein to reflect the codon usage of any particular host organism in which the polypeptides of the invention are to be expressed.

The heterologous nucleic acids may be modified by any method available in the art. Such modifications may be carried out in order to enhance the in vivo activity or lifespan of the nucleic acid molecules as defined herein.

Nucleic acid molecules such as DNA nucleic acid molecules may be produced recombinantly, synthetically or by any means available to those of skill in the art. They may also be cloned by standard techniques.

Longer nucleic acid molecules/polynucleotides/nucleotide sequences will generally be produced using recombinant means, for example using polymerase chain reaction (PCR) cloning techniques. This will involve making a pair of primers (e.g. of about 15 to 30 nucleotides) flanking the target sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture with an agarose gel) and recovering the amplified DNA. The primers may be designed to contain suitable restriction enzyme recognition sites so that the amplified DNA can be cloned into a suitable vector.

The heterologous nucleic acid may comprise one or more regulatory sequences, for example a promoter. A “promoter” is a region of DNA that leads to initiation of transcription of a gene. Promoters are located near the transcription start sites of genes, upstream on the DNA (towards the 5′ region of the sense strand). Any suitable promoter may be used, the selection of which may be readily made by the skilled person. The promoter may be from any source, and may be a viral promoter, or a eukaryotic promoter, including mammalian or human promoters (i.e. a physiological promoter). In an embodiment the promoter is a viral promoter. Particular promoters include LTR promoters, EFS (or functional truncations thereof), SFFV, PGK, and CMV. In an embodiment the promoter is SFFV or a viral LTR promoter. Particularly, a SFFV promoter may be used within a nucleic acid molecule to allow initiation of transcription of the nucleotide sequence(s). Where there is more than one nucleotide sequence, each sequence may be operably linked to the same promoter, e.g. nucleotide sequences encoding the CAR, FOXP3, safety switch, and/or polypeptide increasing the persistence of the cells.

“Operably linked to the same promoter” means that transcription of the polynucleotide sequences may be initiated from the same promoter (e.g., transcription of the first, second and third polynucleotide sequences is initiated from the same promoter) and that the nucleotide sequences are positioned and oriented for transcription to be initiated from the promoter. Polynucleotides operably linked to a promoter are under transcriptional regulation of the promoter.

The in vitro method described herein may further comprise a step of harvesting a population of Tregs. By “harvesting”, it is meant any step to isolate the product of the in vitro method described herein, which is a population of Tregs, such that the product is in a form suitable for ACT. This may, for example, involve removing culture medium and/or removing any TCR/CD3 activator and TCR co-stimulator activator from the population of Tregs and/or further steps to remove any impurities in the product. The harvesting step may be the last step of the in vitro method described herein. Alternatively, harvesting followed by cryopreserving (as described below) may be the last two steps of the in vitro method described herein.

The population of Tregs may be harvested from about 8 days to about 36 days after the start of step (b). For example, the population of Tregs may be harvested from about 8 days to about 30 days or from about 8 days to about 25 days or from about 8 days to about 22 days after the start of step (b). For example, the population of Tregs may be harvested from about 10 days to about 18 days or from about 12 days to about 16 days after the start of step (b). The population of Tregs may, for example, be cryopreserved after they have been harvested.

The product of the in vitro method described herein, which is a population of Tregs may, for example, be cryopreserved such that they can be stored prior to ACT. By “cryopreserve” or “cryopreservation” it is meant freezing the population of Tregs under conditions where the cells (particularly the Tregs) remain viable (e.g. during freezing and after any subsequent thawing). Viability of cells can be measured by any well-known method of the art, for example flow cytometry using a live/dead stain (e.g. LIVE/DEAD™ Fixable Near-IR-Dead Cell Stain (Thermofisher). Typically, at least 50%, 60%, 70%, 80%, 90% or 95% of cells (particularly at least 50%, 60%, 70%, 80%, 90% or 95% of Tregs) will remain viable during and after cryopreservation. Viability thus refers to live cells. A skilled person will appreciate that cryopreservation may allow cells to remain viable due to the application of one or more conditions which can effectively stop cell death and which can maintain the structure of the cell (for example, the use of a particular temperature, freezing and/or thawing rate and/or cryopreservant). Such conditions are known in the art. The cryopreservation step may be the last step of the in vitro method described herein.

In an in vitro method for culturing or expanding Tregs, it may be necessary for the Tregs to be contacted with interleukin-2 (IL-2) at some point during the culturing or expanding. IL-2 may or may not be present at the same time the mTOR inhibitor is present.

In certain embodiments, in the in vitro methods described herein, step (a) and/or step (b) and/or step (c) may occur in the absence of IL-2. In certain embodiments, IL-2 may be added to the culture medium after step (c). For example, IL-2 may be added to the culture medium at the same time a heterologous nucleic acid is introduced into the Tregs (i.e. at the same time the materials required to introduce a heterologous nucleic acid into the cells are first contacted with the Tregs). For example, IL-2 may be added to the culture medium at the same time or after the concentration of the mTOR inhibitor used in step (c) is reduced or at the same time or after the mTOR inhibitor used in step (c) is removed (as described above). The steps of reducing the concentration of mTOR inhibitor or removing mTOR inhibitor and introducing a heterologous nucleic acid into the Tregs are described above.

The IL-2 may be contacted with the Tregs within the population (e.g. by adding to culture medium) at least about 6 hours after the start of step (b) and/or the start of step (c). For example, IL-2 may be contacted with the Tregs within the population at least about 8 hours or at least about 10 hours or at least about 12 hours or at least about 14 hours or at least about 16 hours or at least about 18 hours or at least about 20 hours or at least about 22 hours or at least about 24 hours after the start of step (b) and/or the start of step (c). In certain embodiments, the IL-2 may be contacted with the Tregs within the population least about 26 hours or at least about 28 hours or at least about 30 hours or at least about 32 hours or at least about 34 hours or at least about 36 hours or at least about 38 hours or at least about 40 hours or at least about 42 hours or at least about 44 hours or at least about 46 hours or at least about 48 hours or at least about 50 hours or at least about 52 hours or at least about 54 hours after the start of step (b) and/or the start of step (c).

Alternatively, the IL-2 may be contacted with the Tregs within the population at the start of step (a) and/or at the start of step (b) and/or at the start of step (c).

The IL-2 may be contacted with the Tregs equal to or less than about 6 days after the start of step (b) and/or the start of step (c). For example, the IL-2 may be contacted with the Tregs equal to or less than about 120 hours or equal to or less than about 96 hours or equal to or less than about 84 hours or equal to or less than about 72 hours or equal to or less than about 60 hours or equal to or less than about 48 hours after the start of step (b) and/or the start of step (c).

For example, the IL-2 may be contacted with the Tregs about 6 hours to about 6 days or from about 12 hours to about 72 hours or from about 12 hours to about 60 hours or from about 24 hours to about 72 hours or from about 24 hours to about 60 hours or from about 36 hours to about 60 hours after the start of step (b) and/or the start of step (c).

IL-2 may be used in a concentration of from about 10 IU/mL to about 1×10⁶ IU/mL. For example, IL-2 may be used in a concentration of from about 50 IU/mL to about 1×10⁵ IU/mL or from about 100 IU/mL to about 1×10⁴ IU/mL or from about 150 IU/mL to about 5000 IU/mL or from about 200 IU/mL to about 2500 IU/mL or from about 250 IU/mL to about 1500 IU/mL. An exemplary source of IL-2 is Proleukin (Clinigen Healthcare). International Units (IU) for IL-2 are calculated based on the international standard 86-500 which can be obtained from the National Institute for Biological Standards and Control (NIBSC).

IL-2 may be further replenished at various timepoints throughout the culture (or expansion) steps as required. For example, additional IL-2 may be added at intervals of between about 12 hours and about 72 hours, for example between about 24 hours and about 72 hours, for example between about 48 hours and about 72 hours.

For example, additional IL-2 may be added to the culture medium every two to three days.

The in vitro methods described herein may comprise one or more additional activation steps (in addition to step (b)). The Tregs may be “reactivated” by any method for activation as described above in relation to step (b). For example, the Tregs may be reactivated by contacting them with a TCR/CD3 activator and/or a TCR co-stimulator activator as described above in relation to step (b). For example, additional TCR/CD3 activator and/or TCR co-stimulator activator (in addition to that originally used in step (b)) may be contacted with the Tregs, for example by adding to the culture medium. Part of the original TCR/CD3 activator and/or TCR co-stimulator activator may remain in contact with the Tregs. In other words it is not necessary to remove the TCR/CD3 activator and/or TCR co-stimulator activator used in step (b) before any additional activation steps. Where the TCR/CD3 activator and/or TCR co-stimulator activator are antibodies (which may be present on the surface of beads), the cells may internalise the antibodies. Additional TCR/CD3 activator and/or TCR co-stimulator may be added during the “reactivation” step in order to provide a suitable ratio of antibody (or beads) to cells.

The Tregs may, for example, be reactivated between 4 and 12 days after the start of step (b). For example, the Tregs may be reactivated between 6 and 10 days after the start of step (b). The method may further comprise one or more further reactivation steps after this reactivation step. Any further reactivation steps may take place between 4 and 12 days after the previous reactivation step, for example between 6 and 10 days after any previous reactivation step. The number of reactivation steps required will depend on how long the cells are kept in culture before harvesting. In certain embodiments, the method does not include more than 1 reactivation step (2 activation steps in total).

### Final Product

There is further provided herein a product obtained by and/or obtainable by the in vitro method described herein, including any embodiment thereof. The product is a population of cells comprising Tregs. The product thus comprises a population of Tregs.

The product may, for example comprise at least about 50×10⁶ cells. For example, the product may comprise at least about 100×10⁶ or at least about 150×10⁶ or at least about 200×10⁶ cells. For example, the product may comprise up to about 80×10⁸ or up to about 75×10⁸ or up to about 70×10⁸ cells.

The product may, for example comprise at least about 10×10⁶ Tregs. For example, the product may comprise at least about 15×10⁶ or at least about 20×10⁶ or at least about 25×10⁶ or at least about 30×10⁶ or at least about 35×10⁶ or at least about 40×10⁶ or at least about 45×10⁶ or at least about 50×10⁶ or at least about 55×10⁶ or at least about 60×10⁶ or at least about 75×10⁶ or at least about 80×10⁶ or at least about 85×10⁶ or at least about 90×10⁶ or at least about 95×10⁶ or at least about 100×10⁶ or at least about 150×10⁶ or at least about 200×10⁶ or at least about 250×10⁶ or at least about 300×10⁶ or at least about 350×10⁶ or at least about 400×10⁶ or at least about 450×10⁶ or at least about 500×10⁶ Tregs. For example, the product may comprise up to about 80×10⁸ or up to about 75×10⁸ or up to about 70×10⁸ Tregs.

Where a heterologous nucleic acid has been introduced into the Tregs, the product may, for example comprise at least about 10×10⁶ Tregs comprising the heterologous nucleic acid. For example, the product may comprise at least about 15×10⁶ or at least about 20×10⁶ or at least about 25×10⁶ or at least about 30×10⁶ or at least about 35×10⁶ or at least about 40×10⁶ or at least about 45×10⁶ or at least about 50×10⁶ or at least about 55×10⁶ or at least about 60×10⁶ or at least about 75×10⁶ or at least about 80×10⁶ or at least about 85×10⁶ or at least about 90×10⁶ or at least about 95×10⁶ or at least about 100×10⁶ Tregs comprising the heterologous nucleic acid. For example, the product may comprise up to about 800×10⁶ or up to about 750×10⁶ or up to about 700×10⁶ Tregs comprising the heterologous nucleic acid.

Where a heterologous nucleic acid encoding a CAR has been introduced into the Tregs, the product may, for example comprise at least about 10×10⁶ CAR-Tregs. For example, the product may comprise at least about 15×10⁶ or at least about 20×10⁶ or at least about 25×10⁶ or at least about 30×10⁶ or at least about 35×10⁶ or at least about 40×10⁶ or at least about 45×10⁶ or at least about 50×10⁶ or at least about 55×10⁶ or at least about 60×10⁶ or at least about 75×10⁶ or at least about 80×10⁶ or at least about 85×10⁶ or at least about 90×10⁶ or at least about 95×10⁶ or at least about 100×10⁶ CAR-Tregs. For example, the product may comprise up to about 800×10⁶ or up to about 750×10⁶ or up to about 700×10⁶ CAR-Tregs.

The product may, for example, comprise at least about 75% of Tregs (alternatively put, at least about 75% of the cells in the product are Tregs). For example, the product may comprise at least about 80% or at least about 85% or at least about 90% or at least about 95% of Tregs. For example, the product may comprise equal to or less than about 99% or equal to or less than about 98% or equal to or less than about 97% or equal to or less than about 96% or equal to or less than about 95% of Tregs. For example, the product may comprise from about 75% to about 99% or from about 75% to about 95% or from about 80% to about 95% of Tregs.

The product may, for example, comprise at least about 75% of cells expressing FOXP3 (alternatively put, at least about 75% of the cells in the product express FOXP3). For example, the product may comprise at least about 80% or at least about 85% or at least about 90% or at least about 95% of cells expressing FOXP3. For example, the product may comprise equal to or less than about 99% or equal to or less than about 98% or equal to or less than about 97% or equal to or less than about 96% or equal to or less than about 95% of cells expressing FOXP3. For example, the product may comprise from about 75% to about 99% or from about 75% to about 95% or from about 80% to about 95% of cells expressing FOXP3.

The product may, for example, comprise at least about 75% FOXP3+ Tregs (alternatively put, equal to or greater than about 75% of the cells in the product are FOXP3+ Tregs). For example, the product may comprise at least about 80% or at least about 85% or at least about 90% or at least about 95% FOXP3+ Tregs. For example, the product may comprise equal to or less than about 99% or equal to or less than about 98% or equal to or less than about 97% or equal to or less than about 96% or equal to or less than about 95% FOXP3+ Tregs. For example, the product may comprise from about 75% to about 99% or from about 75% to about 95% or from about 80% to about 95% FOXP3+ Tregs. Tregs may have any Treg phenotype as described herein, for example CD4+CD25+FOXP3+.

The product may, for example, comprise at least about 2% CD45RA+ Tregs (e.g. CD4+CD25+CD45RA+ or CD4+CD25+CD127−/lowCD45RA+ Tregs) (alternatively put, equal to or greater than about 2% of the cells in the product are CD45RA+ Tregs). For example, the product may comprise at least about 3% or at least about 4% or at least about 5% or at least about 6% or at least about 7% or at least about 8% or at least about 9% or at least about 10% CD45RA+ Tregs (e.g. CD4+CD25+CD45RA+ or CD4+CD25+CD127−/lowCD45RA+ Tregs). For example, the product may comprise equal to or less than about 50% or equal to or less than about 45% or equal to or less than about 40% or equal to or less than about 35% or equal to or less than about 30% or equal to or less than about 25% CD45RA+ Tregs (e.g. CD4+CD25+CD45RA+ or CD4+CD25+CD127−/lowCD45RA+ Tregs). For example, the product may comprise from about 2% to about 50% or from about 3% to about 40% or from about 4% to about 30% or from about 5% to about 20% CD45RA+ Tregs (e.g. CD4+CD25+CD45RA+ or CD4+CD25+CD127−/lowCD45RA+ Tregs). Tregs may have any Treg phenotype as described herein, for example CD4+CD25+FOXP3+.

The product may, for example, comprise at least about 60% Helios+ Tregs (alternatively put, at least about 60% of the cells in the product are Helios+ Tregs). For example, the product may comprise at least about 65% or at least about 70% or at least about 75% or at least about 80% Helios+ Tregs. For example, the product may comprise equal to or less than about 99% or equal to or less than about 98% or equal to or less than about 97% or equal to or less than about 96% or equal to or less than about 95% Helios+ Tregs. For example, the product may comprise from about 60% to about 99% or from about 65% to about 95% or from about 70% to about 95% Helios+ Tregs. Tregs may have any Treg phenotype as described herein, for example CD4+CD25+FOXP3+.

The product may, for example, comprise at least about 50% CD27+ Tregs (alternatively put, at least about 50% of the cells in the product express CD27). For example, the product may comprise at least about 55% or at least about 60% or at least about 65% or at least about 70% or at least about 75% or at least about 80% CD27+ Tregs. For example, the product may comprise equal to or less than about 99% or equal to or less than about 98% or equal to or less than about 97% or equal to or less than about 96% or equal to or less than about 95% CD27+ Tregs. For example, the product may comprise from about 50% to about 99% or from about 55% to about 95% or from about 60% to about 95% CD27+ Tregs. Tregs may have any Treg phenotype as described herein, for example CD4+CD25+FOXP3+.

The product may, for example, comprise at least about 40% demethylated FOXP3-TSDR. For example, the product may comprise at least about 45% or at least about 50% or at least about 55% or at least about 60% or at least about 65% or at least about 70% demethylated FOXP3-TSDR. For example, the product may comprise equal to or less than about 99% or equal to or less than about 98% or equal to or less than about 97% or equal to or less than about 96% or equal to or less than about 95% demethylated FOXP3-TSDR. For example, the product may comprise from about 40% to about 99% or from about 50% to about 95% or from about 60% to about 95% demethylated FOXP3-TSDR.

The product may, for example, comprise equal to or less than about 5% of CD8+ cells (alternatively put, equal to or less than about 5% of cells in the product are CD8+ cells). For example, the product may comprise equal to or less than about 4% or equal to or less than about 3% or equal to or less than about 2% or equal to or less than about 1% CD8+ cells. For example, the product may comprise from about 0.1% to about 5% or from about 0.5% to about 4% of CD8+ cells.

The product may, for example, comprise equal to or less than about 5% of CD4+CD25− cells (alternatively put, equal to or less than about 5% of cells in the product are CD4+CD25− cells). For example, the product may comprise equal to or less than about 4% or equal to or less than about 3% or equal to or less than about 2% or equal to or less than about 1% CD4+CD25− cells. For example, the product may comprise from about 0.1% to about 5% or from about 0.5% to about 4% of CD4+CD25− cells.

There is further provided herein a pharmaceutical composition comprising the product obtained by and/or obtainable by the in vitro method described herein, including any embodiment thereof.

A pharmaceutical composition is a composition that comprises or consists of a therapeutically effective amount of a pharmaceutically active agent i.e. the cell (e.g. Treg). It preferably includes a pharmaceutically acceptable carrier, diluent or excipient (including combinations thereof). Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s) or solubilising agent(s).

By “pharmaceutically acceptable” it is meant that the formulation is sterile and pyrogen free. The carrier, diluent, and/or excipient must be “acceptable” in the sense of being compatible with the cell or vector and not deleterious to the recipients thereof. Typically, the carriers, diluents, and excipients will be saline or infusion media which will be sterile and pyrogen free, however, other acceptable carriers, diluents, and excipients may be used.

Examples of pharmaceutically acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

### Uses

The in vitro method described herein may be used to reduce the proportion or amount of one or more contaminating cells (e.g CD8+ T-cells and/or CD4+CD25− T-cells) in a population of Tregs and/or to inhibit the expansion of CD8+ T-cells and/or to inhibit the expansion of CD4+CD25− T-cells.

Thus, there is further provided herein a method for reducing the proportion or amount of one or more contaminating cells (e.g. CD8+ T-cells and/or CD4+CD25− T-cells) in a population of Tregs and/or a method of inhibiting the expansion of CD8+ T-cells and/or a method of inhibiting the expansion of CD4+CD25− T-cells, the method comprising steps (a), (b) and (c) of the in vitro method described herein. The method may also further comprise any of the other steps described herein and may thus be in accordance with any embodiment of the in vitro method described herein.

A reduction in the proportion or amount of one or more contaminating cells means that there is a decrease in the proportion or amount of contaminating cells in the final product of the method in comparison to either the starting material (population of Tregs used in step (a)) and/or in comparison to the product of a corresponding method in which step (a) is not performed and/or in which step (c) is performed in the absence of an mTOR inhibitor. The “corresponding method” is as described above. The proportion or amount of contaminating cells in a population of Tregs can be determined by FACS.

For example, the proportion or amount of one or more contaminating cells, particularly CD8+ T-cells, may be reduced by at least about 50% or at least about 55% or at least about 60% or at least about 65% or at least about 70% or at least about 75% as compared to the product of a corresponding method in which step (a) is not performed and/or step (c) is performed in the absence of an mTOR inhibitor or as compared to the starting material used in step (a). For example, the proportion or amount of one or more contaminating cells, particularly CD8+ T-cells, may be reduced by up to about 99% or up to about 95% or up to about 90% or up to about 85% or up to about 80% as compared to the product of a corresponding method in which step (a) is not performed and/or step (c) is performed in the absence of an mTOR inhibitor or as compared to the starting material used in step (a). For example, the proportion or amount of one or more contaminating cells, particularly CD8+ T-cells, may be reduced by about 50% to about 99% or from about 60% to about 95% or from about 65% to about 90% as compared to the product of a corresponding method in which step (a) is not performed and/or step (c) is performed in the absence of an mTOR inhibitor or as compared to the starting material used in step (a).

For example, the proportion of one or more contaminating cells, particularly CD8+ T-cells, may be reduced by at least about 0.1 percentage points or at least about 0.2 percentage points or at least about 0.3 percentage points or at least about 0.4 percentage points or at least about 0.5 percentage points or at least about 1 percentage point or at least about 2 percentage points or at least about 3 percentage points or at least about 4 percentage points or at least about 5 percentage points or at least about 6 percentage points or at least about 7 percentage points or at least about 8 percentage points or at least about 9 percentage points or at least about 10 percentage points relative to the starting material used in step (a). For example, the proportion of one or more contaminating cells, particularly CD8+ T-cells, may be reduced by up to about 20 percentage points or up to about 15 percentage points relative to the starting material used in step (a).

Inhibition of the expansion of CD8+ T-cells and/or CD4+CD25− T-cells means that CD8+ and/or CD4+CD25− T-cells may be prevented from proliferating or that proliferation is reduced and thus the total number or proportion of CD8+ and/or CD4+CD25− T-cells respectively in the final product of the method does not increase in comparison to either the starting material (population of Tregs used in step (a)) and/or in comparison to the product of a corresponding method in which step (a) is not performed and/or in which step (c) is performed in the absence of an mTOR inhibitor. Further, the proportion and/or amount of CD8+ and/or CD4+CD25− T-cells in the final product may be reduced as described above. The “corresponding method” is as described above. The total number and/or proportion of certain cells in a population of Tregs can be determined using an automated cell counter and FACS.

The in vitro method described herein may be used to increase the proportion or amount of Tregs and/or increase the proportion or amount of cells expressing FOXP3 and/or increase the proportion or amount of cells having a demethylated Treg-specific demethylated region (TSDR) in a population of Tregs and/or increase the proportion or amount of FOXP3+ Tregs and/or increase the proportion or amount of Helios+ Tregs and/or increase the proportion or amount of CD27+ Tregs.

Thus, there is further provided herein a method for increasing the proportion or amount of Tregs and/or increasing the proportion of cells expressing FOXP3 and/or increasing the proportion or amount of cells having a demethylated Treg-specific demethylated region (TSDR) in a population of Tregs and/or increasing the proportion or amount of FOXP3+ Tregs and/or increasing the proportion or amount of Helios+ Tregs and/or increasing the proportion or amount of CD27+ Tregs, the method comprising steps (a), (b) and (c) of the in vitro method described herein. The method may also further comprise any of the other steps described herein and may thus be in accordance with any embodiment of the in vitro method described herein.

An increase in the proportion or amount of Tregs and/or cells expressing FOXP3 and/or cells having a demethylated TSDR and/or FOXP3+ Tregs and/or Helios+ Tregs and/or CD27+ Tregs means that there is an increase in the proportion or amount of Tregs and/or cells expressing FOXP3 and/or cells having a demethylated TSDR and/or FOXP3+ Tregs and/or Helios+ Tregs and/or CD27+ Tregs respectively in the final product of the method in comparison to either the starting material (population of Tregs used in step (a)) and/or in comparison to the product of a corresponding method in which step (a) is not performed and/or in which step (c) is performed in the absence of an mTOR inhibitor. The “corresponding method” is as described above. The proportion or amount of Tregs and/or cells expressing FOXP3 and/or FOXP3+ Tregs and/or Helios+ Tregs and/or CD27+ Tregs and proportion or amount of cells with a demethylated TSDR in a population of Tregs can be determined by FACS or MS-qPCR as described above.

For example, the proportion or amount of CD4+CD25+FOXP3+ T-cells may increase by at least about 3% or at least about 5% or at least about 10% or at least about 15% or at least about 20% as compared to the product of a corresponding method in which step (a) is not performed and/or step (c) is performed in the absence of an mTOR inhibitor or normalized to the starting material used in step (a). For example, the proportion or amount of CD4+CD25+FOXP3+ T-cells may increase by up to about 150% or up to about 100% or up to about 90% or up to about 80% as compared to the product of a corresponding method in which step (a) is not performed and/or step (c) is performed in the absence of an mTOR inhibitor or normalized to the starting material used in step (a). For example, the proportion or amount of CD4+CD25+FOXP3+ T-cells may increase by from about 3% to about 150% or from about 5% to about 100% or from about 10% to about 80% as compared to the product of a corresponding method in which step (a) is not performed and/or step (c) is performed in the absence of an mTOR inhibitor or normalized to the starting material used in step (a).

For example, the proportion of Tregs may increase by at least about 3 percentage points or at least about 5 percentage points or at least about 10 percentage points or at least about 15 percentage points or at least about 20 percentage points or at least about 25 percentage points or at least about 30 percentage points relative to the starting material used in step (a). For example, the proportion of Tregs may increase by up to about 75 percentage points or up to about 70 percentage points or up to about 65 percentage points or up to about 60 percentage points or up to about 55 percentage points or up to about 50 percentage points relative to the starting material used in step (a). Tregs may have any Treg phenotype as described herein, for example CD4+CD25+FOXP3+.

For example, the proportion or amount of Helios+ Tregs may increase by at least about 3% or at least about 5% or at least about 10% or at least about 15% or at least about 20% as compared to the product of a corresponding method in which step (a) is not performed and/or step (c) is performed in the absence of an mTOR inhibitor or normalized to the starting material used in step (a). For example, the proportion or amount of Helios+ Tregs may increase by up to about 150% or up to about 100% or up to about 90% or up to about 80% as compared to the product of a corresponding method in which step (a) is not performed and/or step (c) is performed in the absence of an mTOR inhibitor or normalized to the starting material used in step (a). For example, the proportion or amount of Helios+ Tregs may increase by from about 3% to about 150% or from about 5% to about 100% or from about 10% to about 80% as compared to the product of a corresponding method in which step (a) is not performed and/or step (c) is performed in the absence of an mTOR inhibitor or normalized to the starting material used in step (a). The Helios+ Tregs may have any Treg phenotype as described herein, for example CD4+CD25+FOXP3+.

For example, the proportion of Helios+ Tregs may increase by at least about 3 percentage points or at least about 5 percentage points or at least about 10 percentage points or at least about 15 percentage points or at least about 20 percentage points or at least about 25 percentage points or at least about 30 percentage points relative to the starting material used in step (a). For example, the proportion of Helios+ Tregs may increase by up to about 75 percentage points or up to about 70 percentage points or up to about 65 percentage points or up to about 60 percentage points or up to about 55 percentage points or up to about 50 percentage points relative to the starting material used in step (a). Helios+ Tregs may have any Treg phenotype as described herein, for example CD4+CD25+FOXP3+.

For example, the proportion or amount of CD27+ Tregs may increase by at least about 3% or at least about 5% or at least about 10% or at least about 15% or at least about 20% as compared to the product of a corresponding method in which step (a) is not performed and/or step (c) is performed in the absence of an mTOR inhibitor or normalized to the starting material used in step (a). For example, the proportion or amount of CD27+ Tregs may increase by up to about 150% or up to about 100% or up to about 90% or up to about 80% as compared to the product of a corresponding method in which step (a) is not performed and/or step (c) is performed in the absence of an mTOR inhibitor or normalized to the starting material used in step (a). For example, the proportion or amount of CD27+ Tregs may increase by from about 3% to about 150% or from about 5% to about 100% or from about 10% to about 80% as compared to the product of a corresponding method in which step (a) is not performed and/or step (c) is performed in the absence of an mTOR inhibitor or normalized to the starting material used in step (a). The CD27+ Tregs may have any Treg phenotype as described herein, for example CD4+CD25+FOXP3+.

For example, the proportion of CD27+ Tregs may increase by at least about 3 percentage points or at least about 5 percentage points or at least about 10 percentage points or at least about 15 percentage points or at least about 20 percentage points or at least about 25 percentage points or at least about 30 percentage points relative to the starting material used in step (a). For example, the proportion of CD27+ Tregs may increase by up to about 75 percentage points or up to about 70 percentage points or up to about 65 percentage points or up to about 60 percentage points or up to about 55 percentage points or up to about 50 percentage points relative to the starting material used in step (a). CD27+ Tregs may have any Treg phenotype as described herein, for example CD4+CD25+FOXP3+.

For example, the proportion or amount of TSDR demethylation in the product may increase by at least about 3% or at least about 5% or at least about 10% or at least about 15% or at least about 20% as compared to the product of a corresponding method in which step (a) is not performed and/or step (c) is performed in the absence of an mTOR inhibitor or normalized to the starting material used in step (a). For example, the proportion or amount of TSDR demethylation may increase by up to about 150% or up to about 100% or up to about 90% or up to about 80% as compared to the product of a corresponding method in which step (a) is not performed and/or step (c) is performed in the absence of an mTOR inhibitor or normalized to the starting material used in step (a). For example, the proportion or amount of TSDR demethylation may increase by from about 3% to about 150% or from about 5% to about 100% or from about 10% to about 80% as compared to the product of a corresponding method in which step (a) is not performed and/or step (c) is performed in the absence of an mTOR inhibitor or normalized to the starting material used in step (a).

For example, TSDR demethylation may increase by at least about 3 percentage points or at least about 5 percentage points or at least about 10 percentage points or at least about 15 percentage points or at least about 20 percentage points or at least about 25 percentage points or at least about 30 percentage points relative to the starting material used in step (a). For example, TSDR demethylation may increase by up to about 75 percentage points or up to about 70 percentage points or up to about 65 percentage points or up to about 60 percentage points or up to about 55 percentage points or up to about 50 percentage points relative to the starting material used in step (a).

The in vitro method described herein may be used to increase the proportion of CD45RA+ Tregs in a population of Tregs. Thus, there is further provided herein a method for increasing the proportion of CD45RA+ Tregs in a population of Tregs, the method comprising steps (a), (b) and (c) of the in vitro method described herein. The method may also further comprise any of the other steps described herein and may thus be in accordance with any embodiment of the in vitro method described herein. An increase in the proportion of CD45RA+ Tregs means that there is an increase in the proportion of CD45RA+ Tregs in the final product of the method in comparison to the product of a corresponding method in which step (a) is not performed and/or in which step (c) is performed in the absence of an mTOR inhibitor. The “corresponding method” is as described above. The proportion of CD45RA+ Tregs in a population of Tregs can be determined by FACS.

For example, the proportion of CD45RA+ Tregs may increase by at least about 3% or at least about 5% or at least about 10% or at least about 15% or at least about 20% as compared to the product of a corresponding method in which step (a) is not performed and/or step (c) is performed in the absence of an mTOR inhibitor. For example, the proportion of CD45RA+ Tregs may increase by up to about 150% or up to about 100% or up to about 90% or up to about 80% as compared to the product of a corresponding method in which step (a) is not performed and/or step (c) is performed in the absence of an mTOR inhibitor. For example, the proportion of CD45RA+ Tregs may increase by from about 3% to about 150% or from about 5% to about 100% or from about 10% to about 80% as compared to the product of a corresponding method in which step (a) is not performed and/or step (c) is performed in the absence of an mTOR inhibitor.

For example, the proportion of CD45RA+ Tregs may increase by at least about 2 percentage points or at least about 3 percentage points or at least about 4 percentage points or at least about 5 percentage points or at least about 10 percentage points or at least about 15 percentage points as compared to the product of a corresponding method in which step (a) is not performed and/or step (c) is performed in the absence of an mTOR inhibitor. For example, the proportion of Tregs may increase by up to about 75 percentage points or up to about 70 percentage points or up to about 65 percentage points or up to about 60 percentage points or up to about 55 percentage points or up to about 50 percentage points or up to about 45 percentage points or up to about 40 percentage points as compared to the product of a corresponding method in which step (a) is not performed and/or step (c) is performed in the absence of an mTOR inhibitor. Tregs may have any Treg phenotype as described herein, for example CD4+CD25+FOXP3+ or CD4+CD25− CD127−/low.

The in vitro method described herein may be used to increase the suppressive function of a population of Tregs.

Thus, there is further provided herein a method for increasing the suppressive function of a population of Tregs, the method comprising steps (a), (b) and (c) of the in vitro method described herein. The method may also further comprise any of the other steps described herein and may thus be in accordance with any embodiment of the in vitro method described herein.

An increase in the suppressive function of a population of Tregs means that there is an increase in the suppressive function of a population of Tregs in comparison to either the starting material (population of Tregs used in step (a)) and/or in comparison to the product of a corresponding method in which step (a) is not performed and/or in which step (c) is performed in the absence of an mTOR inhibitor. The “corresponding method” is as described above. An increase in suppressive function can be an increase of at least 10, 20, 30, 40, 50, 60, 70, 80 or 90% e.g., in comparison to either the suppressive function of the starting material (Treg population used in step (a)) and/or in comparison to the product of a corresponding method in which step (a) is not performed and/or in which step (c) is performed in the absence of an mTOR inhibitor,

The suppressive function of a population of Tregs can be determined by any method described herein, for example by measuring suppression of Teff proliferation by Tregs (e.g. at one particular Teff:Treg ratio) and/or by measuring cytokine expression of the product.

For example, cytokine expression (e.g. IL-17 or IFNγ) may decrease by at least about 10% or at least about 20% or at least about 30% or at least about 40% or at least about 50% or at least about 60% or at least about 70% as compared to cytokine expression of a product of a corresponding method in which step (a) is not performed and/or step (c) is performed in the absence of an mTOR inhibitor or as compared to the starting material used in step (a). For example, cytokine expression may decrease by up to 99% or up to 95% or up to about 90% as compared to cytokine expression of a product of a corresponding method in which step (a) is not performed and/or step (c) is performed in the absence of an mTOR inhibitor or as compared to the starting material used in step (a).

For example, suppression of Teff proliferation may decrease by at least about 10% or at least about 20% or at least about 30% or at least about 40% or at least about 50% or at least about 60% or at least about 70% as compared to suppression of Teff proliferation by a product of a corresponding method in which step (a) is not performed and/or step (c) is performed in the absence of an mTOR inhibitor or as compared to the starting material used in step (a). For example, suppression of Teff proliferation may decrease by up to 99% or up to 95% or up to about 90% as compared to suppression of Teff proliferation by a product of a corresponding method in which step (a) is not performed and/or step (c) is performed in the absence of an mTOR inhibitor or as compared to the starting material used in step (a).

The in vitro method described herein may be used to increase the expansion of a population of Tregs and/or to increase the expansion of Tregs in a population of Tregs.

Thus, there is further provided herein a method for increasing the expansion of a population of Tregs and/or a method for increasing the expansion of Tregs in a population of Tregs, the method comprising steps (a), (b) and (c) of the in vitro method described herein. The method may also further comprise any of the other steps described herein and may thus be in accordance with any embodiment of the in vitro method described herein.

An increase in the expansion of a population of Tregs or Tregs in a population of Tregs means that there is an increase in the fold expansion of the population of Tregs or Tregs in the population of Tregs respectively obtained by the method of the invention in comparison to the fold expansion obtained using a corresponding method in which step (a) is not performed and/or in which step (c) is performed in the absence of an mTOR inhibitor. The “corresponding method” is and method for measuring fold expansion is described above.

For example, the fold expansion of a population of Tregs or Tregs in a population of Tregs may increase by at least about 5% or at least about 10% or at least about 15% or at least about 20% or at least about 25% or at least about 30% normalized to the fold expansion of a corresponding method in which step (a) is not performed and/or step (c) is performed in the absence of an mTOR inhibitor. For example, the fold expansion may increase by up to about 100% or up to about 90% or up to about 80% or up to about 70% or up to about 60% or up to about 50% normalized to the fold expansion of a corresponding method in which step (a) is not performed and/or step (c) is performed in the absence of an mTOR inhibitor.

Where the in vitro method comprises a step of introducing a heterologous nucleic acid into the cells, the method may increase the expression of the heterologous nucleic acid and/or increase the transduction efficiency of the heterologous nucleic acid and/or decrease the vector copy number (VCN) of the heterologous nucleic acid. Thus, there is further provided herein a method for increasing the expression of a heterologous nucleic acid and/or increasing the transduction efficiency of the heterologous nucleic acid and/or decreasing the VCN of the heterologous nucleic acid, the method comprising steps (a), (b) and (c) of the in vitro method described herein. The method may also further comprise any of the other steps described herein and may thus be in accordance with any embodiment of the in vitro method described herein.

An increase in the expression of the heterologous nucleic acid means that there is an increase in the expression of the heterologous nucleic acid in the population of Tregs in comparison to the product of a corresponding method in which step (a) is not performed and/or in which step (c) is performed in the absence of an mTOR inhibitor. The “corresponding method” is and method for measuring expression of the heterologous nucleic (particularly one or more transgenes encoded by the heterologous nucleic acid) is as described above.

For example, expression of the heterologous nucleic acid (or one or more transgenes encoded by the heterologous nucleic acid) may increase by at least about 5% or at least about 10% or at least about 15% or at least about 20% as compared to the product of a corresponding method in which step (a) is not performed and/or step (c) is performed in the absence of an mTOR inhibitor, or as compared to the starting material used in step (a). For example, expression of the heterologous nucleic acid (or one or more transgenes encoded by the heterologous nucleic acid) may increase by up to about 100% or up to about 90% or up to about 80% or up to about 70% or up to about 60% or up to about 50% as compared to the product of a corresponding method in which step (a) is not performed and/or step (c) is performed in the absence of an mTOR inhibitor, or compared to the starting material used in step (a).

An increase in the transduction efficiency of the heterologous nucleic acid means that there is an increase in the transduction efficiency of the heterologous nucleic acid in the population of Tregs in comparison to the product of a corresponding method in which step (a) is not performed and/or in which step (c) is performed in the absence of an mTOR inhibitor. The “corresponding method” is as described above. Transduction efficiency may be determined by detecting the presence of the heterologous nucleic acid or the presence of a heterologous protein encoded by the heterologous nucleic acid in the cells.

For example, the transduction efficiency of the heterologous nucleic acid (or one or more transgenes encoded by the heterologous nucleic acid) may increase by at least about 5% or at least about 10% or at least about 15% or at least about 20% as compared to the product of a corresponding method in which step (a) is not performed and/or step (c) is performed in the absence of an mTOR inhibitor. For example, the transduction efficiency of the heterologous nucleic acid (or one or more transgenes encoded by the heterologous nucleic acid) may increase by up to about 100% or up to about 90% or up to about 80% or up to about 70% or up to about 60% or up to about 50% as compared to the product of a corresponding method in which step (a) is not performed and/or step (c) is performed in the absence of an mTOR inhibitor.

For example, the transduction efficiency of the heterologous nucleic acid (or one or more transgenes encoded by the heterologous nucleic acid) may increase by at least about 3 percentage points or at least about 5 percentage points or at least about 10 percentage points or at least about 15 percentage points as compared to the product of a corresponding method in which step (a) is not performed and/or step (c) is performed in the absence of an mTOR inhibitor. For example, the transduction efficiency of the heterologous nucleic acid (or one or more transgenes encoded by the heterologous nucleic acid) may increase by up to about 40 percentage points or up to about 35 percentage points or up to about 30 percentage points or up to about 25 percentage point or up to about 20 percentage points as compared to the product of a corresponding method in which step (a) is not performed and/or step (c) is performed in the absence of an mTOR inhibitor.

A decrease in the VCN of the heterologous nucleic acid means that there is an decrease in the VCN of the heterologous nucleic acid in the population of Tregs in comparison to the product of a corresponding method in which step (a) is not performed and/or in which step (c) is performed in the absence of an mTOR inhibitor. The “corresponding method” is as described above. VCN may be determined using digital/quantitative PCR.

For example, the VCN of the heterologous nucleic acid (or one or more transgenes encoded by the heterologous nucleic acid) may decrease by at least about 0.5 copies per transduced cell or at least about 1 copy per transduced cell as compared to the product of a corresponding method in which step (a) is not performed and/or step (c) is performed in the absence of an mTOR inhibitor. For example, the VCN of the heterologous nucleic acid (or one or more transgenes encoded by the heterologous nucleic acid) may decrease by up to about 5, 4, 3 or 2 copies per transduced cell as compared to the product of a corresponding method in which step (a) is not performed and/or step (c) is performed in the absence of an mTOR inhibitor.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist one of ordinary skill in the art in carrying out the invention and are not intended in any way to limit the scope of the invention.

### EXAMPLES

### Example 1—Composition of the Cell Product

### Materials and Methods

### Starting Material:

Leukopaks were supplied by BioIVT. PBMCs were isolated using a negative selection kit (StemCell Technologies). Briefly, unwanted fractions are labelled and targeted for removal with antibody complexes and magnetic beads and subsequently separated using a magnet.

Leukopaks were used to derive Treg populations. Leukopak-derived PBMCs were subjected to CD25 positive selection followed by a CD4 enrichment via negative selection using Human CD25 Positive Selection Cocktail and Human CD4+ T Cell Enrichment Cocktail (Stemcell Technologies), respectively. Cells of interest were separated using a magnet. CD127high Depletion Cocktail (Stemcell Technologies) was used to further separate the CD4+CD25+CD127−/low cell populations and cells of interest were separated using a magnet. Cell fractions were stained with flow cytometry antibodies anti-CD4 VioBlue (M-T466 Miltenyi), anti-CD25 PE (3G10, Miltenyi), anti-CD45RA FITC (T6D11, Miltenyi) and anti-CD127 APC (MB15-18C, Miltenyi) before FACS sorting. Where indicated, CD4+CD25+CD127−/low (Bulk Tregs) were used.

In order to obtain CD8+ T-cells for spiking, a small fraction of leukopak-derived PBMCs was kept and subjected to negative selection using human CD8+ T Cell Isolation Cocktail (Stemcell Technologies). Cells of interest were separated using a magnet. A small cell fraction was stained with flow cytometry antibodies anti-CD3 APC (UCHT1, Biolegend) and anti-CD8a PE (RPA-T8 Biolegend) in order to check for CD8 purity. Isolated CD8+ cells were rested in X-VIVO 15 media (Lonza) supplemented with 5% Human AB serum (heat-inactivated; Merck) until sorted Tregs were ready to be spiked.

Leukopaks were obtained from healthy volunteers or liver transplant patients. Where indicated, bulk Tregs obtained from healthy volunteers were spiked with 5% or 10% CD8+ T-cells, which were also obtained from the same subject.

### Lentivirus

A lentiviral vector was used to transduce the cells. The vector encodes an RQR8 safety switch, a FOXP3 polypeptide and an HLA-A2-specific CAR.

### Expansion Protocol:

### “1+24” or “1+48” Conditions:

The Tregs were rested in Treg culture media (X-VIVO 15 media (Lonza) supplemented with 5% Human AB serum (heat-inactivated; Merck)) containing 100 nM of rapamycin for 1 hour before activation. The Tregs were then activated by adding CD3 and CD28 beads and cultured for 24 or 48 hours (1+24 and 1+48 conditions respectively). At 24 or 48 hours, the cells were washed with fresh Treg culture media and centrifuged. The supernatant was removed and the cell pellet was resuspended in fresh Treg culture media. The expansion was then continued in the fresh Treg culture media (X-VIVO 15 media (Lonza) supplemented with 5% Human AB serum (heat-inactivated; Merck)) further containing IL-2 (Proleukin, Clinigen Healthcare) and lentiviral supernatant but no rapamycin. Cells were re-fed every 2 to 3 days with Treg culture media supplemented with IL-2. Cells were harvested by removing the CD3 and CD28 beads 14 days after activation.

### “1 hr Pre-Treatment”:

The Tregs were rested in Treg culture media (X-VIVO 15 media (Lonza) supplemented with 5% Human AB serum (heat-inactivated; Merck)) containing 100 nM of rapamycin for 1 hour before activation. At 1 hour, the cells were washed with fresh Treg culture media and centrifuged. The supernatant was removed and the cell pellet was resuspended in fresh Treg culture media (containing no rapamycin). The Tregs were then activated by adding CD3 and CD28 beads to the fresh Treg culture media and cultured. At 48 hours, the culture media was removed and the expansion was continued in fresh Treg culture media (X-VIVO 15 media (Lonza) supplemented with 5% Human AB serum (heat-inactivated; Merck)) further containing IL-2 (Proleukin, Clinigen Healthcare) and lentiviral supernatant (but no rapamycin). Cells were re-fed every 2 to 3 days with Treg culture media supplemented with IL-2. Cells were harvested by removing the CD3 and CD28 beads 14 days after activation.

### “24 hr or 48 hr Culture”:

Tregs were activated by adding CD3 and CD28 beads to Treg culture media (X-VIVO 15 media (Lonza) supplemented with 5% Human AB serum (heat-inactivated; Merck)) containing 100 nM of rapamycin. At 24 or 48 hours (24 hr or 48 hr conditions respectively), the cells were washed with fresh Treg culture media (containing no rapamycin) and centrifuged. The supernatant was removed and the cell pellet was resuspended in fresh Treg culture media. The expansion was then continued in the fresh Treg culture media (X-VIVO 15 media (Lonza) supplemented with 5% Human AB serum (heat-inactivated; Merck)) further containing IL-2 (Proleukin, Clinigen Healthcare) and lentiviral supernatant (but no rapamycin). Cells were re-fed every 2 to 3 days with Treg culture media supplemented with IL-2. Cells were harvested by removing the CD3 and CD28 beads 14 days after activation.

### “No Rapa” (Control):

The Tregs were rested in Treg culture media (X-VIVO 15 media (Lonza) supplemented with 5% Human AB serum (heat-inactivated; Merck)) for 1 hour before activation. The Tregs were then activated by adding CD3 and CD28 beads and cultured for 24 or 48 hours. If the condition is a control for the 24 hr or 1+24 hr condition, the Tregs are cultured for 24 hours. If the condition is a control for the 1 hr pre-treatment condition, 48 hr or 1+48 hr condition, the Tregs are cultured for 48 hours. At 24 or 48 hours, the cells were washed with fresh Treg culture media (containing no rapamycin) and centrifuged. The supernatant was removed and the cell pellet was resuspended in fresh Treg culture media. The expansion was then continued in the fresh Treg culture media (X-VIVO 15 media (Lonza) supplemented with 5% Human AB serum (heat-inactivated; Merck)) further containing IL-2 (Proleukin, Clinigen Healthcare) and lentiviral supernatant (but no rapamycin). Cells were re-fed every 2 to 3 days with Treg culture media supplemented with IL-2. Cells were harvested by removing the CD3 and CD28 beads 14 days after activation.

### Analysis of Cell Population:

The % of cells expressing FOXP3 and % of CD8+ cells present in the product harvested at day 14 was determined by FACS.

Tregs were first surface stained with Dextramer PE (CINGVCWTV-NS3 Hepatitis-HLA-A*0202, Immudex) in FACS staining buffer), the LIVE/DEAD™ Fixable Near-IR—Dead Cell Stain (Thermofisher) in PBS and then with anti-CD4 V450 (SK3, BD Bioscience), anti-CD8 BV605 (RPA-T8, Biolegend), anti-RQR8 FITC (QBEND, Invitrogen), anti-CD19 PE-Cy7 (HIB19, Biolegend) and anti-CD56 APC-R700 (NCAM16.2, BD Bioscience) in FACS staining buffer. For intracellular staining of FOXP3, cells may be fixed and permeabilized and stained with the anti-FoxP3 AF647 (259D, Biolegend). Cells were washed before acquisition on the BD FACsLyric™ flow cytometer. FoxP3 expression was analysed using FlowJo software. The following gating strategy was used: Lymphocytes>single cells>viable cells>CD19−CD56−>CD4+>CD4+CD25+>FoxP3+.

### Results

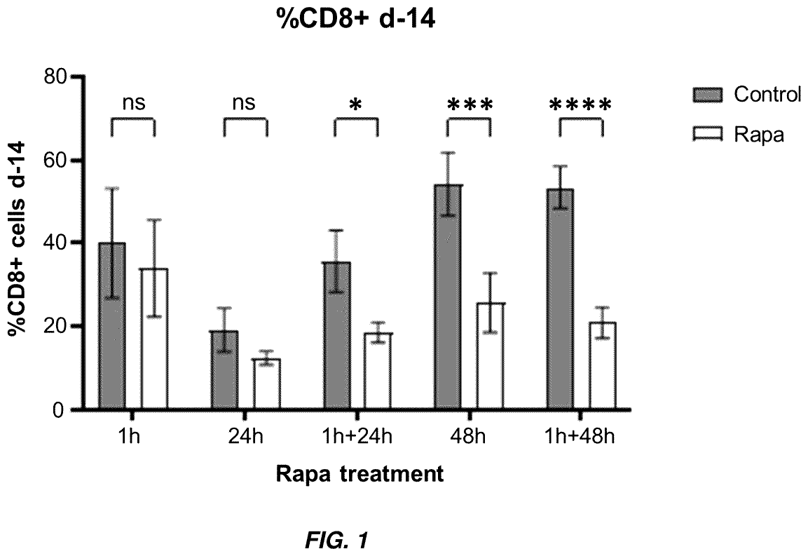

FIG. 1 shows the % of CD8+ cells at day 14 for products expanded from bulk Tregs from healthy donors spiked with 5% CD8+ cells. The bulk Treg starting material from each healthy donor was split in half and then expanded with either no rapa (control) or with one of the rapa conditions (1h, 24 h, 1 h+24 h, 48h or 1 h+48h). The bar shows the average % of CD8+ cells across all donors tested. It can be seen that the 1 h and 24h conditions did not significantly reduce % of CD8+ cells in the expansion product compared to no rapa (control) conditions [ns]. The 1+24, 48 and 1+48 conditions all significantly decreased % of CD8+ cells in the expansion product compared to no rapa (control) conditions (*, *** and **** respectively). The 1+24h condition resulted in a greater improvement in % of CD8+ cells compared to 24h condition. The 1+48h condition resulted in a higher level of significance (****) compared to the 48h condition (***), thus demonstrating a greater improvement. Both the 1+24 h and 1+48h conditions resulted in a greater improvement in % of CD8+ cells compared to 1h condition.

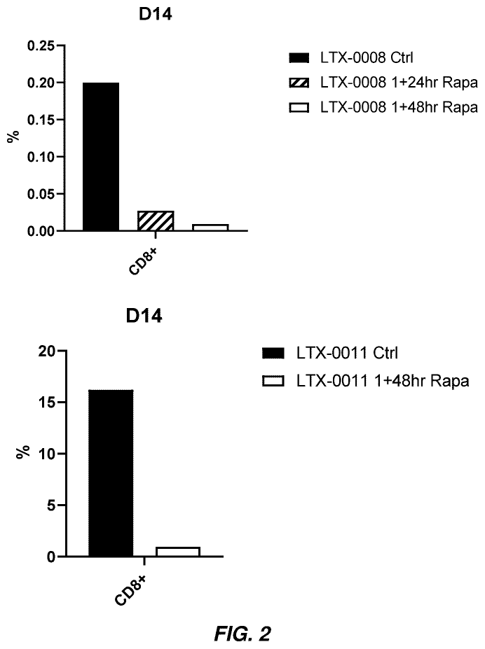

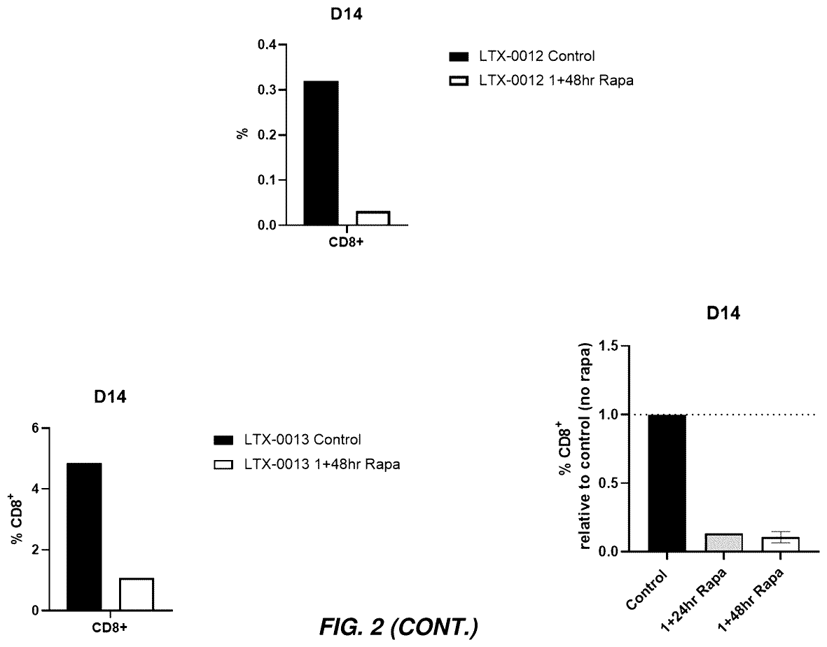

FIG. 2 shows the % of CD8+ cells at day 14 for products expanded from bulk Tregs from patient material (4 different patient samples—0008, 0011, 0012 and 0013). The average % of CD8+ cells at day 14 across all patient donors normalised to control is also shown. The material was split and expanded by under different conditions. The 1+24 h and 1+48h conditions both resulted in fewer CD8+ cells in the expansion product compared to no rapa (control) conditions.

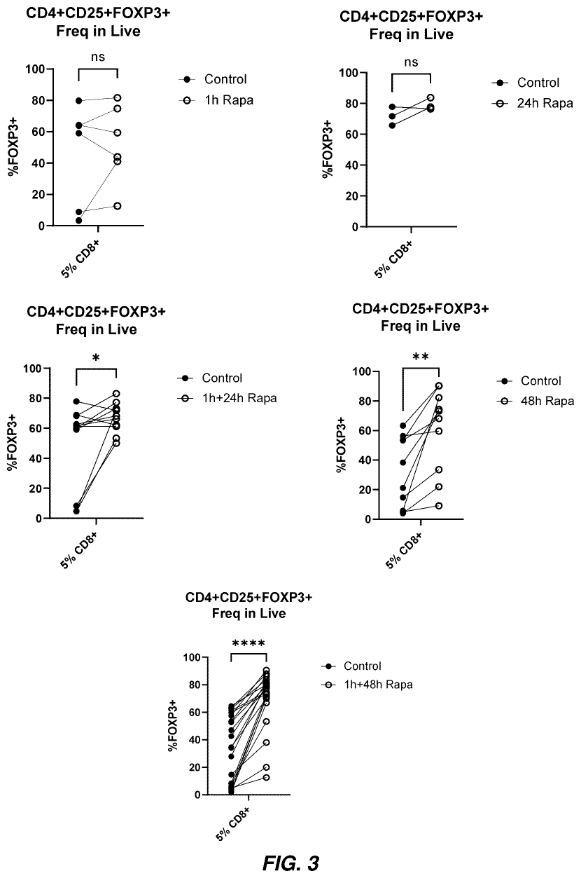

FIG. 3 shows the % of cells expressing FOXP3 at day 14 for products expanded from bulk Tregs from healthy donors spiked with 5% CD8+ cells. The bulk Treg starting material from each healthy donor was split in half and then expanded with either no rapa (control) or with one of the rapa conditions (1 h, 24 h, 1h+24 h, 48h or 1h+48h). It can be seen that the 1+24 h, 48h and 1+48h conditions significantly increased the % of FOXP3+ cells in the expansion product, however 1+48h conditions reached a higher level of significance (***) than the 48h conditions (**) and thus showed a greater improvement. The 1 h and 24h conditions did not significantly improve the % of FOXP3 cells in the expansion product.

Table 1 shows the % of FOXP3+ cells in the expansion product obtained from patient donor material using different conditions. It can be seen that the 1+24 h and 1+48h conditions both provide an improved product compared to no rapa (control) conditions.

TABLE 1

| Donor | Conditions | % CD4+CD25+FOXP3+ cells |
|---|---|---|
| LTX-008 | No rapa (control) | 80.8 |
| | 1 + 24 h | 91.2 |
| | 1 + 48 h | 91.4 |
| LTX-0011 | No rapa (control) | 57.3 |
| | 1 + 48 h | 92.6 |
| LTX-0012 | No rapa (control) | 92.8 |
| | 1 + 48 h | 98.2 |
| LTX-0013 | No rapa (control) | 41.0 |
| | 1 + 48 h | 89.9 |

### Example 2—Total Cell Expansion

### Materials and Methods

The same starting materials and expansion protocols as Example 1 were used.

Fold expansion at day 14 was calculated by dividing the number of Tregs collected on day 14 by the number seeded on day 0.

### Results

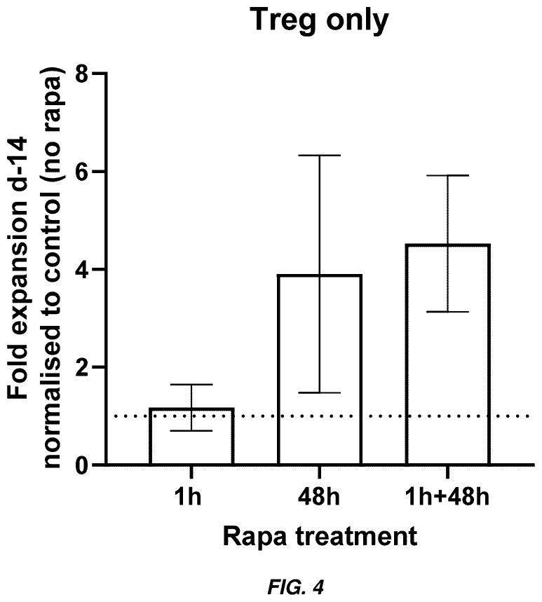

FIG. 4 shows fold expansion of Treg cells at day 14 for products expanded from bulk Tregs from healthy donors spiked with 5% CD8+ cells. The bulk Treg starting material from each healthy donor was split in half and then expanded with either no rapa (control) or with one of the rapa conditions. The fold expansion for the rapa condition was normalised to the no rapa (control) by dividing the fold expansion for a particular rapa condition by fold expansion for the no rapa (control) condition. It can be seen that the 1+48h conditions provides a markedly improved fold expansion compared to both 1 h and 48h conditions.

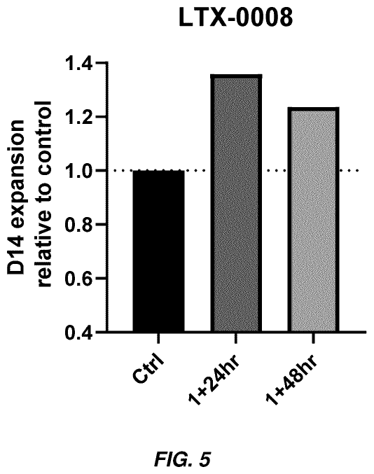

FIG. 5 shows fold expansion of all cells at day 14 for products expanded from bulk Tregs obtained from patient material (LTX-0008). It can be seen that 1+24 h and 1+48h conditions both provide improved fold expansion compared to no rapa (control) conditions.

### Example 3—IL-17 and IFNγ Production

### Materials and Methods

The same starting materials and expansion protocols as Example 1 were used.

IL-17 and IFNγ production was determined by cytometric bead array (CBA) assay on supernatants that were collected from day 14 Tregs that were stimulated with CD3/CD28 dynabeads (Gibco, final dilution 1:40 in X-VIVO 15 media (Lonza) supplemented with 5% Human AB serum (heat-inactivated; Merck)) for 24 hours. Briefly, frozen supernatants were thawed, exposed to the LEGENDplex™ Hu Th Cytokine Panel (12-plex) w/FP V02 (BioLegend) using fluorescence-encoded beads as per manufacturer's instructions and acquired on Attune™ NxT flow cytometer. Cytokine expression was analysed using the LEGENDplex™ Data Analysis Software.

### Results

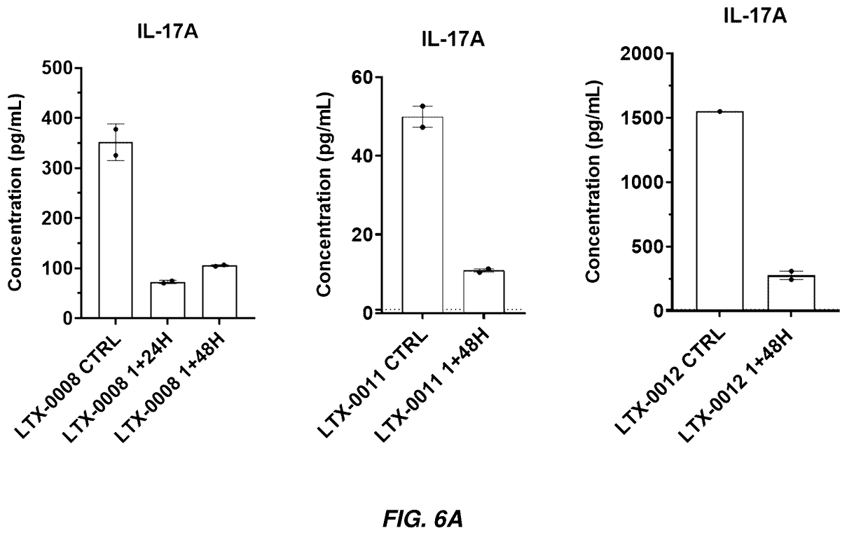

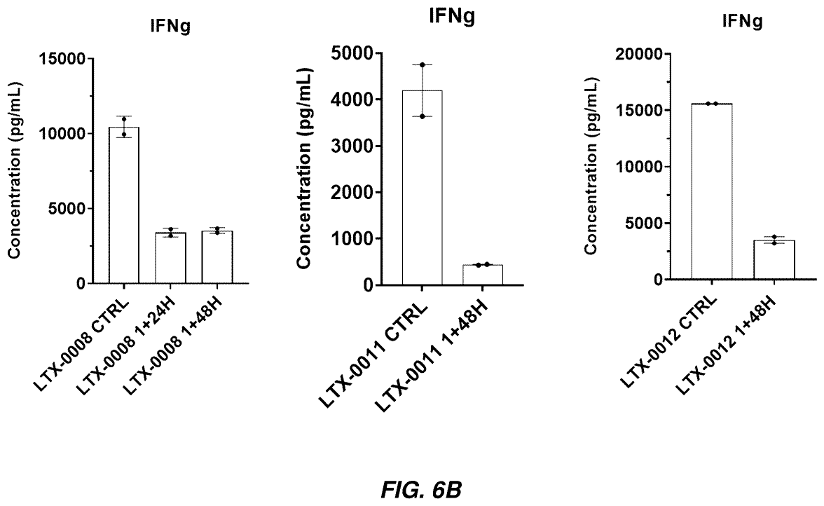

FIG. 6A shows the concentration of IL-17 and FIG. 6B shows the concentration of IFNγ produced by expansion products obtained from bulk Tregs obtained from patient materials. It can be seen that 1+24 h and 1+48h conditions both provide a reduced concentration of the pro-inflammatory cytokines IL-17 and IFNγ compared to no rapa (control) conditions.

### Example 4—Suppression Assay

### Materials and Methods

The same starting materials and expansion protocols as Example 1 were used.

Tregs were stained with CellTrace Yellow (Invitrogen) and added to a U-bottom 96 well plate with CellTrace Violet (Invitrogen) stained T effector cells at a range of ratios. Dynabeads were added at a ratio of 1:5 Bead:T effector cells, and the cells were incubated for 5 days at 37° C. in 5% CO₂ incubator. Cells were collected and incubated with Live/Dead NIR (Invitrogen) and Human trustain FcX (Biolegend) in PBS. Cells were then stained with anti-CD4 BV510 (A161A1; BioLegend) and anti-CD34 FITC (QBEND10; Invitrogen) in FACS staining buffer. Cells were washed before acquisition on the Attune™ NxT flow cytometer. Proliferation of the T effector cells was detected by dilution of the CellTrace Violet stain and analysed using FlowJo software. The % suppression of T effector proliferation was calculated as follows:

$$\% Suppression = \frac{( \% CTV diluted {Teff}_{0} - \% CTV diluted {Teff}_{i} )}{\% CTV diluted {Teff}_{0}} \times 100$$

i=various Treg:Tef f ratios
0=0:1 Treg:Tef f ratio

### Results

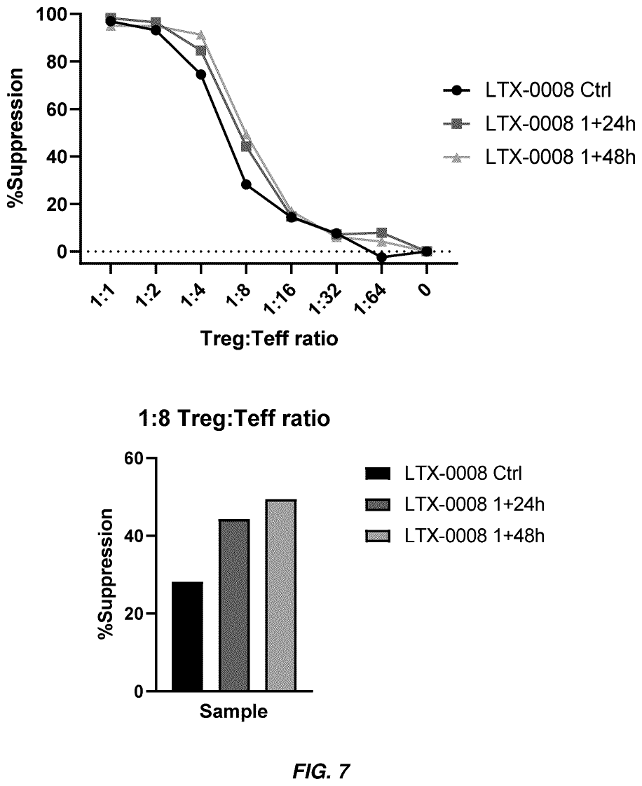

FIG. 7 shows the suppression of T effector cell (Teff) proliferation by Treg cells, across a range of Treg:Teff ratios. It can be seen that at intermediate Treg:Teff ratios 1+24 h and 1+48h rapamycin treated Treg cells show greater suppression of Teff proliferation than the untreated (Ctrl) cells.

### Example 5—Transgene Expression

### Materials and Methods

The same starting materials and expansion protocols as Example 1 were used.

Briefly, frozen Treg cells were thawed, counted and resuspended at 2×10⁶ Cells/mL in Treg culture media (X-VIVO 15 media (Lonza) supplemented with 5% Human AB serum (heat-inactivated; Merck)). 1×10⁵ Treg cells were transferred to each desired well of a U-bottom 96-well plate. A two-fold serial dilution of rituximab (Midwinter) in reconstituted rabbit complement (bRc) (Bio-Rad) was performed to achieve the following rituximab concentrations: 200 μg/mL, 100 μg/mL, 50 μg/mL, 25 μg/mL, 12.5 μg/mL. Each concentration of rituximab was added to the plated Treg cells in duplicate and incubated for 4 hours at 37° C. in 5% CO₂ incubator. Following the incubation, the plate was centrifuged and washed with FACS buffer. The cells were stained with the following antibodies: Dextramer APC (CINGVCWTV, Immudex), Live/Dead Near IR (Invitrogen), CD4 BV510 (A161A1, Biolegend), Qbend PE (Qbend10, Thermo Fisher) and Fc block (Biolegend) before FACS analysis.

Depletion of transgene expressing cells was determined by observing the % killing obtained with the various concentrations of rituximab. The % killing was calculated as follows:

$$\% Killing = \frac{( \% Dextramer without rM ab - \% Dextramer rM {ab}_{i} )}{\% Dextramer without rM ab} \times 100$$

rMab=Rituximab
i=various concentrations of rMab

### Results

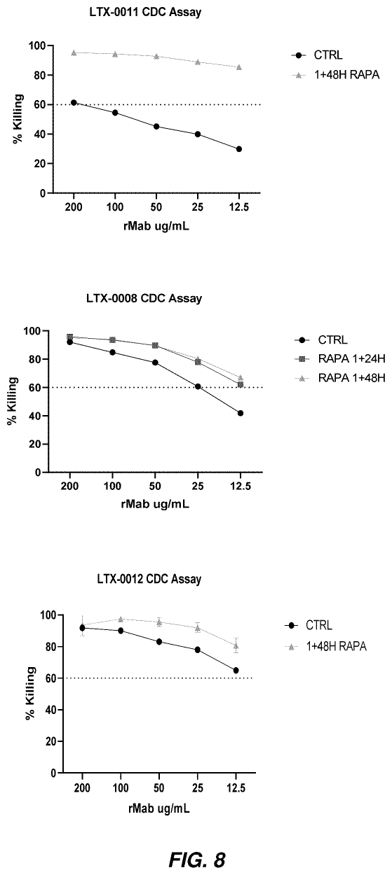

FIG. 8 shows % killing when using different concentrations of rituximab and is normalized to % dextramer expression. It can be seen that the expansion product obtained from patient material under 1+24 h and 1+48h conditions provides improved depletion compared to the expansion product obtained from the same patient material but under no rapa (control) conditions. The transduction efficiency for the expansion product obtained under all of these conditions was very similar (see Table 2 below). Therefore, the improvement in % killing is a result of improved transgene expression.

TABLE 2

| Donor | Conditions | % Dextramer+/QBEND+ |
|---|---|---|
| LTX-008 | Control | 36 |
| | 1 + 24 | 30 |
| | 1 + 48 | 37.8 |
| LTX-0011 | Control | 20.5 |
| | 1 + 48 | 30.8 |
| LTX-0012 | Control | 10 |
| | 1 + 48 | 11 |

### Example 6—Expansion of CD45RA+ and CD45RA− Tregs

### Materials and Methods

### Starting Material:

Leukopaks were obtained from healthy volunteers and supplied by BioIVT. PBMCs were isolated using a negative selection kit (StemCell Technologies). Briefly, unwanted fractions are labelled and targeted for removal with antibody complexes and magnetic beads and subsequently separated using a magnet.

Leukopaks were used to derive Treg populations. Leukopak-derived PBMCs were subjected to CD25 positive selection followed by a CD4 enrichment via negative selection using Human CD25 Positive Selection Cocktail and Human CD4+ T Cell Enrichment Cocktail (StemCell Technologies), respectively. Cells of interest were separated using a magnet. CD127high Depletion Cocktail (StemCell Technologies) was used to further separate the CD4+CD25+CD127−/low cell populations and cells of interest were separated using a magnet. Cell fractions were stained with flow cytometry antibodies anti-CD4 VioBlue (M-T466 Miltenyi), anti-CD25 PE (3G10, Miltenyi), anti-CD45RA FITC (T6D11, Miltenyi) and anti-CD127 APC (MB15-18C, Miltenyi) before FACS sorting. The CD4+CD25+CD127−/low Treg population was sorted into CD45RA+ and CD45RA− Treg populations. Each population (CD45RA+ and CD45RA−) from each healthy donor was split in half and then expanded under either no rapa (control) or rapa conditions (1 h+48h) as described below.

### Lentivirus

A lentiviral vector was used to transduce the cells. The vector encodes an RQR8 safety switch, a FOXP3 polypeptide and an HLA-A2-specific CAR.

### Expansion Protocol:

### “Rapa” Condition:

The Tregs were rested in Treg culture media (X-VIVO 15 media (Lonza) supplemented with 5% Human AB serum (heat-inactivated; Merck)) containing 100 nM of rapamycin for 1 hour before activation. The Tregs were then activated by adding CD3 and CD28 beads and cultured for 48 hours. At 48 hours, the cells were washed with fresh Treg culture media and centrifuged. The supernatant was removed and the cell pellet was resuspended in fresh Treg culture media. The expansion was then continued in the fresh Treg culture media (X-VIVO 15 media (Lonza) supplemented with 5% Human AB serum (heat-inactivated; Merck)) further containing IL-2 (Proleukin, Clinigen Healthcare) and lentiviral supernatant but no rapamycin. Cells were re-fed every 2 to 3 days with Treg culture media supplemented with IL-2. Cells were harvested by removing the CD3 and CD28 beads 14 days after activation.

### “Control” (No Rapa):

The Tregs were rested in Treg culture media (X-VIVO 15 media (Lonza) supplemented with 5% Human AB serum (heat-inactivated; Merck)) for 1 hour before activation. The Tregs were then activated by adding CD3 and CD28 beads and cultured for 48 hours. At 48 hours, the cells were washed with fresh Treg culture media (containing no rapamycin) and centrifuged. The supernatant was removed and the cell pellet was resuspended in fresh Treg culture media. The expansion was then continued in the fresh Treg culture media (X-VIVO 15 media (Lonza) supplemented with 5% Human AB serum (heat-inactivated; Merck)) further containing IL-2 (Proleukin, Clinigen Healthcare) and lentiviral supernatant (but no rapamycin). Cells were re-fed every 2 to 3 days with Treg culture media supplemented with IL-2. Cells were harvested by removing the CD3 and CD28 beads 14 days after activation.

### Analysis of Cell Population:

Fold expansion at day 14 was calculated by dividing the number of Tregs collected on day 14 by the number seeded on day 0.

The % of cells expressing FOXP3, Helios and CD27 in the product harvested at day 14 was determined by FACS. Tregs were first surface stained with the LIVE/DEAD™ Fixable Blue—Dead Cell Stain (Thermofisher) in PBS and then with anti-CD4 BV510 (A161A1, BioLegend), anti-CCR7 APC/Fire810 (G043H7, BioLegend), anti-CD45RA PE (H1100, BioLegend), anti-CD45RO BV750 (UCHL1, BioLegend), anti-CD27 PerCP (0323, BioLegend), anti-CD25 PE-Cy7 (B696, BioLegend), anti-RQR8 FITC (QBEND, Invitrogen) and anti-CD62L BUV737 (DREG-56, BD Biosciences) in FACS staining buffer. For intracellular staining, cells were fixed and permeabilized and stained with the anti-FoxP3 BV421 (206D, BioLegend) and anti-HELIOS APC (22F6, BioLegend). Cells were washed before acquisition on the Cytek Aurora spectral cytometer. Marker expression was analysed using FlowJo software. The following gating strategy was used: Lymphocytes>single cells>viable cells>CD4+>Marker.

### Results

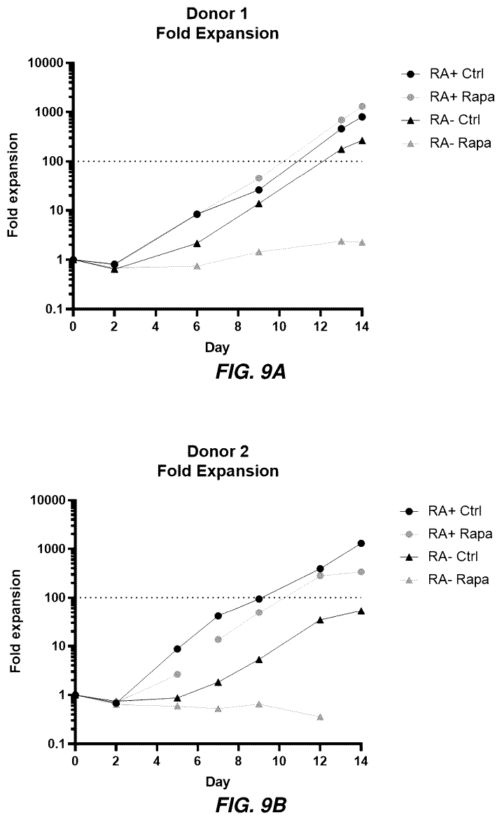

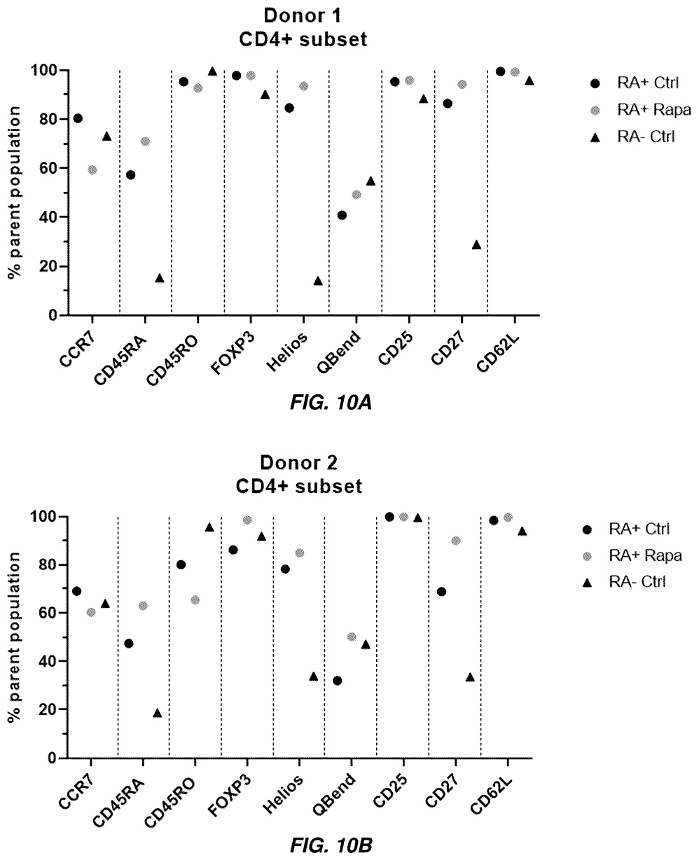

The results are shown in FIGS. 9A, 9B, 10A and 10B.

FIGS. 9A and 9B show total fold expansion of RA+ and RA− Tregs of donor 1 (FIG. 9A) and donor 2 (FIG. 9B) under the control and rapa conditions. It can be seen that the RA− Tregs do not expand under the rapa condition.

FIGS. 10A and 10B show the phenotype at day 14 for donor 1 and donor 2. There were insufficient cell numbers to assess the phenotype of RA− Tregs cultured under rapa conditions due to the lack of cell expansion. However, the RA− Tregs expanded under control conditions had a worse phenotype than the RA+ Tregs, with particularly low expression of Helios and CD27.

This is beneficial because RA+ Tregs maintain FOXP3 expression better than CD45RA− Tregs and are generally more stable than RA− Tregs and therefore in a mixed population of both RA+ and RA− Tregs, expansion under the rapa condition will selectively expand the RA+ Tregs over RA− Tregs, which results in a product with a more desirable phenotype.

### Example 7—GMP Process

### Method:

Leukopaks were obtained from healthy volunteers and liver transplant patients and supplied by BioIVT. Using a GMP-compatible process, Tregs were isolated and expanded in a culture medium suitable for Treg expansion and including IL-2 under either a “no rapa (control)” condition or a “rapa” condition. The isolated Tregs from each donor were split so that half were expanded under the no rapa condition and half expanded under the rapa condition.

In the “no rapa” (control) condition, cells were activated using anti-CD3/anti-CD28 beads on day 0 and expanded in a culture medium suitable for Treg expansion. On day 2 cells were transduced with a lentiviral vector encoding an RQR8 safety switch, FOXP3 and an HLA-A2-specific CAR. Cells were re-fed as required. Cells were harvested on day 14 and cryopreserved. There was no rapamycin present during any of the process.

In the “rapa” condition, cells were incubated for 1 hour in cell culture medium suitable for Treg expansion and containing 100 nM rapamycin. After expiry of the 1 hour pre-treatment, the cells were activated by addition of anti-CD3/anti-CD28 beads (day 0) and further incubated for 2 days in the same culture medium. On day 2, the cells were transduced with a lentiviral vector encoding an RQR8 safety switch, FOXP3 and an HLA-A2-specific CAR. At transduction, the volume in the flasks was topped up to result in the dilution of the rapamycin by 20× fold relative to the 100 nM rapamycin in the original culture medium. Cells were re-fed as required but no further rapamycin was added. Cells were harvested on day 14 and cryopreserved.

### Analysis of Cell Population:

The phenotype of the product harvested at day 14 was determined by FACS. % of CD8+ cells in the product was determined prior to cryopreservation. For the other markers, cells were thawed following cryopreservation. Tregs were first surface stained with the LIVE/DEAD™ Fixable Blue—Dead Cell Stain (Thermofisher) in PBS and then with anti-CD4 BV510 (A161A1, BioLegend), anti-CD8 BV605 (RPA-T8, Biolegend), anti-CD25 PE-Cy7 (B696, BioLegend), anti-RQR8 FITC (QBEND, Invitrogen) in FACS staining buffer. For intracellular staining, cells were fixed and permeabilized and stained with the anti-FOXP3 BV421 (206D, BioLegend) and anti-HELIOS APC (22F6, BioLegend). Cells were washed before acquisition on the Cytek Aurora spectral cytometer. Marker expression was analysed using FlowJo software. The following gating strategy was used: Lymphocytes>single cells>viable cells>CD4+>Marker.

Transduction efficiency was determined by detecting % of RQR8+ cells by FACS as described above.

Demethylation of the Treg-specific demethylated region (TSDR) of FOXP3 was determined using a methylation-specific qPCR assay.

Vector copy number (VCN) was determined using qPCR to detect copies of WPRE (present in the lentiviral vector) per transduced cell.

### Results

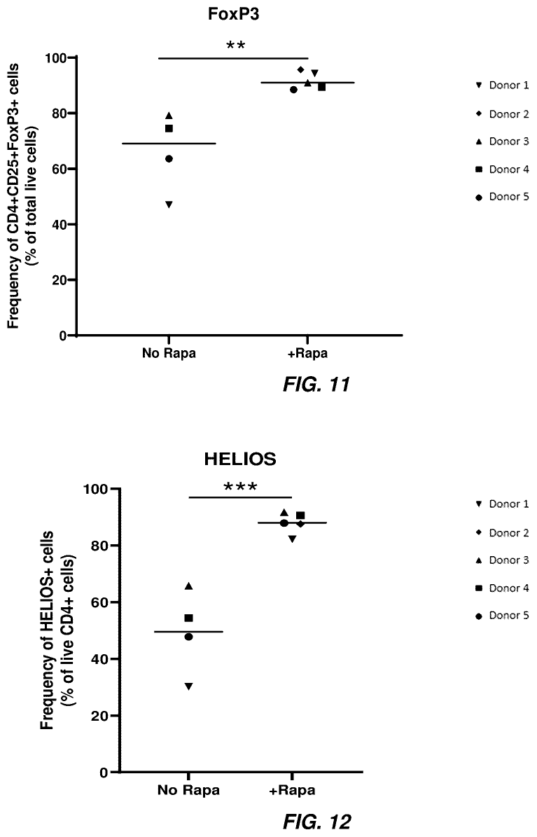

The results are shown in FIGS. 11 to 16.

FIGS. 11 and 12 show that the % of FOXP3+ and Helios+ cells respectively is greater in the product expanded under the rapa condition compared to the product expanded under the no rapa condition.

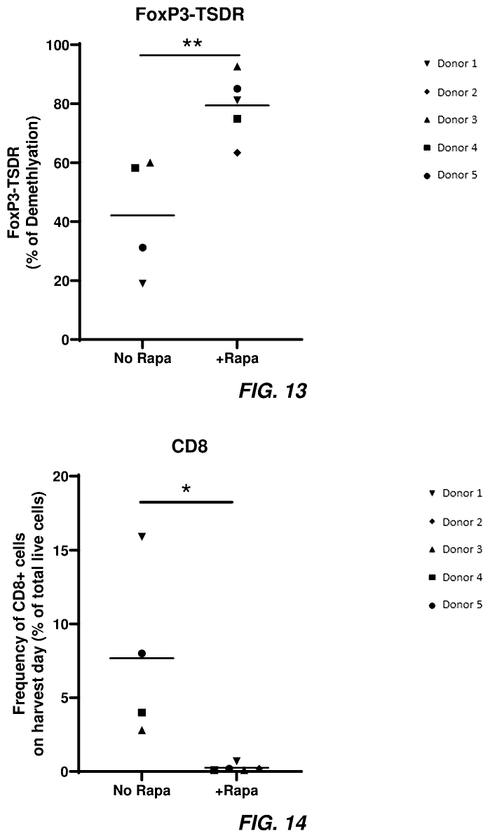

FIG. 13 shows that the % of demethylation of FOXP3-TSDR is greater in the product expanded under the rapa condition compared to the product expanded under the no rapa condition.

FIG. 14 shows that the % of CD8+ cells is lower in the product expanded under the rapa condition compared to the product expanded under the no rapa condition.

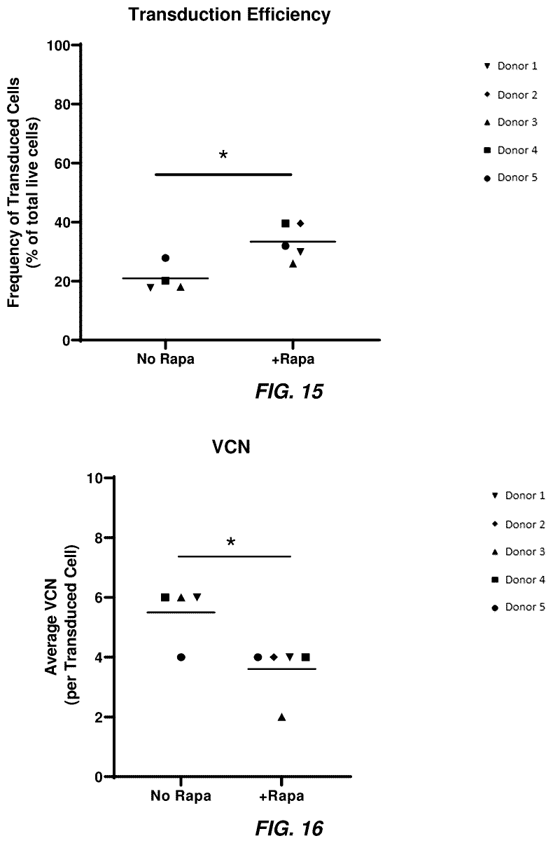

FIG. 15 shows that transduction efficiency in the product expanded under the rapa condition is greater than the product expanded under the no rapa condition.

FIG. 16 shows that the VCN in the product expanded under the rapa condition is lower than the product expanded under the no rapa condition.

1. An in vitro method for culturing regulatory T-cells (Tregs), the method comprising:
(a) contacting a population of Tregs with an mTOR inhibitor prior to activation;
(b) activating the Tregs within the population; and
(c) culturing the population of Tregs from step (b) in the presence of an mTOR inhibitor.

2. The method of claim 1, further comprising a step of isolating a population of Tregs prior to step (a), particularly a population of CD4+CD25+ Tregs.

3. The method of claim 1, wherein the mTOR inhibitor used in step (a) and/or step (c) is rapamycin or a rapalog and/or wherein the mTOR inhibitor used in step (a) is the same as the mTOR inhibitor used in step (c).

4. (canceled)

5. The method of claim 1, wherein the method does not include a step of removing the mTOR inhibitor used in step (a) prior to step (b) or step (c).

6. The method of claim 1, wherein the method does not include a step of washing the Tregs prior to step (b) or step (c).

7. The method of claim 1, wherein the mTOR inhibitor used in step (a) is present during step (b) and step (c) and particularly additional mTOR inhibitor is not added for the purpose of step (c).

8. The method of claim 1, wherein the initial concentration of the mTOR inhibitor used in step (a) and/or step (c) is from about 30 nM to about 500 nM.

9. The method of claim 1, wherein the Tregs are contacted with the mTOR inhibitor in step (a) for at least about 15 minutes, for example for about 30 minutes to about 90 minutes prior to step (b).

10. The method of claim 1, wherein the Tregs are activated by contacting them with a TCR/CD3 activator and/or a TCR co-stimulator activator.

11. The method of claim 10, wherein the TCR/CD3 activator is an anti-CD3 antibody or CD3-binding fragment thereof and/or wherein the TCR co-stimulator activator is an anti-CD28 antibody or CD28-binding fragment thereof.

12. The method of claim 1, wherein the Tregs are cultured in the presence of the mTOR inhibitor in step (c) for at least about 6 hours, for example from about 12 hours to about 72 hours, for example from about 12 hours to about 60 hours, for example from about 36 hours to about 60 hours, for example about 48 hours.

13. The method of claim 1, wherein step (c) occurs concurrently with or immediately after step (b).

14. The method of claim 1, further comprising:
(i) a step (d) of reducing the concentration of the mTOR inhibitor in contact with the population of Tregs and a step (e) of further culturing or expanding the population of Tregs in the presence of the reduced concentration of the mTOR inhibitor; or
(ii) a step (d) of removing the mTOR inhibitor from contact with the population of Tregs and a step (e) of further culturing or expanding the population of Tregs in the absence of any mTOR inhibitor;
wherein steps (d) and (e) are carried out after step (c).

15. (canceled)

16. The method of claim 14, wherein:
(i) the concentration of mTOR inhibitor is reduced to a concentration equal to or less than about 25 nM; or
(ii) the concentration of the mTOR inhibitor is reduced or the mTOR inhibitor is removed at least about 6 hours, for example from about 12 hours to about 72 hours, for example from about 12 hours to about 60 hours, for example from about 36 hours to about 60 hours after the start of step (c); or
(iii) the population of Tregs is cultured or expanded for at least about 6 days, for example from about 8 days to about 36 days, for example from about 10 days to about 14 days after the concentration of the mTOR inhibitor is reduced or the mTOR inhibitor is removed.

17. (canceled)

18. (canceled)

19. The method of claim 1, further comprising a step of introducing a heterologous nucleic acid into the Tregs, for example by transducing with a viral vector comprising the heterologous nucleic acid.

20. The method of claim 19, wherein:
(i) the heterologous nucleic acid encodes a chimeric antigen receptor (CAR) and/or a FOXP3 polypeptide and/or a safety switch and/or a polypeptide that increases persistence of the cells; or
(ii) the heterologous nucleic acid is introduced into the Tregs at least about 6 hours, for example from about 12 hours to about 72 hours, for example from about 12 hours to about 60 hours, for example from about 36 hours to about 60 hours after the start of step (b) and/or the start of step (c); or
(iii) the population of Tregs are cultured or expanded for at least about 6 days, for example from about 8 days to about 36 days, for example from about 10 days to about 14 days after the heterologous nucleic acid is introduced into the Tregs.

21. The method of claim 1, further comprising:
(i) the steps of (d) reducing the concentration of the mTOR inhibitor in contact with the population of Tregs, (e) introducing a heterologous nucleic acid into the Tregs, and (f) further culturing or expanding the population of Tregs in the presence of the reduced concentration of an mTOR inhibitor, wherein steps (d), (e) and (f) are carried out after step (c), step (e) is carried out before step (f), and step (e) is carried out before, at the same time or after step (d); or
(ii) the steps of (d) removing the mTOR inhibitor from contact with the population of Tregs, (e) introducing a heterologous nucleic acid into the Tregs, and (f) further culturing or expanding the population of Tregs in the absence of an mTOR inhibitor, wherein steps (d), (e) and (f) are carried out after step (c), step (e) is carried out before step (f), and step (e) is carried out after step (d).

22. (canceled)

23. The method of claim 21, wherein option (i) is chosen and the concentration of the mTOR inhibitor is reduced to a concentration equal to or less than about 25 nM simultaneously with introducing the heterologous nucleic acid into the Tregs within the population.

24. (canceled)

25. (canceled)

26. The method of claim 1, wherein the population of Tregs are harvested and optionally cryopreserved from about 8 days to about 36 days after the start of step (b), for example from about 8 days to about 22 days after the start of step (b).

27. A product obtained by and/or obtainable by the method of claim 1.
